(19) United States
(12) Patent Application Publication
(10) Pub. No.: US 2016/0263117 A1
(43) Pub. Date: Sep. 15, 2016

(54) COMPOSITIONS AND METHODS FOR CARDIOVASCULAR DISEASE

(71) Applicant: The General Hospital Corporation, Boston, MA (US)

(72) Inventors: Paul B. Yu, Boston, MA (US); Matthias Derwall, Moers (DE); Kenneth D. Bloch, Chestnut Hill, MA (US); Rajeev Malhotra, Boston, MA (US)

(21) Appl. No.: 15/070,774

(22) Filed: Mar. 15, 2016

Related U.S. Application Data
(63) Continuation of application No. 13/979,943, filed on Oct. 22, 2013, now abandoned.
(60) Provisional application No. 61/434,932, filed on Jan. 21, 2011.

(51) Int. Cl. A61K 31/519; A61K 31/4709; A61K 31/496; A61K 31/5377

(52) U.S. Cl. CPC A61K 31/519; A61K 31/5377; A61K 31/4709; A61K 31/496

(57) ABSTRACT

The present invention provides small molecule inhibitors of BMP signaling. These compounds may be used to reduce circulating levels of ApoB-100 or LDL. These compounds may also be used to treat or prevent acquired or congenital hypercholesterolemia or hyperlipoproteinemia; diseases, disorders, or syndromes associated with defects in lipid absorption or metabolism; or diseases, disorders, or syndromes caused by hyperlipidemia.

### RELATED APPLICATIONS

This application claims the benefit of priority to U.S. Provisional Patent Application No. 61/434,932, filed Jan. 21, 2011, which application is hereby incorporated by reference in its entirety

### STATEMENT REGARDING FEDERALLY SPONSORED RESEARCH OR DEVELOPMENT

This invention was supported in part by the United States Government under National Institutes of Health Grant NIH/NHLBI 5K08HL079943. The Government may have certain rights in this invention.

### BACKGROUND OF THE INVENTION

Signaling involving the Transforming Growth Factor β (TGF-β) superfamily of ligands is central to a wide range of cellular processes, including cell growth, differentiation, and apoptosis. TGF-β signaling involves binding of a TGF-β ligand to a type II receptor (a serine/threonine kinase), which recruits and phosphorylates a type I receptor. The type I receptor then phosphorylates a receptor-regulated SMAD (R-SMAD; e.g., SMAD1, SMAD2, SMAD3, SMAD5, SMAD8 or SMAD9), which binds to SMAD4, and the SMAD complex then enters the nucleus where it plays a role in transcriptional regulation. The TGF superfamily of ligands includes two major branches, characterized by TGF-β/activin/nodal and Bone Morphogenetic Proteins (BMPs).

Signals mediated by bone morphogenetic protein (BMP) ligands serve diverse roles throughout the life of vertebrates. During embryogenesis, the dorsoventral axis is established by BMP signaling gradients formed by the coordinated expression of ligands, receptors, co-receptors, and soluble antagonists (Massague et al. Nat. Rev. Mol. Cell. Biol. 1:169-178, 2000). Excess BMP signaling causes ventralization, an expansion of ventral at the expense of dorsal structures, while diminished BMP signaling causes dorsalization, an expansion of dorsal at the expense of ventral structures (Nguyen et al. Dev. Biol. 199: 93-110, 1998; Furthauer et al. Dev. Biol. 214:181-196, 1999; Mintzer et al. Development 128:859-869, 2001; Schmid et al. Development 127:957-967, 2000). BMPs are key regulators of gastrulation, mesoderm induction, organogenesis, and endochondral bone formation, and regulate the fates of multipotent cell populations (Zhao, Genesis 35:43-56, 2003). BMP signals also play critical roles in physiology and disease, and are implicated in primary pulmonary hypertension, hereditary hemorrhagic telangiectasia syndrome, fibrodysplasia ossificans progressiva, and juvenile polyposis syndrome (Waite et al. Nat. Rev. Genet. 4:763-773, 2003; Papanikolaou et al. Nat. Genet. 36:77-82, 2004; Shore et al. Nat. Genet. 38:525-527, 2006).

The BMP signaling family is a diverse subset of the TGF-β superfamily (Sebald et al. Biol. Chem. 385:697-710, 2004). Over twenty known BMP ligands are recognized by three distinct type II (BMPRII, ActRIIa, and ActRIIb) and at least four type I (ALK1, ALK2, ALK3, and ALK6) receptors. Dimeric ligands facilitate assembly of receptor heteromers, allowing the constitutively-active type II receptor serine/threonine kinases to phosphorylate type I receptor serine/threonine kinases. Activated type I receptors phosphorylate BMP-responsive (BR-) SMAD effectors (SMADs 1, 5, and 8) to facilitate nuclear translocation in complex with SMAD4, a co-SMAD that also facilitates TGF signaling. In addition, BMP signals can activate intracellular effectors such as MAPK p38 in a SMAD-independent manner (Nohe et al. Cell Signal 16:291-299, 2004). Soluble BMP antagonists, such as noggin, chordin, gremlin, and follistatin, limit BMP signaling by ligand sequestration.

A role for BMP signals in regulating expression of hepcidin, a peptide hormone and central regulator of systemic iron balance, has also been suggested (Pigeon et al. J. Biol. Chem. 276:7811-7819, 2001; Fraenkel et al. J. Clin. Invest. 115:1532-1541, 2005; Nicolas et al. Proc. Natl. Acad. Sci. U.S.A. 99:4596-4601, 2002; Nicolas et al. Nat. Genet. 34:97-101, 2003). Hepcidin binds and promotes degradation of ferroportin, the sole iron exporter in vertebrates. Loss of ferroportin activity prevents mobilization of iron to the bloodstream from intracellular stores in enterocytes, macrophages, and hepatocytes (Nemeth et al. Science 306:2090-2093, 2004). The link between BMP signaling and iron metabolism represents a potential target for therapeutics.

Given the tremendous structural diversity of the BMP and TGF-β superfamily at the level of ligands (>25 distinct ligands at present) and receptors (four type I and three type II receptors that recognize BMPs), and the heterotetrameric manner of receptor binding, traditional approaches for inhibiting BMP signals via soluble receptors, endogenous inhibitors, or neutralizing antibodies are not practical or effective. Endogenous inhibitors such as noggin and follistatin have limited specificity for ligand subclasses. Single receptors have limited affinity for ligand, whereas ligand heterotetramers exhibit rather precise specificity for particular ligands. Neutralizing antibodies are specific for particular ligands or receptors and are also limited by the structural diversity of this signaling system. Thus, there is a need in the art for pharmacologic agents that specifically antagonize BMP signaling pathways and that can be used to manipulate these pathways in therapeutic or experimental applications, such as those listed above.

### SUMMARY OF THE INVENTION

In one aspect, the invention provides compounds that inhibit BMP-induced phosphorylation of SMAD1/5/8 including compounds represented by general formula I:

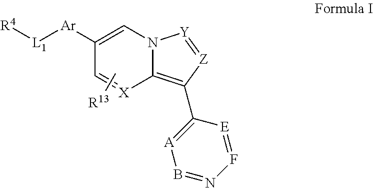

wherein
X is selected from CR¹⁵ and N;
Y is selected from CR¹⁵ and N;
Z is selected from CR³ and N;
Ar is selected from substituted or unsubstituted aryl and heteroaryl, e.g., a six-membered ring, such as phenyl;
L₁ is absent or selected from substituted or unsubstituted alkyl and heteroalkyl;
A and B, independently for each occurrence, are selected from CR¹⁶ and N, preferably CR¹⁶, e.g., CH;
E and F, independently for each occurrence, are selected from CR⁵ and N, preferably CR⁵;
preferably chosen such that no more than two of A, B, E, and F are N;
R³ represents a substituent, e.g., selected from H and substituted or unsubstituted alkyl, heteroalkyl, cycloalkyl, halogen, hydroxyl, alkoxyl, alkylthio, acyloxy, acylamino, carbamate, cyano, sulfonyl, sulfoxido, sulfamoyl, or sulfonamido, e.g., lower alkyl;
R⁴ is selected from substituted or unsubstituted alkyl, alkenyl, alkynyl, heteroalkyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido, e.g., substituted or unsubstituted alkenyl, alkynyl, cycloalkyl, heterocyclyl, aryl, heteroaryl, acyl, carboxyl, ester, acyloxy, amino, acylamino, carbamate, amido, amidino, sulfonyl, sulfoxido, sulfamoyl, or sulfonamido, preferably substituted or unsubstituted heterocyclyl or heteroaryl;
R⁵, independently for each occurrence, represents a substituent, e.g., selected from H and substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano), or two occurrences of R⁵ taken together with the atoms to which they are attached form a substituted or unsubstituted 5- or 6-membered cycloalkyl, heterocycloalkyl, aryl, or heteroaryl ring, preferably an aryl or heteroaryl ring, e.g., a substituted or unsubstituted benzo ring;
R¹³ is absent or represents 1-2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably substituted or unsubstituted alkyl, heteroalkyl, halogen, hydroxyl, alkoxyl, alkylthio, acyloxy, acylamino, carbamate, or cyano;
R¹⁵, independently for each occurrence, represents a substituent, e.g., selected from H and substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably H or substituted or unsubstituted alkyl, heteroalkyl, halogen, hydroxyl, alkoxyl, alkylthio, acyloxy, acylamino, carbamate, or cyano;
R¹⁶, independently for each occurrence, represents a substituent, e.g., selected from H and substituted or unsubstituted alkyl, alkenyl, alkynyl, heteroalkyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido, preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, or cyano,
   or a pharmaceutically acceptable salt, ester, or prodrug thereof.

In certain embodiments, either Y is N or Ar comprises a nitrogen atom in the ring.

In certain embodiments, E and F are each CR⁵, and both instances of R⁵ together with the intervening atoms form a 5-, 6-, or 7-membered ring optionally substituted by substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano). In certain embodiments, E and F together form a substituted or unsubstituted 6-membered cycloalkyl, heterocyclyl, aryl or heteroaryl ring (e.g., a pyridine, piperidine, pyran, or piperazine ring, etc.). In certain such embodiments, the ring comprises one to four amine groups, while in other embodiments, the ring is a substituted or unsubstituted benzo ring

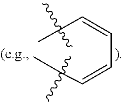 [SMILES: */C=C\C=C/*]

In certain such embodiments, the ring is substituted, e.g., by optionally substituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably alkyl, alkenyl, heteroalkyl, halogen, acyl, carboxyl, ester, hydroxyl, alkoxyl, alkylthio, acyloxy, amino, acylamino, carbamate, amido, amidino, or cyano).

In certain embodiments, Ar represents substituted or unsubstituted heteroaryl e.g., pyrrole, furan, thiophene, imidazole, oxazole, thiazole, pyrazole, pyridine, pyrazine, pyridazine, quinoline, and pyrimidine, In certain embodiments, Ar represents substituted or unsubstituted aryl, such as phenyl. In certain embodiments, Ar is a 6-membered ring, such as a phenyl ring, e.g., in which L₁ is disposed on the para-position of Ar relative to the bicyclic core.

In certain embodiments as discussed above, substituents on Ar are selected from substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano).

In certain embodiments, L₁ represents a linker Mₖ, wherein k is an integer from 1-8, preferably from 2-4, and each M represents a unit selected from C(R¹⁸)₂, NR¹⁹, S, SO₂, or O, preferably selected so that no two heteroatoms occur in adjacent positions, more preferably with at least two carbon atoms between any nitrogen atom and another heteroatom; wherein R¹⁸, independently for each occurrence, is selected from H and substituted or unsubstituted alkyl, heteroalkyl, cycloalkyl, heterocyclyl, cycloalkylalkyl, heterocyclylalkyl, hydroxyl, alkoxyl, alkylthio, acyloxy, amino, acylamino, carbamate, amido, amidino, cyano, sulfonyl, sulfoxido, sulfamoyl, or sulfonamido, preferably H or lower alkyl; and R¹⁹ is selected from H and substituted or unsubstituted alkyl, cycloalkyl, heterocyclyl, heterocyclylalkyl, oxide, amino, acylamino, carbamate, amido, amidino, sulfonyl, sulfamoyl, or sulfonamido, preferably H or lower alkyl.

In certain embodiments, L₁ is absent. In certain embodiments, L₁ is selected from substituted or unsubstituted alkyl (e.g., C₁-C₈ chains, preferably C₂-C₄ chains) and heteroalkyl. In certain such embodiments, L₁ has a structure

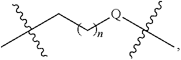

wherein n is an integer from 0 to 4, and Q is selected from CR¹⁰R¹¹, NR¹², O, S, S(O), and SO₂; R¹⁰ and R¹¹, independently for each occurrence, are selected from H and substituted or unsubstituted alkyl, heteroalkyl, cycloalkyl, heterocyclyl, cycloalkylalkyl, heterocyclylalkyl, hydroxyl, alkoxyl, alkylthio, acyloxy, amino, acylamino, carbamate, amido, amidino, cyano, sulfonyl, sulfoxido, sulfamoyl, or sulfonamido, preferably H or lower alkyl; and R¹² is selected from H and substituted or unsubstituted alkyl, cycloalkyl, heterocyclyl, heterocyclylalkyl, oxide, amino, acylamino, carbamate, amido, amidino, sulfonyl, sulfamoyl, or sulfonamido, preferably H or lower alkyl. In certain embodiments, L₁ has a structure

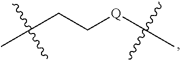

wherein Q is CH₂, NH, S, SO₂, or O, preferably O.

In certain embodiments, R⁴ is

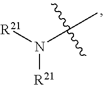

wherein R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamido, preferably H or lower alkyl.

In certain embodiments, R⁴ is heterocyclyl, e.g., comprising one or two heteroatoms, such as N, S or O (e.g., piperidine, piperazine, pyrrolidine, morpholine, lactone, or lactam). In certain such embodiments, R⁴ is heterocyclyl comprising one nitrogen atom, e.g., piperidine or pyrrolidine, such as

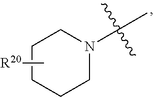

wherein R²⁰ is absent or represents from 1-4 substituents on the ring to which it is attached, e.g., selected from substituted or unsubstituted alkyl, heteroaryl, aralkyl, cycloalkyl, heterocyclyl, aryl, heteroaryl, heteroaralkyl, cycloalkylalkyl, heterocyclylalkyl, acyl, hydroxyl, alkoxyl, alkylthio, acyloxy, sulfonyl, sulfoxido, sulfamoyl, and sulfonamido, preferably H or lower alkyl. In certain embodiments, R⁴ is heterocyclyl comprising two nitrogen atoms, e.g., piperazine. In certain embodiments, R⁴ is heterocyclyl comprising a nitrogen and an oxygen atom, e.g., morpholine.

In certain embodiments, R⁴ is a heterocyclyl or heteroaryl that includes an amine within the atoms of the ring, e.g., pyridyl, imidazolyl, pyrrolyl, piperidyl, pyrrolidyl, piperazyl, oxazolyl, isoxazolyl, thiazolyl, etc., and/or bears an amino substituent. In certain embodiments, R⁴ is

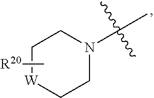

wherein R²⁰ is as defined above; W represents a bond or is selected from C(R²¹)₂, O, or NR²¹; and R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamido, preferably H or lower alkyl.

In certain preferred embodiments, L₁ is absent and Ar—R⁴ has a structure

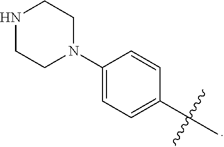 [SMILES: *c1ccc(N2CCNCC2)cc1]

In certain embodiments as discussed above, substituents on R⁴ are selected from substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano).

In certain embodiments, L₁ is absent and R⁴ is directly attached to Ar. In embodiments wherein R⁴ is a six-membered ring directly attached to Ar and bears an amino substituent at the 4-position of the ring relative to N.

In certain embodiments, L₁-R⁴ comprises a basic nitrogen-containing group, e.g., either L₁ comprises nitrogen-containing heteroalkyl or an amine-substituted alkyl, or R⁴ comprises a substituted or unsubstituted nitrogen-containing heterocyclyl or heteroaryl and/or is substituted with an amine substituent. In certain such embodiments, the pKₐ of the conjugate acid of the basic nitrogen-containing group is 6 or higher, or even 8 or higher.

In certain embodiments, L₁ has a structure

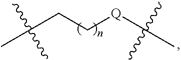

wherein n is an integer from 0 to 4, and R⁴ is heterocyclyl. In certain such embodiments, E and F together form a ring, e.g., a benzo ring, while in other embodiments, E and F do not form a ring.

In certain embodiments, L₁ is absent and R⁴ is heterocyclyl, especially a nitrogen-containing heterocyclyl. In certain such embodiments, E and F together form a ring, e.g., a benzo ring, while in other embodiments, E and F do not form a ring. In certain embodiments, L₁ is absent and R⁴ is piperidine, piperazine, pyrrolidine, or morpholine.

In certain of the embodiments disclosed above, if L₁ is alkyl or heteroalkyl and R⁴ is heterocyclyl, especially a nitrogen-containing heterocyclyl, then E and F together form a ring, e.g., a benzo ring. In certain of the embodiments disclosed above, if L₁ has a structure

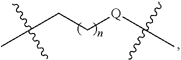

wherein n is an integer from 0 to 4 (especially from 1-2) and Q is S or O, then E and F together form a ring, e.g., a benzo ring.

In certain embodiments, either E and F are both CR⁵ and both occurrences of R⁵ taken together with E and F form a ring, e.g., a benzo ring, or L₁ is absent. In certain such embodiments, R⁴ is selected from substituted or unsubstituted alkenyl, alkynyl, cycloalkyl, heterocyclyl, aryl, heteroaryl, acyl, carboxyl, ester, acyloxy, amino, acylamino, carbamate, amido, amidino, sulfonyl, sulfoxido, sulfamoyl, and sulfonamido. In certain embodiments, either E and F are both CR⁵ and both occurrences of R⁵ taken together with E and F form a ring, e.g., a benzo ring, or R⁴ is selected from substituted or unsubstituted cycloalkyl, aryl, heteroaryl, acyl, carboxyl, ester, acyloxy, amino, acylamino, carbamate, amido, amidino, sulfonyl, sulfoxido, sulfamoyl, and sulfonamido.

In certain of the embodiments disclosed above, if L₁ is absent, R⁴ is cycloalkyl or heterocyclyl (e.g., a nitrogen-containing heterocycle, such as piperidine, piperazine, pyrrolidine, morpholine, etc.).

In certain of the embodiments disclosed above, if L₁ is heteroalkyl and R⁴ is heterocyclyl (especially a nitrogen-containing heterocycle), then Y is CR¹⁵, wherein R¹⁵ is as defined above. In certain of the embodiments disclosed above, if L₁ is heteroalkyl and R⁴ is piperidine, then Y is CR¹⁵, wherein R¹⁵ is as defined above. In certain embodiments wherein Y is CR¹⁵, R¹⁵ is selected from H, lower alkyl, heteroalkyl, and ester (e.g., lower alkyl ester, such as methyl ester).

In certain of the embodiments disclosed above, if L₁ is heteroalkyl and R⁴ is heterocyclyl (especially nitrogen-containing heterocyclyl), then X is CR¹⁵, wherein R¹⁵ is as defined above. In certain of the embodiments disclosed above, if L₁ is heteroalkyl and R⁴ is piperidine, then X is CR¹⁵, wherein R¹⁵ is as defined above. In certain embodiments wherein X is R¹⁵, R¹⁵ is selected from H, lower alkyl, and heteroalkyl.

In certain of the embodiments disclosed above, if L₁ is heteroalkyl and R⁴ is heterocyclyl (especially nitrogen-containing heterocyclyl), then Z is CR³, wherein R³ is as defined above. In certain of the embodiments disclosed above, if L₁ is heteroalkyl and R⁴ is piperidine, then Z is CR³, wherein R³ is as defined above. In certain embodiments wherein Z is CR³, R³ is selected from H, lower alkyl, and heteroalkyl.

In certain of the embodiments disclosed above, if L₁ is heteroalkyl and R⁴ is heterocyclyl (especially a nitrogen-containing heterocycle, such as piperidine), R¹³ represents 2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido.

In certain of the embodiments disclosed above, if L₁ is heteroalkyl and R⁴ is heterocyclyl (especially a nitrogen-containing heterocycle, such as piperidine), Ar represents substituted or unsubstituted heteroaryl (e.g., pyrrole, furan, thiophene, imidazole, oxazole, thiazole, pyrazole, pyridine, pyrazine, pyridazine, quinoline, and pyrimidine). In certain such embodiments, Ar is substituted with one or more substituents selected from alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido.

In certain of the embodiments disclosed above, if L₁ is heteroalkyl and R⁴ is heterocyclyl (e.g., piperidine, piperazine, pyrrolidine, morpholine, lactones, lactams, and the like), R⁴ is substituted with one or more substituents selected from alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido.

In certain of the embodiments disclosed above, compounds have one or more of the following features:

either Y is N or Ar comprises a nitrogen atom in the ring;

L₁ is absent;

E and F together form a ring;

R⁴ is cycloalkyl, aryl, or heteroaryl;

X is CR¹⁵;

Y is CR¹⁵;

Z is CR³;

R¹³ represents 1-2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido;

Ar represents substituted or unsubstituted heteroaryl (e.g., pyrrole, furan, thiophene, imidazole, oxazole, thiazole, pyrazole, pyridine, pyrazine, pyridazine, quinoline, and pyrimidine);

Ar is substituted with one or more substituents selected from alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido; and

R⁴ is substituted with one or more substituents selected from alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido.

In one aspect, the invention provides compounds that inhibit BMP-induced phosphorylation of SMAD1/5/8 including compounds represented by general formula II:

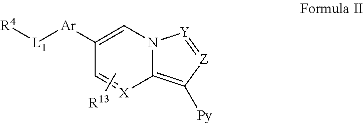

wherein
X is selected from CR¹⁵ and N;
Y is selected from CR¹⁵ and N;
Z is selected from CR³ and N;
Ar is selected from substituted or unsubstituted aryl and heteroaryl, e.g., a six-membered ring, such as phenyl;
L₁ is absent or selected from substituted or unsubstituted alkyl and heteroalkyl;
Py is substituted or unsubstituted 4-pyridinyl or 4-quinolinyl, e.g., optionally substituted with substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, amino, acylamino, carbamate, amido, amidino, cyano, sulfonyl, sulfoxido, sulfamoyl, or sulfonamido; and
R³ represents a substituent, e.g., selected from H and substituted or unsubstituted alkyl, heteroalkyl, cycloalkyl, halogen, hydroxyl, alkoxyl, alkylthio, acyloxy, acylamino, carbamate, cyano, sulfonyl, sulfoxido, sulfamoyl, or sulfonamido, e.g., lower alkyl;
R⁴ is selected from substituted or unsubstituted alkyl, alkenyl, alkynyl, heteroalkyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido, e.g., substituted or unsubstituted alkenyl, alkynyl, cycloalkyl, heterocyclyl, aryl, heteroaryl, acyl, carboxyl, ester, acyloxy, amino, acylamino, carbamate, amido, amidino, sulfonyl, sulfoxido, sulfamoyl, or sulfonamido, preferably substituted or unsubstituted heterocyclyl or heteroaryl;
R⁵, independently for each occurrence, represents a substituent, e.g., selected from H and substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano), or two occurrences of R⁵ taken together with the atoms to which they are attached form a substituted or unsubstituted 5- or 6-membered cycloalkyl, heterocycloalkyl, aryl, or heteroaryl ring, preferably an aryl or heteroaryl ring, e.g., a substituted or unsubstituted benzo ring;
R¹³ is absent or represents 1-2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably substituted or unsubstituted alkyl, heteroalkyl, halogen, hydroxyl, alkoxyl, alkylthio, acyloxy, acylamino, carbamate, or cyano;
R¹⁵, independently for each occurrence, represents a substituent, e.g., selected from H and substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido, preferably H or substituted or unsubstituted alkyl, heteroalkyl, halogen, hydroxyl, alkoxyl, alkylthio, acyloxy, acylamino, carbamate, or cyano;
R¹⁶, independently for each occurrence, represents a substituent, e.g., selected from H and substituted or unsubstituted alkyl, alkenyl, alkynyl, heteroalkyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido, preferably H or substituted or unsubstituted alkyl, alkenyl, heteroalkyl, halogen, acyl, carboxyl, ester, hydroxyl, alkoxyl, alkylthio, acyloxy, amino, acylamino, carbamate, amido, or cyano,
   or a pharmaceutically acceptable salt, ester, or prodrug thereof.

In certain embodiments, either Y is N or Ar comprises a nitrogen atom in the ring.

In certain embodiments, Py is substituted by substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano).

In certain embodiments, Ar represents substituted or unsubstituted heteroaryl e.g., pyrrole, furan, thiophene, imidazole, oxazole, thiazole, pyrazole, pyridine, pyrazine, pyridazine, quinoline, and pyrimidine. In certain embodiments, Ar represents substituted or unsubstituted aryl, such as phenyl. In certain embodiments, Ar is a 6-membered ring, such as a phenyl ring, e.g., in which L₁ is disposed on the para-position of Ar relative to the bicyclic core.

In certain embodiments as discussed above, substituents on Ar are selected from substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano).

In certain embodiments, L₁ represents a linker Mₖ, wherein k is an integer from 1-8, preferably from 2-4, and each M represents a unit selected from C(R¹⁸)₂, NR¹⁹, S, SO₂, or O, preferably selected so that no two heteroatoms occur in adjacent positions, more preferably with at least two carbon atoms between any nitrogen atom and another heteroatom; wherein R¹⁸, independently for each occurrence, is selected from H and substituted or unsubstituted alkyl, heteroalkyl, cycloalkyl, heterocyclyl, cycloalkylalkyl, heterocyclylalkyl, hydroxyl, alkoxyl, alkylthio, acyloxy, amino, acylamino, carbamate, amido, amidino, cyano, sulfonyl, sulfoxido, sulfamoyl, or sulfonamido, preferably H or lower alkyl; and R¹⁹ is selected from H and substituted or unsubstituted alkyl, cycloalkyl, heterocyclyl, heterocyclylalkyl, oxide, amino, acylamino, carbamate, amido, amidino, sulfonyl, sulfamoyl, or sulfonamido, preferably H or lower alkyl.

In certain embodiments, L₁ is absent. In certain embodiments, L₁ is selected from substituted or unsubstituted alkyl (e.g., C₁-C₈ chains, preferably C₂-C₄ chains) and heteroalkyl. In certain such embodiments, L₁ has a structure

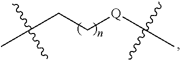

wherein n is an integer from 0 to 4, and Q is selected from CR¹⁰R¹¹, NR¹², O, S, S(O), and SO₂; R¹⁰ and R¹¹, independently for each occurrence, are selected from H and substituted or unsubstituted alkyl, heteroalkyl, cycloalkyl, heterocyclyl, cycloalkylalkyl, heterocyclylalkyl, hydroxyl, alkoxyl, alkylthio, acyloxy, amino, acylamino, carbamate, amido, amidino, cyano, sulfonyl, sulfoxido, sulfamoyl, or sulfonamido, preferably H or lower alkyl; and R¹² is selected from H and substituted or unsubstituted alkyl, cycloalkyl, heterocyclyl, heterocyclylalkyl, oxide, amino, acylamino, carbamate, amido, amidino, sulfonyl, sulfamoyl, or sulfonamido, preferably H or lower alkyl. In certain embodiments, L₁ has a structure

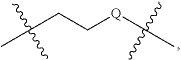

wherein Q is CH₂, NH, S, SO₂, or O, preferably O.

In certain embodiments, R⁴ is

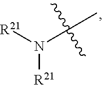

wherein R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamido, preferably H or lower alkyl.

In certain embodiments, R⁴ is heterocyclyl, e.g., comprising one or two heteroatoms, such as N, S or O (e.g., piperidine, piperazine, pyrrolidine, morpholine, lactone, or lactam). In certain such embodiments, R⁴ is heterocyclyl comprising one nitrogen atom, e.g., piperidine or pyrrolidine, such as

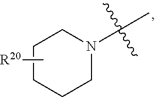

wherein R²⁰ is absent or represents from 1-4 substituents on the ring to which it is attached, e.g., selected from substituted or unsubstituted alkyl, heteroaryl, aralkyl, cycloalkyl, heterocyclyl, aryl, heteroaryl, heteroaralkyl, cycloalkylalkyl, heterocyclylalkyl, acyl, hydroxyl, alkoxyl, alkylthio, acyloxy, sulfonyl, sulfoxido, sulfamoyl, and sulfonamido, preferably H or lower alkyl. In certain embodiments, R⁴ is heterocyclyl comprising two nitrogen atoms, e.g., piperazine. In certain embodiments, R⁴ is heterocyclyl comprising a nitrogen and an oxygen atom, e.g., morpholine.

In certain embodiments, R⁴ is a heterocyclyl or heteroaryl that includes an amine within the atoms of the ring, e.g., pyridyl, imidazolyl, pyrrolyl, piperidyl, pyrrolidyl, piperazyl, oxazolyl, isoxazolyl, thiazolyl, etc., and/or bears an amino substituent. In certain embodiments, R⁴ is

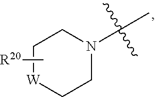

wherein R²⁰ is as defined above; W represents a bond or is selected from C(R²¹)₂, O, or NR²¹; and R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamido, preferably H or lower alkyl.

In certain embodiments as discussed above, substituents on R⁴ are selected from substituted or unsubstituted alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido (preferably substituted or unsubstituted alkyl, alkenyl, heteroalkyl, halogen, acyl, carboxyl, ester, hydroxyl, alkoxyl, alkylthio, acyloxy, amino, acylamino, carbamate, amido, amidino, or cyano).

In certain embodiments, L₁ is absent and R⁴ is directly attached to Ar. In embodiments wherein R⁴ is a six-membered ring directly attached to Ar and bears an amino substituent at the 4-position of the ring relative to N, the N and amine substituents may be disposed trans on the ring.

In certain embodiments, L₁-R⁴ comprises a basic nitrogen-containing group, e.g., either L₁ comprises nitrogen-containing heteroalkyl or an amine-substituted alkyl, or R⁴ comprises a substituted or unsubstituted nitrogen-containing heterocyclyl or heteroaryl and/or is substituted with an amine substituent. In certain such embodiments, the pKₐ of the conjugate acid of the basic nitrogen-containing group is 6 or higher, or even 8 or higher.

In certain embodiments, L₁ has a structure

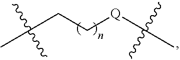

wherein n is an integer from 0 to 4, and R⁴ is heterocyclyl. In certain such embodiments, Py is 4-quinolinyl, while in other embodiments, Py is 4-pyridinyl.

In certain embodiments, L₁ is absent and R⁴ is heterocyclyl, especially a nitrogen-containing heterocyclyl. In certain such embodiments, Py is 4-quinolinyl, while in other embodiments, Py is 4-pyridinyl. In certain embodiments, L₁ is absent and R⁴ is piperidine, piperazine, pyrrolidine, or morpholine.

In certain of the embodiments disclosed above, if L₁ is alkyl or heteroalkyl and R⁴ is heterocyclyl, especially a nitrogen-containing heterocyclyl, then Py is 4-quinolinyl. In certain of the embodiments disclosed above, if L₁ has a structure

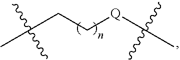

wherein n is an integer from 0 to 4 (especially from 1-2) and Q is S or O, then Py is 4-quinolinyl.

In certain embodiments, either Py is 4-quinolinyl, or L₁ is absent. In certain such embodiments, R⁴ is selected from substituted or unsubstituted alkenyl, alkynyl, cycloalkyl, heterocyclyl, aryl, heteroaryl, acyl, carboxyl, ester, acyloxy, amino, acylamino, carbamate, amido, amidino, sulfonyl, sulfoxido, sulfamoyl, and sulfonamido. In certain embodiments, either Py is 4-quinolinyl, or R⁴ is selected from substituted or unsubstituted cycloalkyl, aryl, heteroaryl, acyl, carboxyl, ester, acyloxy, amino, acylamino, carbamate, amido, amidino, sulfonyl, sulfoxido, sulfamoyl, and sulfonamido.

In certain of the embodiments disclosed above, if L₁ is absent, R⁴ is cycloalkyl or heterocyclyl (e.g., a nitrogen-containing heterocycle, such as piperidine, piperazine, pyrrolidine, morpholine, etc.).

In certain of the embodiments disclosed above, if L₁ is heteroalkyl and R⁴ is heterocyclyl (especially a nitrogen-containing heterocycle), then Y is CR¹⁵, wherein R¹⁵ is as defined above. In certain of the embodiments disclosed above, if L₁ is heteroalkyl and R⁴ is piperidine, then Y is CR¹⁵, wherein R¹⁵ is as defined above. In certain embodiments wherein Y is CR¹⁵, R¹⁵ is selected from H, lower alkyl, heteroalkyl, and ester (e.g., lower alkyl ester, such as methyl ester).

In certain of the embodiments disclosed above, if L₁ is heteroalkyl and R⁴ is heterocyclyl (especially nitrogen-containing heterocyclyl), then X is CR¹⁵, wherein R¹⁵ is as defined above. In certain of the embodiments disclosed above, if L₁ is heteroalkyl and R⁴ is piperidine, then X is CR¹⁵, wherein R¹⁵ is as defined above. In certain embodiments wherein X is R¹⁵, R¹⁵ is selected from H, lower alkyl, and heteroalkyl.

In certain of the embodiments disclosed above, if L₁ is heteroalkyl and R⁴ is heterocyclyl (especially nitrogen-containing heterocyclyl), Z is CR³, wherein R³ is as defined above. In certain of the embodiments disclosed above, if L₁ is heteroalkyl and R⁴ is piperidine, then Z is CR³, wherein R³ is as defined above. In certain embodiments wherein Z is CR³, R³ is selected from H, lower alkyl, and heteroalkyl.

In certain of the embodiments disclosed above, if L₁ is heteroalkyl and R⁴ is heterocyclyl (especially a nitrogen-containing heterocycle, such as piperidine), R¹³ represents 2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido.

In certain of the embodiments disclosed above, if L₁ is heteroalkyl and R⁴ is heterocyclyl (especially a nitrogen-containing heterocycle, such as piperidine), Ar represents substituted or unsubstituted heteroaryl (e.g., pyrrole, furan, thiophene, imidazole, oxazole, thiazole, pyrazole, pyridine, pyrazine, pyridazine, quinoline, and pyrimidine). In certain such embodiments, Ar is substituted with one or more substituents selected from alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido.

In certain of the embodiments disclosed above, if L₁ is heteroalkyl and R⁴ is heterocyclyl (e.g., piperidine, piperazine, pyrrolidine, morpholine, lactones, lactams, and the like), R⁴ is substituted with one or more substituents selected from alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido.

In certain of the embodiments disclosed above, compounds have one or more of the following features:

either Y is N or Ar comprises a nitrogen atom in the ring;

L₁ is absent;

Py is 4-quinolinyl;

R⁴ is cycloalkyl, aryl, or heteroaryl;

X is CR¹⁵;

Y is CR¹⁵;

Z is CR³;

R¹³ represents 1-2 substituents on the ring to which it is attached and, independently for each occurrence, is selected from substituted or unsubstituted alkyl, heteroalkyl, cycloalkyl, heterocyclyl, cycloalkylalkyl, heterocyclylalkyl, halogen, hydroxyl, alkoxyl, alkylthio, acyloxy, acylamino, carbamate, cyano, sulfonyl, sulfoxido, sulfamoyl, or sulfonamido;

Ar represents substituted or unsubstituted heteroaryl (e.g., pyrrole, furan, thiophene, imidazole, oxazole, thiazole, pyrazole, pyridine, pyrazine, pyridazine, quinoline, and pyrimidine);

Ar is substituted with one or more substituents selected from alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido; and

R⁴ is substituted with one or more substituents selected from alkyl, alkenyl, alkynyl, heteroalkyl, cycloalkyl, heterocyclyl, aryl, aralkyl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido.

Exemplary compounds of Formula I and Formula II include:

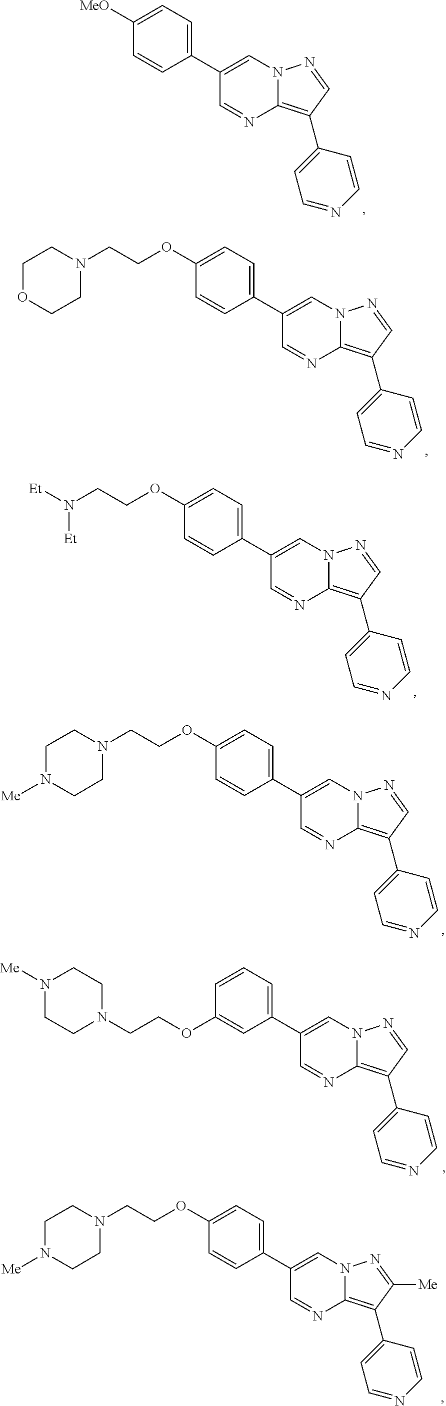 [SMILES: COc1ccc(-c2cnc3c(-c4ccncc4)cnn3c2)cc1 ; c1cc(-c2cnn3cc(-c4ccc(OCCN5CCOCC5)cc4)cnc23)ccn1 ; CCN(CC)CCOc1ccc(-c2cnc3c(-c4ccncc4)cnn3c2)cc1 ; CN1CCN(CCOc2ccc(-c3cnc4c(-c5ccncc5)cnn4c3)cc2)CC1 ; CN1CCN(CCOc2cccc(-c3cnc4c(-c5ccncc5)cnn4c3)c2)CC1 ; Cc1nn2cc(-c3ccc(OCCN4CCN(C)CC4)cc3)cnc2c1-c1ccncc1]

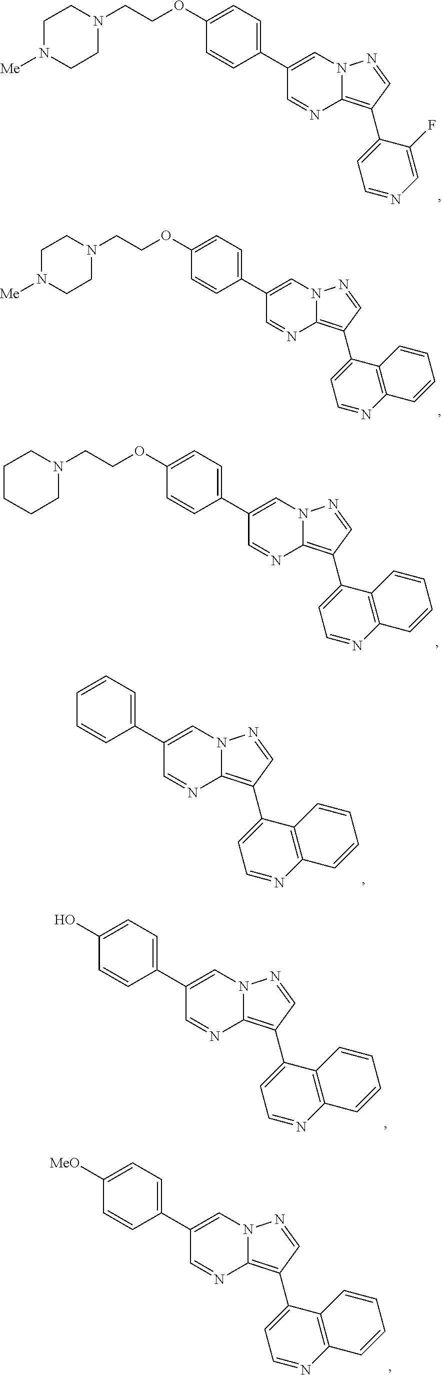 [SMILES: CN1CCN(CCOc2ccc(-c3cnc4c(-c5ccncc5F)cnn4c3)cc2)CC1 ; CN1CCN(CCOc2ccc(-c3cnc4c(-c5ccnc6ccccc56)cnn4c3)cc2)CC1 ; c1ccc2c(-c3cnn4cc(-c5ccc(OCCN6CCCCC6)cc5)cnc34)ccnc2c1 ; c1ccc(-c2cnc3c(-c4ccnc5ccccc45)cnn3c2)cc1 ; Oc1ccc(-c2cnc3c(-c4ccnc5ccccc45)cnn3c2)cc1 ; COc1ccc(-c2cnc3c(-c4ccnc5ccccc45)cnn3c2)cc1]

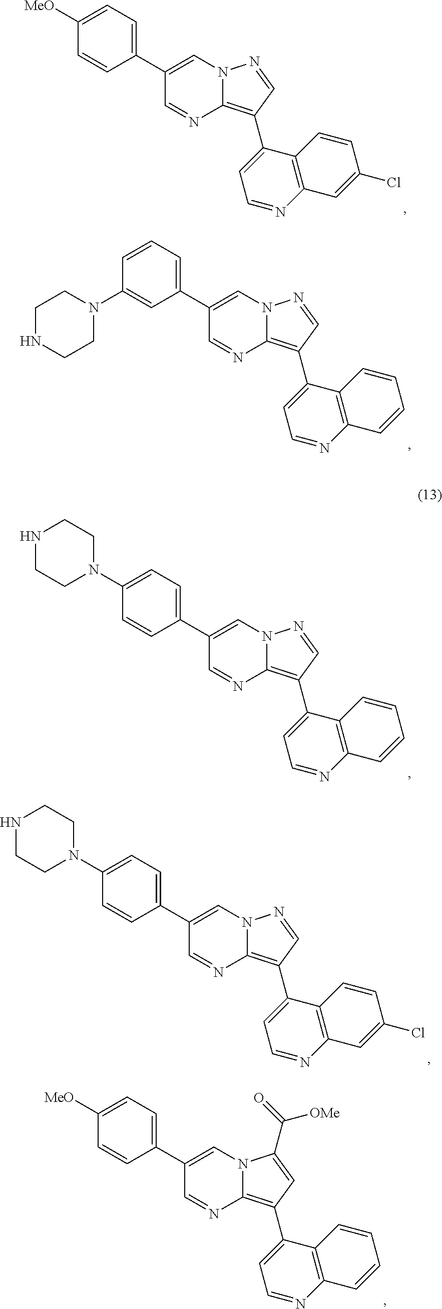

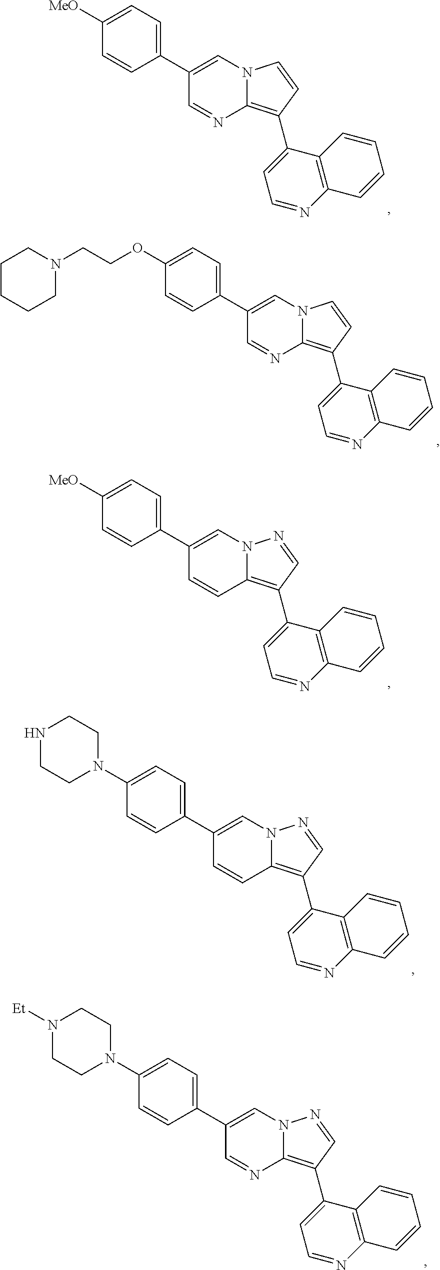 [SMILES: COc1ccc(-c2cnc3c(-c4ccnc5ccccc45)ccn3c2)cc1 ; c1ccc2c(-c3ccn4cc(-c5ccc(OCCN6CCCCC6)cc5)cnc34)ccnc2c1 ; COc1ccc(-c2ccc3c(-c4ccnc5ccccc45)cnn3c2)cc1 ; c1ccc2c(-c3cnn4cc(-c5ccc(N6CCNCC6)cc5)ccc34)ccnc2c1 ; CCN1CCN(c2ccc(-c3cnc4c(-c5ccnc6ccccc56)cnn4c3)cc2)CC1]

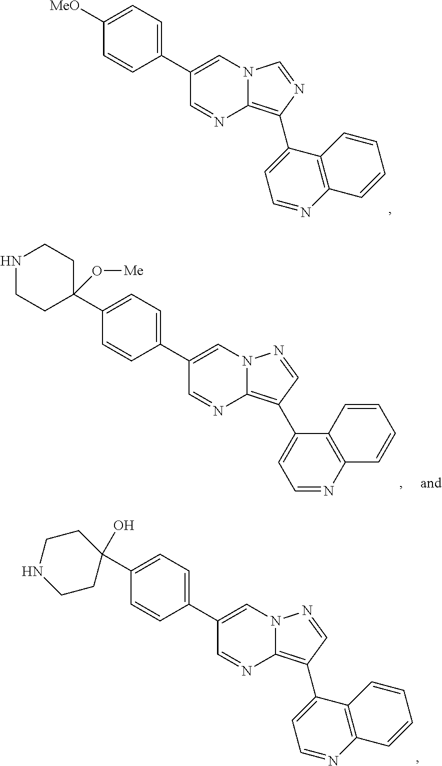 [SMILES: COc1ccc(-c2cnc3c(-c4ccnc5ccccc45)ncn3c2)cc1 ; COC1(c2ccc(-c3cnc4c(-c5ccnc6ccccc56)cnn4c3)cc2)CCNCC1 ; OC1(c2ccc(-c3cnc4c(-c5ccnc6ccccc56)cnn4c3)cc2)CCNCC1]

and salts (including pharmaceutically acceptable salts) of the foregoing.

In one aspect, the invention provides a pharmaceutical composition comprising a compound as disclosed herein and a pharmaceutically acceptable excipient or solvent. In certain embodiments, a pharmaceutical composition may comprise a prodrug of a compound as disclosed herein.

In another aspect, the invention provides a method of inhibiting BMP-induced phosphorylation of SMAD1/5/8, comprising contacting a cell with a compound as disclosed herein. In certain embodiments, the method reduces the circulating levels of ApoB-100 and/or LDL and/or total cholesterol in a subject that has levels of ApoB-100 and/or LDL and/or total cholesterol that are abnormally high or that increase a patient's risk of developing a disease or unwanted medical condition. In certain embodiments, the method of reducing circulating levels of ApoB-100 and/or LDL and/or total cholesterol in a subject reduces the risk of primary or secondary cardiovascular events. In certain embodiments, the method treats or prevents a disease or condition in a subject that would benefit by inhibition of Bone Morphogenetic Protein (BMP) signaling. In certain embodiments, the disease or condition is selected from pulmonary hypertension; hereditary hemorrhagic telangectasia syndrome; cardiac valvular malformations; cardiac structural malformations; fibrodysplasia ossificans progressive; juvenile familial polyposis syndrome; parathyroid disease; cancer (e.g., breast carcinoma, prostate carcinoma, renal cell carcinoma, bone metastasis, lung metastasis, osteosarcoma, and multiple myeloma); anemia; vascular calcification; vascular inflammation; atherosclerosis; acquired or congenital hypercholesterolemia or hyperlipoproteinemia; diseases, disorders, or syndromes associated with defects in lipid absorption or metabolism; diseases, disorders, or syndromes caused by hyperlipidemia; valve calcification; renal osteodystrophy; inflammatory disorders (e.g., ankylosing spondylitis); infections with viruses; bacteria; fungi; tuberculosis; and parasites.

In another aspect, the invention provides a method of treating hypercholesterolemia, hyperlipidemia, hyperlipoproteinemia or hepatic steatosis in a subject comprising administering an effective amount of a compound as disclosed herein. In certain such embodiments, the hypercholesterolemia, hyperlipidemia, hyperlipoproteinemia or hepatic steatosis is acquired hypercholesterolemia, hyperlipidemia, hyperlipoproteinemia or hepatic steatosis. In certain such embodiments, the hypercholesterolemia, hyperlipidemia, hyperlipoproteinemia, or hepatic steatosis is associated with diabetes mellitus, hyperlipidemic diet and/or sedentary lifestyle, obesity, metabolic syndrome, intrinsic or secondary liver disease, biliary cirrhosis or other bile stasis disorders, alcoholism, pancreatitis, nephrotic syndrome, endstage renal disease, hypothyroidism, iatrogenesis due to administration of thiazides, beta-blockers, retinoids, highly active antiretroviral agents, estrogen, progestins, or glucocorticoids.

In another aspect, the invention provides a method of reducing primary and secondary cardiovascular events arising from coronary, cerebral, or peripheral vascular disease in a subject, comprising administering an effective amount of a compound as disclosed herein.

In another aspect, the invention provides a method of preventing and treating hepatic dysfunction in a subject associated with nonalcoholic fatty liver disease (NAFLD), steatosis-induced liver injury, fibrosis, cirrhosis, or non-alcoholic steatohepatitis (NASH) in a subject comprising administering an effective amount of a compound as disclosed herein.

In another aspect, the invention provides a method of inducing expansion or differentiation of a cell, comprising contacting the cell with a compound as disclosed herein. In certain embodiments, the cell is selected from an embryonic stem cell and an adult stem cell. In certain embodiments, the cell is in vitro.

In certain embodiments, a method of the invention may comprise contacting a cell with a prodrug of a compound as disclosed herein.

### BRIEF DESCRIPTION OF THE FIGURES

FIG. 1 shows that the BMP signaling pathway was activated within atherosclerotic lesions in LDL receptor (LDLR)^{-/-} mice on a high fat diet. (a) After six weeks on a high fat diet, atherosclerotic lesions were visible in the aortic minor curvature without signs of calcification (Haematoxylin and Eosin (H/E) staining, top left panel; Arrows mark lesions in the aortic root and lesser curvature); after 20 weeks on a high fat diet, pronounced medial calcification was detectable in the minor curvature (Von Kossa stain, lower right panel; arrows indicate medial calcification in the aortic minor curvature)—all panels frontal, serial sections. Bar indicates 500 μm. (b) Atherosclerotic plaque in the aortic minor curvature of an LDLR^{-/-} mouse on high fat diet for six weeks. Early atheromatous lesions predominantly featured macrophages accumulating beneath the endothelium. Both macrophages and endothelial cells revealed a strong nuclear signal for phosphorylated SMAD1/5/8 (p-SMAD1/5/8). Tissue were stained with antibodies specific for p-SMAD1/5/8 (green) or macrophages (MAC2, red) and counterstained with a DNA-binding dye (DAPI, blue). White bar indicates 100 μm. (c) Treatment with compound 13 for five days suppressed phosphorylation of SMADs 1/5/8 within atherosclerotic lesions in the aortic root of LDLR^{-/-} mice fed a high fat diet for six weeks. Top panels (p-SMAD1/5/8 immunoreactivity, green): vehicle treatment (left) and compound 13 treatment (2.5 mg/kg ip daily, right); lower panels (DAPI staining, blue). White bar indicates 100 μm.

FIG. 2 shows that treatment with BMP antagonists prevented arterial calcification and atherosclerosis in LDLR^{-/-}. (a) Comparison of whole tissue aorta specimens, taken from LDLR^{-/-} mice on high fat diet treated with either vehicle (left panel), or compound 13 (2.5 mg/kg ip daily, right panel) for 20 weeks. Brightfield images (outside) refer to heat maps depicting fluorescence intensity (inside), which represent osteogenic activity based on the uptake of fluor-labeled bisphosphonate (Osteosense 680 nm). Lower panels: Quantified signal intensities in four regions of interest, defined as aortic root (Root), aortic arch (Arch), carotid arteries (Carotids) or thoracic portion of the aorta (Thoracic). Values shown are mean±SEM expressed in arbitrary units (AU, *p≦0.05 vs. corresponding region of interest in vehicle-treated LDLR^{-/-} mice). (b) Pronounced medial calcification was detectable in the minor curvature of vehicle-treated LDLR^{-/-} on high fat diet for 20 weeks, but was significantly less abundant in aortae from LDLR^{-/-} treated with compound 13 (2.5 mg/kg ip daily) based on Alizarin red staining. Frontal sections shown are representative of a total of 20 vehicle- and drug-treated mice. Arrows indicate calcific deposits in the aortic minor curvature. Bar indicates 500 (c) Comparison of whole tissue aorta specimens, taken from LDLR^{-/-} mice on high fat diet treated with either vehicle (left panel), or compound 13 (2.5 mg/kg ip daily, right panel) for 20 weeks. Brightfield images (outside) refer to heat maps depicting fluorescence intensity (inside), which represent macrophage activity based on cathepsin-mediated cleavage of fluor-labeled substrate (Prosense 800 nm). Lower panels: Quantified signal intensities in four regions of interest, defined as aortic Root, Arch, Carotids, or Thoracic aorta. Data are presented as mean±SEM expressed in arbitrary units (AU, *p≦0.05 vs. corresponding region of interest in vehicle treated LDLR^{-/-} mice). (d) Atheroma burden was reduced in LDLR^{-/-} mice treated with compound 13. Representative en face aortae stained with Oil Red O from LDLR^{-/-} mice receiving high fat diet for 20 weeks and treated with vehicle (left) or compound 13 (2.5 mg/kg ip daily, right) are shown from a total of six vehicle- and drug-treated mice. (e) ALK3-Fc inhibited the BMP signaling pathway in vivo. Atherosclerotic lesions in the minor curvature of LDLR^{-/-} mice on high fat diet for 6 weeks and treated with vehicle, ALK3-Fc (2 mg/kg ip every other day), or compound 13 (2.5 mg/kg ip daily) for five days. Top panel: staining with antibodies specific for phosphorylated SMADs 1/5/8 (p-SMAD1/5/8, green). Lower panel: nuclear staining with DAPI (blue). White bar indicates 100 μm. (f) ALK3-Fc and compound 13 inhibited foam cell accumulation in LDLR^{-/-}. Frontal sections of aortic arches from LDLR^{-/-} on high fat diet for 6 weeks and treated with either vehicle, ALK3-Fc (2 mg/kg ip, every other day) or compound 13 (2.5 mg/kg ip, daily) for six weeks. Merged staining for DAPI (blue) and MAC2 (red). White bar indicates 500 μm. (g) ALK3-Fc inhibited macrophage activity in aortas from LDLR^{-/-} mice on high fat diet for six weeks. Signal intensities in four regions of interest, defined as aortic root (Root), aortic arch (Arch), carotid arteries (Carotids) or thoracic aorta (Thoracic) as reflected by cathepsin-mediated cleavage of a near infrared imaging probe. Data presented as mean±SEM, expressed in arbitrary units (AU, *p≦0.05 vs. corresponding region of interest in vehicle-treated LDLR^{-/-} mice).

FIG. 3 shows SMAD1/5/8 phosphorylation was induced within atherosclerotic lesions in LDLR^{-/-} mice on high fat diet. During the first 20 weeks on high fat diet, developing atheromatous lesions revealed strong nuclear staining for phosphorylated SMADs 1/5/8 (p-SMAD1/5/8). Comparison of the minor curvature of LDLR^{-/-} mice on high fat diet for 3, 6, 7, 9 or 20 weeks showed a strong increase in p-SMAD1/5/8 immunoreactivity within the growing atherosclerotic plaque. Left panels: immunofluorescent staining for p-SMAD1/5/8, green. Right panels: Serial sections stained with FITC-labeled secondary antibody only. White bars indicate 500 μm.

FIG. 4 shows the inhibition of BMP signaling reduced induction of reactive oxygen species in endothelial cells. (a) Oxidized LDL- (OxLDL, 80 μg/mL) or BMP2- (20 ng/mL) induced generation of reactive oxygen species (ROS) in human aortic endothelial cells was quantified by chloromethyl 2′,7′-dichlorodihydrofluorescein diacetate (DCF) fluorescence. Induction of ROS by both oxLDL and BMP2 was inhibited by compound 13 (100 nM) or ALK3-Fc (500 ng/mL, *p≦0.05 vs. control without treatment, #p≦0.05 vs. treatment with oxLDL alone, §p≦0.05 vs. treatment with BMP2 alone). (b) BMP2 mRNA was upregulated in human aortic endothelial cells in response to challenge with oxLDL (80 μg/mL) for eight hours (*p≦0.05 vs. control without treatment). oxLDL did not significantly alter expression of genes encoding BMP4, BMP6, BMP7, or BMP9.

FIG. 5 shows that bodyweights and food intake did not differ significantly between mice treated with vehicle and mice treated with compound 13. (a) Mean bodyweight (g) over 20 weeks of high fat diet administration while receiving daily injections of vehicle or compound 13 (2.5 mg/kg ip). (b) Food intake per gram body weight over 6 weeks of high fat diet administration while receiving daily injections of vehicle or compound 13 (2.5 mg/kg ip). Data presented as mean±SEM.

FIG. 6 shows that oxidized LDL induced the production of reactive oxygen species in a dose-dependent manner in human aortic endothelial cells. Human aortic endothelial cells were incubated with the indicated doses of oxidized LDL cholesterol (oxLDL) for 20 hours in EGM-2 media containing 0.1% fetal bovine serum. Cells were incubated for 60 minutes with CM-H₂DCFDA, and fluorescence intensities at 527 nm were measured as a measure of hydrogen peroxide generation. Data presented as mean±SEM. *p≦0.05 versus cells that were not incubated oxidized LDL (0 μg/ml).

FIG. 7 shows that oxLDL-induced generation of reactive oxygen species in human aortic endothelial cells (HAECs), as estimated with lucigenin fluorescence. Induction of ROS by oxLDL could be blocked by incubating cells with compound 13 (100 nM) or ALK3-Fc (500 ng/mL). *p≦0.05 vs. HAECs that were not treated with oxLDL. ^{#}p≦0.05 vs treatment with oxLDL alone.

FIG. 8 shows that BMP2 was induced in HAECs by oxLDL. BMP2 mRNA (a), as measured by quantitative RT-PCR, and BMP2 protein expression (b), as measured by BMP-2 Quantikine ELISA Kit (DBP200, R&D Systems, Minneapolis, Minn.), increased over time in cells incubated with oxidized LDL (80 μg/mL). Data is presented as mean±SEM. *p≦0.05 versus cells not exposed to oxLDL (0 h).

FIG. 9 shows that inhibition of BMP signaling impacted serum lipoprotein and hepatic fat metabolism. (a) Compound 13-treated LDLR^{-/-} mice exhibited lower serum LDL cholesterol levels than did vehicle-treated mice, while HDL cholesterol levels were not altered (*p≦0.05 vs. vehicle treatment, p=ns indicates non-significant). (b) compound 13 did not inhibit HMG-CoA reductase enzyme activity in vitro. Activity assay for 3-hydroxy-3-methylglutaryl-CoA reductase (HMG-CoA reductase), based on spectrophotometric measurement of the decrease in absorbance at 340 nm, in the presence of the substrate HMG-CoA (Control) alone or in the presence of either pravastatin or two different concentrations of compound 13 (50 nM and 100 nM). Data is presented as mean±SEM. (c) Atorvastatin (ATS, 1 nM) and compound 13 (100 nM) reduced basal apolipoprotein B100 (ApoB-100) secretion in HepG2 cells (*p≦0.05 vs. Control). BMP2 (100 ng/mL) induced ApoB-100 secretion (*p≦0.05 vs. Control), which was blocked by incubation with compound 13) but not by incubation with atorvastatin (#p≦0.05 vs. BMP2+compound 13). (d) Compound 13-treated LDLR^{-/-} mice on high fat diet for 20 weeks were protected from hepatic steatosis. Representative sections of hepatic tissue from LDLR^{-/-} mice fed a high fat diet for 20 weeks treated with vehicle (left-hand panels) or compound 13 (2.5 mg/kg ip, daily, right panel panels), shown from a total of 12 vehicle- and drug-treated mice, and stained with haematoxylin and eosin. Bar indicates 400 μm.

FIG. 10 shows that BMP2 induced ApoB-100 production in a time and dose dependent manner in HepG2 cells. (a) After starvation in EMEM culture media containing 0.1% fetal bovine serum for 24 h, HepG2 cells were incubated with BMP2 (100 ng/mL) for varying periods of time. Apolipoprotein B 100 (ApoB) levels, measured in culture medium by ELISA, were increased after 24 h of BMP2 stimulation. Data presented as mean±SEM, n=4, *p≦0.05 vs. control). (b) After starvation in EMEM culture media containing 0.1% fetal bovine serum for 24 h, cells were incubated with the indicated doses of BMP2, and the media was harvested after 24 h. An ELISA was used to determine the amount of secreted ApoB-100 in the supernatant. Data presented as mean±SEM, *p≦0.05 versus cells that were not incubated with BMP2 (0 ng/ml).

FIG. 11 shows that BMP2 (100 ng/mL) induced apolipoprotein B 100 (ApoB-100) secretion in HepG2 cells, which was blocked by either compound 13 (100 nM) or ALK3-Fc (400 ng/mL). *p≦0.05 vs untreated control cells. #p≦0.05 vs BMP2-treated cells in the absence of compound 13 or ALK-Fc.

FIG. 12A shows hematoxylin and eosin stained sections of the aortic root, valve, and aortic arch showing the presence of numerous fibrofatty plaques along the minor curvature of the aortic root and aortic arch in mutant (LDLr−/−) mice prone to hypercholesterolemia, a mouse model of atherosclerosis and athero-calcific vascular disease. These mice were started on a high fat (Paigen) diet at 8 weeks of life, and continued on this diet for 16 weeks to permit the development of atheromatous lesions and vascular calcification.

FIG. 12B shows Von Kossa stained sections of the aortic root, valve, and aortic arch showing intense calcification of the media of the minor curvature of the aortic arch in the mutant (LDLr−/−) mice used in FIG. 12A.

FIG. 13 shows quantitatively that a BMP inhibitor positive control compound can reduce vascular calcification and vascular inflammation in atherogenic mice.

FIG. 14 shows that macrophage-mediated inflammation is quantitatively decreased in the central arterial vascular bed of atherogenic animals by recombinant or small-molecule BMP inhibitors.

FIG. 15A shows an Alizarin stained section of the aorta of a 28 day old wild-type mouse.

FIG. 15B shows an Alizarin stained section of the aorta of a 28 day old MGP−/− mouse.

FIG. 15C shows an Alizarin stained section of the aorta of a 28 day old MGP−/− mouse treated with a BMP inhibitor positive control compound (compound 13); the aorta has less calcification compared to the aorta of the MGP−/− mouse shown in FIG. 15B.

FIG. 15D shows an Alizarin stained section of the aorta of a 28 day old MGP−/− mouse treated with an ALK3-Fc polypeptide; the aorta has less calcification compared to the aorta of the MGP−/− mouse shown in FIG. 15B.

FIG. 16 shows the reduction of arterial calcification, as determined by osteosense fluorescence, in the aorta of MGP−/− mice treated with a BMP inhibitor positive control compound (compound 13) or with an ALK3-Fc polypeptide.

FIG. 17 shows that arterial calcification in MGP−/− mice is associated with excess Smad 1/5/8 phosphorylation (localized in nuclei). Immunohistochemistry of phosphorylated Smad 1/5/8 (A-B), Alizarin red staining for tissue calcium (C-D), and OsteoSense 680 nm imaging (E) of day 28 aortas from vehicle-treated (A, C, left E) and compound 13-treated (B, D, right E) MGP−/− mice are depicted.

FIG. 18 shows the results of experiments revealing that pharmacologic inhibition of BMP signaling reduces aortic calcification in MGP deficiency.

FIG. 19 shows the results of experiments revealing that pharmacologic inhibition of BMP signaling improves survival in MGP deficiency.

FIG. 20 shows that bone mineral density did not differ between LDLR−/− mice treated with vehicle or compound 13.

### DETAILED DESCRIPTION OF THE INVENTION

The invention provides for compounds that inhibit the BMP signaling pathway, as well as methods to treat or prevent a disease or condition in a subject that would benefit by inhibition of BMP signaling.

### I. COMPOUNDS

Compounds of the invention include compounds of Formula I and Formula II as disclosed above. Such compounds are suitable for the compositions and methods disclosed herein. In other embodiments, the following compounds and their salts (including pharmaceutically acceptable salts) are compounds of the invention and are suitable for the compositions and methods disclosed herein:

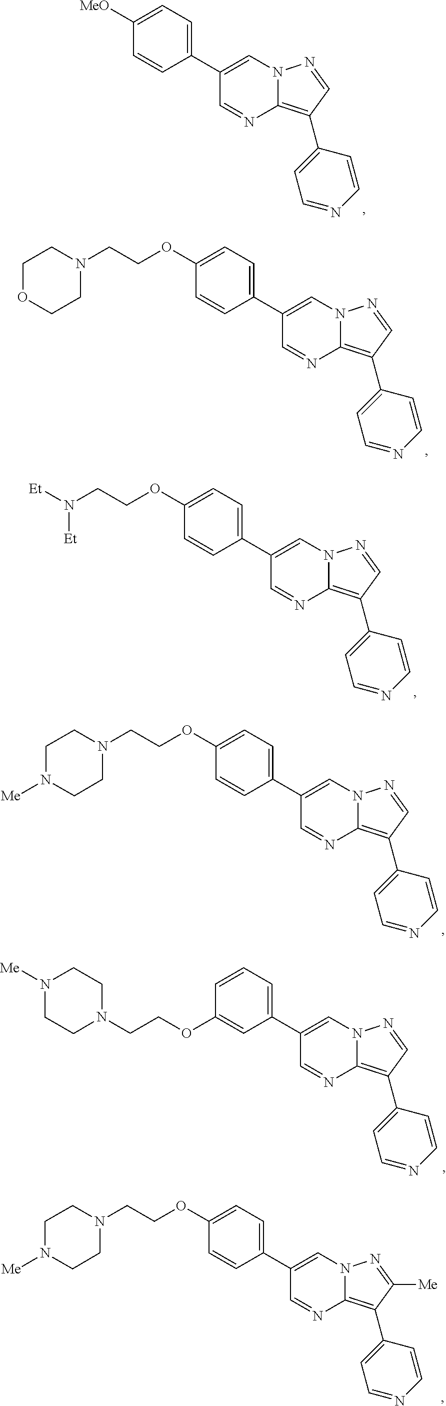 [SMILES: COc1ccc(-c2cnc3c(-c4ccncc4)cnn3c2)cc1 ; c1cc(-c2cnn3cc(-c4ccc(OCCN5CCOCC5)cc4)cnc23)ccn1 ; CCN(CC)CCOc1ccc(-c2cnc3c(-c4ccncc4)cnn3c2)cc1 ; CN1CCN(CCOc2ccc(-c3cnc4c(-c5ccncc5)cnn4c3)cc2)CC1 ; CN1CCN(CCOc2cccc(-c3cnc4c(-c5ccncc5)cnn4c3)c2)CC1 ; Cc1nn2cc(-c3ccc(OCCN4CCN(C)CC4)cc3)cnc2c1-c1ccncc1]

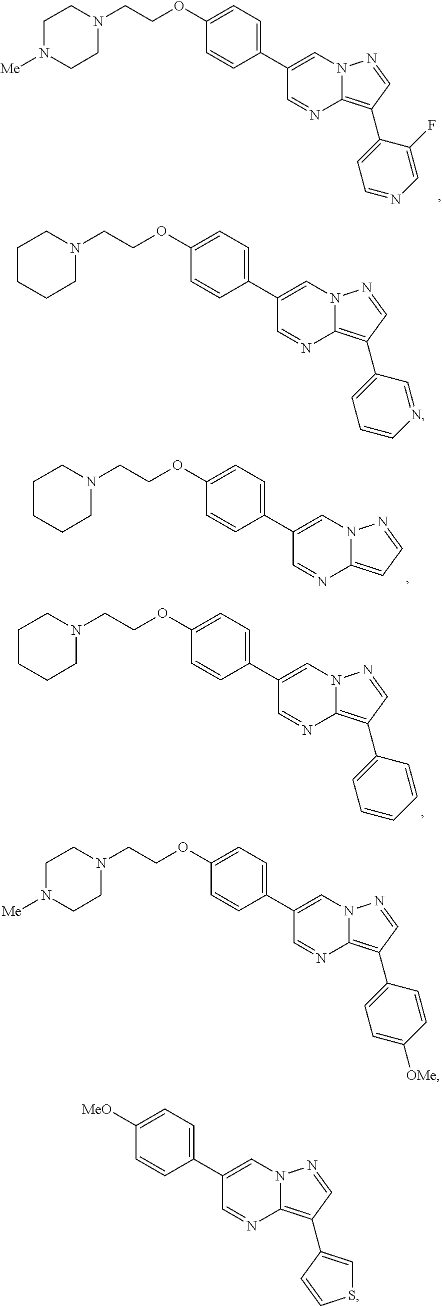 [SMILES: CN1CCN(CCOc2ccc(-c3cnc4c(-c5ccncc5F)cnn4c3)cc2)CC1 ; c1cncc(-c2cnn3cc(-c4ccc(OCCN5CCCCC5)cc4)cnc23)c1 ; c1cc2ncc(-c3ccc(OCCN4CCCCC4)cc3)cn2n1 ; c1ccc(-c2cnn3cc(-c4ccc(OCCN5CCCCC5)cc4)cnc23)cc1 ; COc1ccc(-c2cnn3cc(-c4ccc(OCCN5CCN(C)CC5)cc4)cnc23)cc1 ; COc1ccc(-c2cnc3c(-c4ccsc4)cnn3c2)cc1]

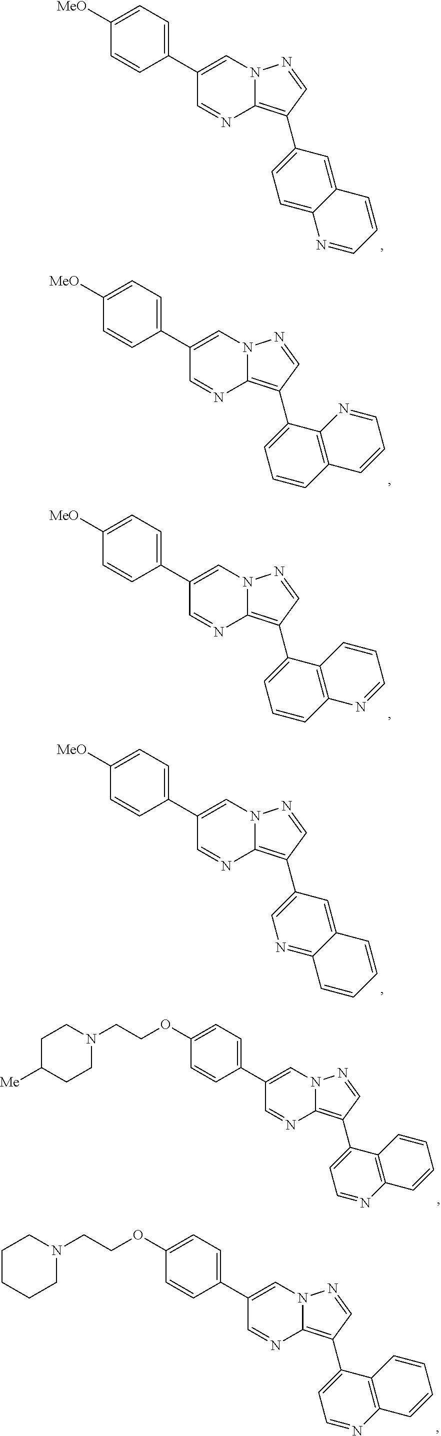 [SMILES: COc1ccc(-c2cnc3c(-c4ccc5ncccc5c4)cnn3c2)cc1 ; COc1ccc(-c2cnc3c(-c4cccc5cccnc45)cnn3c2)cc1 ; COc1ccc(-c2cnc3c(-c4cccc5ncccc45)cnn3c2)cc1 ; COc1ccc(-c2cnc3c(-c4cnc5ccccc5c4)cnn3c2)cc1 ; CC1CCN(CCOc2ccc(-c3cnc4c(-c5ccnc6ccccc56)cnn4c3)cc2)CC1 ; c1ccc2c(-c3cnn4cc(-c5ccc(OCCN6CCCCC6)cc5)cnc34)ccnc2c1]

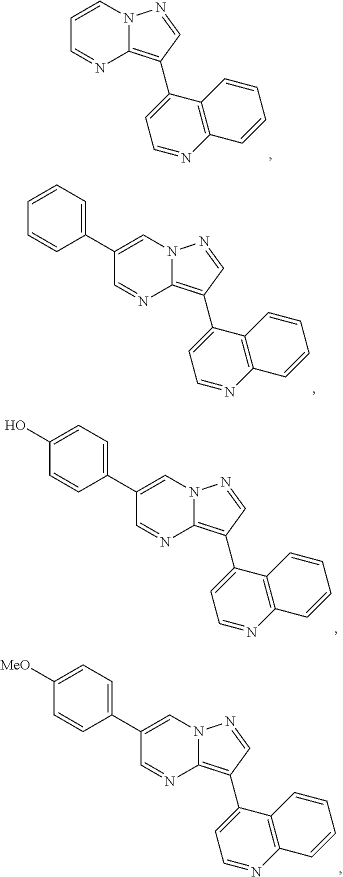 [SMILES: c1ccc2c(-c3cnn4cccnc34)ccnc2c1 ; c1ccc(-c2cnc3c(-c4ccnc5ccccc45)cnn3c2)cc1 ; Oc1ccc(-c2cnc3c(-c4ccnc5ccccc45)cnn3c2)cc1 ; COc1ccc(-c2cnc3c(-c4ccnc5ccccc45)cnn3c2)cc1]

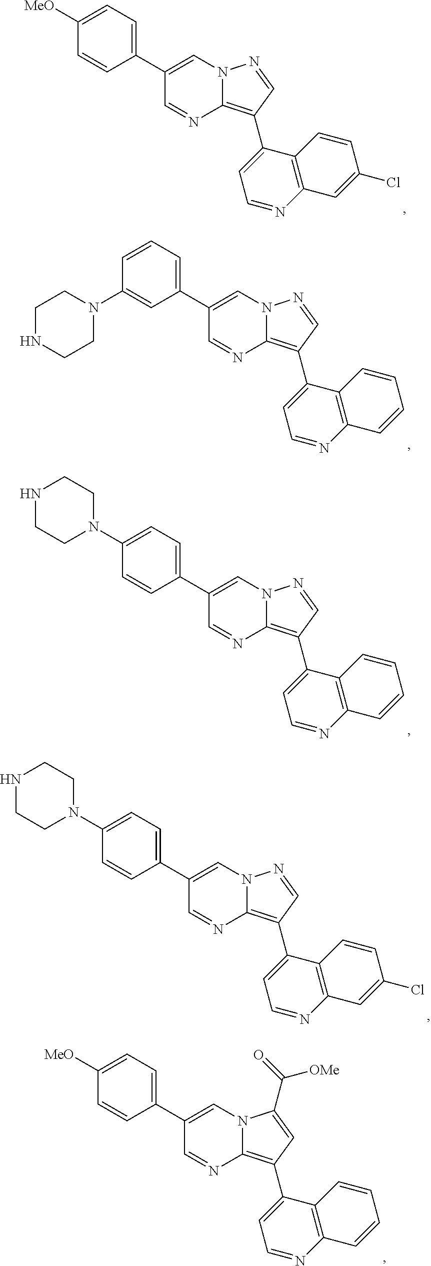

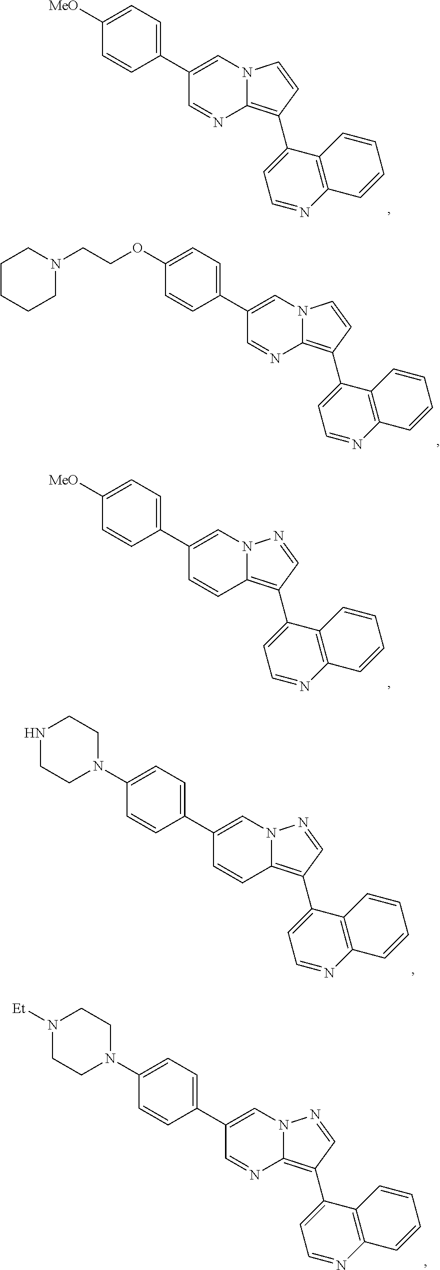 [SMILES: COc1ccc(-c2cnc3c(-c4ccnc5ccccc45)ccn3c2)cc1 ; c1ccc2c(-c3ccn4cc(-c5ccc(OCCN6CCCCC6)cc5)cnc34)ccnc2c1 ; COc1ccc(-c2ccc3c(-c4ccnc5ccccc45)cnn3c2)cc1 ; c1ccc2c(-c3cnn4cc(-c5ccc(N6CCNCC6)cc5)ccc34)ccnc2c1 ; CCN1CCN(c2ccc(-c3cnc4c(-c5ccnc6ccccc56)cnn4c3)cc2)CC1]

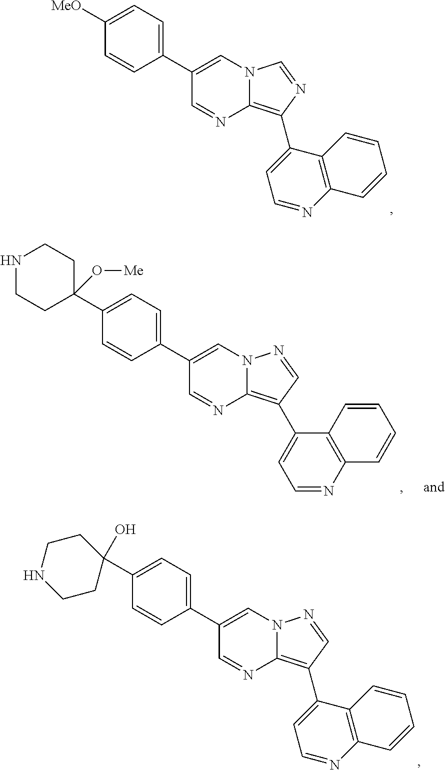 [SMILES: COc1ccc(-c2cnc3c(-c4ccnc5ccccc45)ncn3c2)cc1 ; COC1(c2ccc(-c3cnc4c(-c5ccnc6ccccc56)cnn4c3)cc2)CCNCC1 ; OC1(c2ccc(-c3cnc4c(-c5ccnc6ccccc56)cnn4c3)cc2)CCNCC1]

### II. DEFINITIONS

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—, preferably alkylC(O)NH—.

The term “acyloxy” is art-recognized and refers to a group represented by the general formula hydrocarbylC(O)O—, preferably alkylC(O)O—.

The term “aliphatic”, as used herein, includes straight, chained, branched or cyclic hydrocarbons which are completely saturated or contain one or more units of unsaturation. Aliphatic groups may be substituted or unsubstituted.

The term “alkoxy” refers to an oxygen having an alkyl group attached thereto. Representative alkoxy groups include methoxy, ethoxy, propoxy, tert-butoxy and the like.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated. In preferred embodiments, a straight chain or branched chain alkenyl has 1-12 carbons in its backbone, preferably 1-8 carbons in its backbone, and more preferably 1-6 carbons in its backbone. Exemplary alkenyl groups include allyl, propenyl, butenyl, 2-methyl-2-butenyl, and the like.

The term “alkyl” refers to the radical of saturated aliphatic groups, including straight-chain alkyl groups, and branched-chain alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. In certain embodiments, alkyl groups are lower alkyl groups, e.g. methyl, ethyl, n-propyl, i-propyl, n-butyl and n-pentyl.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. In certain embodiments, a straight chain or branched chain alkyl has 30 or fewer carbon atoms in its backbone (e.g., C₁-C₃₀ for straight chains, C₃-C₃₀ for branched chains). In preferred embodiments, the chain has ten or fewer carbon (C₁-C₁₀) atoms in its backbone. In other embodiments, the chain has six or fewer carbon (C₁-C₆) atoms in its backbone.

Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aryl or heteroaryl moiety.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl^{”} and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated. In preferred embodiments, an alkynyl has 1-12 carbons in its backbone, preferably 1-8 carbons in its backbone, and more preferably 1-6 carbons in its backbone. Exemplary alkynyl groups include propynyl, butynyl, 3-methylpent-1-ynyl, and the like.

The term “amide”, as used herein, refers to a group

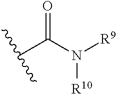

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

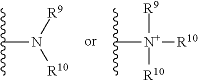

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with one or more aryl groups.

The term “aryl”, as used herein, include substituted or unsubstituted single-ring aromatic groups in which each atom of the ring is carbon. Preferably the ring is a 5- to 7-membered ring, more preferably a 6-membered ring. Aryl groups include phenyl, phenol, aniline, and the like.

The term “carbamate” is art-recognized and refers to a group

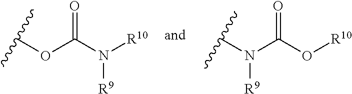

wherein R⁹ and R¹⁰ independently represent hydrogen or a hydrocarbyl group, such as an alkyl group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R⁹, wherein R⁹ represents a hydrocarbyl group, such as an alkyl group.

The term “carboxy”, as used herein, refers to a group represented by the formula −CO₂H.

The term “cycloalkyl”, as used herein, refers to the radical of a saturated aliphatic ring. In preferred embodiments, cycloalkyls have from 3-10 carbon atoms in their ring structure, and more preferably from 5-7 carbon atoms in the ring structure. Suitable cycloalkyls include cycloheptyl, cyclohexyl, cyclopentyl, cyclobutyl and cyclopropyl.

The term “ester”, as used herein, refers to a group —C(O)OR⁹ wherein R⁹ represents a hydrocarbyl group, such as an alkyl group or an aralkyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen”, as used herein, means halogen and includes chloro, fluoro, bromo, and iodo.

The term “heteroalkyl”, as used herein, refers to a saturated or unsaturated chain of carbon atoms including at least one heteroatom (e.g., O, S, or NR⁵⁰, such as where R⁵⁰ is H or lower alkyl), wherein no two heteroatoms are adjacent.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom (e.g., 0, N, or S), preferably one to four or one to 3 heteroatoms, more preferably one or two heteroatoms. When two or more heteroatoms are present in a heteroaryl ring, they may be the same or different. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Preferred polycyclic ring systems have two cyclic rings in which both of the rings are aromatic. Heteroaryl groups include, for example, pyrrole, furan, thiophene, imidazole, oxazole, thiazole, pyrazole, pyridine, pyrazine, pyridazine, quinoline, and pyrimidine, and the like.

The term “heteroatom”, as used herein, means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. Examples of straight chain or branched chain lower alkyl include methyl, ethyl, isopropyl, propyl, butyl, tertiary-butyl, and the like. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitation aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Preferred polycycles have 2-3 rings. Each of the rings of the polycycle can be substituted or unsubstituted. In certain embodiments, each ring of the polycycle contains from 3 to 10 atoms in the ring, preferably from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of the invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt or ester thereof.

The term “sulfonamide” is art-recognized and refers to the group represented by the general formulae

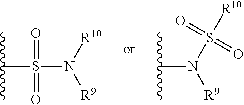

wherein R⁹ and R¹⁰ independently represents hydrogen or hydrocarbyl, such as alkyl.

The term “sulfoxide” is art-recognized and refers to the group —S(O)—R⁹, wherein R⁹ represents a hydrocarbyl, such as alkyl, aryl, or heteroaryl.

The term “sulfonate” is art-recognized and refers to the group —SO₃H, or a pharmaceutically acceptable salt or ester thereof.

The term “sulfone” is art-recognized and refers to the group —S(O)₂—R⁹, wherein R⁹ represents a hydrocarbyl, such as alkyl, aryl, or heteroaryl.

The term “thioester”, as used herein, refers to a group —C(O)SR⁹ or —SC(O)R⁹ wherein R⁹ represents a hydrocarbyl, such as alkyl.

The term “thioether”, as used herein, is equivalent to an ether, wherein the oxygen is replaced with a sulfur.

The term “urea” is art-recognized and may be represented by the general formula

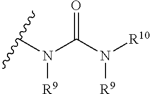

wherein R⁹ and R¹⁰ independently represent hydrogen or a hydrocarbyl, such as alkyl.

At various places in the present specification substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, propyl, isopropyl, n-butyl, sec-butyl, isobutyl, etc.

For a number qualified by the term “about”, a variance of 2%, 5%, 10% or even 20% is within the ambit of the qualified number

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “prodrug” is intended to encompass compounds which, under physiologic conditions, are converted into the therapeutically active agents of the present invention (e.g., a compound of Formula I or Formula II). A common method for making a prodrug is to include one or more selected moieties which are hydrolyzed under physiologic conditions to reveal the desired molecule. In other embodiments, the prodrug is converted by an enzymatic activity of the host animal. For example, esters (e.g., esters of alcohols or carboxylic acids) are preferred prodrugs of the present invention. In various embodiments disclosed herein (e.g., the various compounds, compositions, and methods), some or all of the compounds of formula A, compounds of any one of Formula I or Formula II, all or a portion of a compound of Formula I or Formula II in a formulation represented above can be replaced with a suitable prodrug, e.g., wherein a hydroxyl or carboxylic acid present in the parent compound is presented as an ester.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

### III. PHARMACEUTICAL COMPOSITIONS

Compounds of the present invention may be used in a pharmaceutical composition, e.g., combined with a pharmaceutically acceptable carrier, for administration to a patient. Such a composition may also contain diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. Such additional factors and/or agents may be included in the pharmaceutical composition to produce a synergistic effect with compounds of the invention, or to minimize side effects caused by the compound of the invention.

The pharmaceutical compositions of the invention may be in the form of a liposome or micelles in which compounds of the present invention are combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The terms “pharmaceutically effective amount” or “therapeutically effective amount”, as used herein, means the total amount of each active component of the pharmaceutical composition or method that is sufficient to show a meaningful patient benefit, e.g., treatment, healing, prevention, inhibition or amelioration of a physiological response or condition, such as an inflammatory condition or pain, or an increase in rate of treatment, healing, prevention, inhibition or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

Each of the methods of treatment or use of the present invention, as described herein, comprises administering to a mammal in need of such treatment or use a pharmaceutically or therapeutically effective amount of a compound of the present invention, or a pharmaceutically acceptable salt or ester form thereof. Compounds of the present invention may be administered in accordance with the method of the invention either alone or in combination with other therapies.

Administration of compounds of the present invention used in the pharmaceutical composition or to practice the method of the present invention can be carried out in a variety of conventional ways, such as oral ingestion, inhalation, or cutaneous, subcutaneous, or intravenous, intramuscular, and intraperitoneal injection.

When a therapeutically effective amount of a compound(s) of the present invention is administered orally, compounds of the present invention may be in the form of a tablet, capsule, powder, solution or elixir. When administered in tablet form, the pharmaceutical composition of the invention may additionally contain a solid carrier such as a gelatin or an adjuvant. The tablet, capsule, and powder may contain from about 5 to 95% compound of the present invention, and preferably from about 10% to 90% compound of the present invention. When administered in liquid form, a liquid carrier such as water, petroleum, oils of animal or plant origin such as peanut oil, mineral oils, phospholipids, tweens, triglycerides, including medium chain triglycerides, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition typically contains from about 0.5 to 90% by weight of compound of the present invention, and preferably from about 1 to 50% compound of the present invention.

When a therapeutically effective amount of a compound(s) of the present invention is administered by intravenous, cutaneous or subcutaneous injection, compounds of the present invention may be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, cutaneous, or subcutaneous injection should contain, in addition to compounds of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of compound(s) of the present invention in the pharmaceutical composition of the present invention will depend upon the nature and severity of the condition being treated, and on the nature of prior treatments the patient has undergone. Ultimately, the practitioner will decide the amount of compound of the present invention with which to treat each individual patient. Initially, the practitioner may administer low doses of compound of the present invention and observe the patient's response. Larger doses of compounds of the present invention may be administered until the optimal therapeutic effect is obtained for the patient, and at that point the dosage is not increased further. It is contemplated that the various pharmaceutical compositions used to practice the method of the present invention should contain about 0.1 μg to about 100 mg (preferably about 0.1 mg to about 50 mg, more preferably about 1 mg to about 2 mg) of compound of the present invention per kg body weight.

The duration of intravenous therapy using the pharmaceutical composition of the present invention will vary, depending on the severity of the disease being treated and the condition and potential idiosyncratic response of each individual patient. It is contemplated that the duration of each application of the compounds of the present invention will be in the range of 12 to 24 hours of continuous intravenous administration. Ultimately the practitioner will decide on the appropriate duration of intravenous therapy using the pharmaceutical composition of the present invention.

### IV. USE WITH POLYMERS

The compounds as disclosed herein may be conjugated to a polymer matrix, e.g., for controlled delivery of the compound. The compound may be conjugated via a covalent bond or non-covalent association. In certain embodiments wherein the compound is covalently linked to the polymer matrix, the linkage may comprise a moiety that is cleavable under biological conditions (e.g., ester, amide, carbonate, carbamate, imide, etc.). In certain embodiments, the conjugated compound may be a pharmaceutically acceptable salt, ester, or prodrug of a compound disclosed herein. A compound as disclosed herein may be associated with any type of polymer matrix known in the art for the delivery of therapeutic agents.

### V. SYNTHETIC PREPARATION

The compounds disclosed herein can be prepared in a variety of ways known to one skilled in the art of organic synthesis, and in analogy with the exemplary compounds whose synthesis is described herein. The starting materials used in preparing these compounds may be commercially available or prepared by known methods. Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

### VI. USES

BMPs and TGF-beta signaling pathways are essential to normal organogenesis and pattern formation, as well as the normal and pathological remodeling of mature tissues. Defects in the BMP signaling pathway are implicated in a number of congenital and acquired disease processes, including Hereditary Hemorrhagic Telangectasia syndrome, Primary Pulmonary Hypertension, Juvenile Familial Polyposis, as well as sporadic renal cell and prostate carcinomas. It has been suggested that in certain disease states associated with defective signaling components, attenuated BMP signaling might be a cause, while our findings have suggested that in some contexts excess BMP signaling might be pathogenic (Waite et al. Nat. Rev. Genet. 4:763-773, 2005; Yu et. J. Biol. Chem. 280:24443-24450, 2003). The ability to modulate BMP signaling experimentally would provide a means for investigating therapy, and for determining the root causes of these conditions.

A. Treatment of Anemia, Including Iron Deficiency and Anemia of Chronic Disease

For a review, see Weiss et al. N. Engl. J. Med. 352:1011-1023, 2005. Anemia of inflammation (also called anemia of chronic disease) can be seen in patients with chronic infections, autoimmune diseases (such as systemic lupus erythematosis and rheumatoid arthritis, and Castleman's disease), inflammatory bowel disease, cancers (including multiple myeloma), and renal failure. Anemia of inflammation is often caused by maladaptive expression of the peptide hormone hepcidin. Hepcidin causes degradation of ferroportin, a critical protein that enables transport of iron from intracellular stores in macrophages and from intestinal epithelial cells. Many patients with renal failure have a combination of erythropoietin deficiency and excess hepcidin expression. BMP signaling induces expression of hepcidin and inhibiting hepcidin expression with BMP antagonists increases iron levels. Compounds as described herein can be used to treat anemia due to chronic disease or inflammation and associated hyperhepcidinemic states.

The inflammatory cytokine IL-6 is thought to be the principal cause of elevated hepcidin expression in inflammatory states, based upon the elevation of IL-6 in anemia of inflammation of diverse etiologies, the effects of chronic IL-6 administration in vivo, and the protection against anemia in rodents deficient in IL-6 (Weiss et al. N. Engl. J. Med. 352:1011-1023, 2005). It has been shown that stimulating hepatoma cell lines with IL-6 induces hepcidin expression, while treatment with a BMP antagonist abrogates IL-6-induced hepcidin expression (Yu et al. Nat. Chem. Biol. 4:33-41, 2008). Moreover, we have found that BMP antagonists can inhibit hepcidin expression induced by injection of pathogenic bacteria in vivo (see Example 8). It has also been shown that systemic iron administration in mice and zebrafish rapidly activates BMP-responsive-SMADs and hepcidin expression in the liver, and that BMP antagonism effectively blocks these responses (Yu et al. Nat. Chem. Biol. 4:33-41, 2008). The functional importance of BMP signaling in iron regulation is supported by our finding that BMP antagonists can inhibit hepcidin expression and raise serum iron levels in vivo (see Example 7). Taken together these data suggest that iron- and inflammation-mediated regulation of hepcidin and circulating iron levels require BMP signaling. Compounds as described herein may be used to alter iron availability in diverse circumstances for therapeutic benefit.

Compounds as described herein may be used in anemic states to (i) augment the efficacy of dietary iron or oral iron supplementation (which is safer than intravenous administration of iron) to increase serum iron concentrations; (ii) augment build up of hemoglobin in the blood in anticipation of surgery or to enable blood donation for self in anticipation of surgery; and (iii) enhance the efficacy of erythropoietin and its relatives, thereby enabling lower doses of erythropoietin to be administered for anemia while minimizing known toxicities and side effects of erythropoietin (i.e., hypertension, cardiovascular events, and tumor growth).

B. Treatment of Fibrodysplasia Ossificans Progressiva (FOP)

FOP is caused by the presence of a constitutively-active mutant form of ALK2 in affected individuals (Shore et al. Nat. Genet. 38:525-527, 2006). A specific inhibitor of BMP signaling such as a compound as described herein can be used to prevent excessive bone formation in response to trauma, musculoskeletal stress or inflammation. Such a compound could also be used to aid in regression of pathologic bone. The BMP inhibitor could be administered systemically or locally to concentrate or limit effects to areas of trauma or inflammation.

A BMP inhibitor as described herein may be used as chronic therapy to suppress spontaneous bone formation in individuals who are highly susceptible. Transient therapy may be used to prevent abnormal bone formation in FOP individuals who develop osteomas or pathologic bone most frequently in association with trauma by administration before, during, or even after the traumatic incident. Transient therapy with BMP inhibitors as described herein could be used before, during or immediately after necessary or emergent medical or surgical procedures (and even important immunizations and tooth extractions) in individuals with FOP, to prevent pathologic calcification. Combination therapy with other bone inhibiting agents, immune modulatory or anti-inflammatory drugs (such as NSAIDs, steroids, cyclosporine, cyclophosphamide, azathioprine, methotrexate, rituxumab, etanercept, or similar drugs) may increase the effectiveness of BMP antagonists in inhibiting heterotopic bone formation in this disorder.

A mouse model of FOP has been developed in which expression of a constitutively-active mutant form of ALK2 is induced by injecting the popliteal fossa of a genetically-modified mouse with an adenovirus directing expression of Cre recombinase. This model reproduces the ectopic calcification and disability seen in FOP patients. Twice daily administration of compound 13 (3 mg/kg ip) prevented the ectopic calcification and disability (see Example 10).

C. Treatment of Cancers

Excessive BMP signaling, which could arise due to over-expression of BMPs, or, paradoxically, as a result of loss of BMP type II receptor expression, may contribute to the oncogenesis, growth or metastasis of certain solid tumors, including breast, prostate carcinomas, bone, lung, and renal cell carcinomas (Yu et al. J. Biol. Chem. 280:24443-24450, 2008; Waite et al. Nat. Rev. Genet. 4:763-773, 2003; Alarmo et al. Genes, Chromosomes Cancer 45:411-419, 2006; Kim et al. Cancer Res. 60:2840-2844, 2000; Kim et al. Clin. Cancer Res. 9:6046-6051, 2003; Kim et al. Oncogene 23:7651-7659, 2004). If increased BMP activity associated with BMP over-expression or BMP type II receptor deficiency contributes to the pathogenesis of disease, then inhibiting BMP signaling activity using compounds as described herein at the level of BMP type I receptors (downstream of both ligands and type II receptor) could be an effective means of normalizing BMP signaling activity and potentially inhibiting tumor growth or metastasis.

Compounds as described herein can be used to slow or arrest the growth or metastasis of such tumor cells (as well as other tumor constituent cell types) for clinical benefit, either as adjunctive or primary chemotherapy. Also, BMP inhibitors as described herein may be used to interfere with the bone metastatic properties of certain types of cancers (e.g., adenocarcinoma, such as prostate and breast carcinomas). In addition, compounds as described herein can be used to inhibit osteoblastic activity in tumors that either form bone or are bone-derived, such as osteosarcomas (as adjunctive or primary chemotherapy). Further, compounds as described herein can be used to inhibit osteoclastic activity (also regulated by BMPs through the action of its target gene RANKL), which is pathologically increased in conditions such as multiple myeloma and other bone-targeted tumors. Application of BMP inhibitors in these conditions may reduce the presence of osteolytic lesions and bone fractures due to tumor involvement.

D. Immune Modulation Via BMP Antagonists

BMPs have been reported to attenuate the inflammatory or immune response (Choi et al. Nat. Immunol. 7:1057-1065, 2006; Kersten et al. BMC Immunol. 6:9, 2005), which can impair an individual's ability to fight infections (i.e., viral, bacterial, fungal, parasitic, or tuberculosis). Inhibitors of BMP signaling as described herein may thus augment the inflammatory or immune response enabling individuals to clear infections more rapidly.

Lymphocytes and other immune cells express BMP receptors on their cell surfaces, and there is growing evidence that BMPs regulate the development and maturation of various humoral and cellular immunologic compartments, and regulate humoral and cellular immune responses in mature organisms. The effects of BMP signals on immune cells are likely to be context-specific, as is commonly known for the effects of numerous cytokines of immunologic importance, and thus whether they augment or diminish the development or function of particular lymphocyte populations must be empirically determined. BMP antagonism using compounds as described herein may be an effective strategy for intentionally biasing the development of cellular, innate, or humoral immune compartments for therapy, or a strategy for the therapeutic deviation of immune responses in mature immune systems. These strategies may target inborn disorders of cellular, innate, or humoral immunity, or target disorders in which immune responses are inappropriately weak (e.g., as an adjuvant to promote successful antigen sensitization when immunization is difficult or ineffective by other means), or target disorders in which immune responses are excessive or inappropriate (e.g., autoimmunity and autosensitization). BMP antagonists as described herein may also be effective in some contexts for the intentional induction of immune tolerance (i.e., in allotransplantation or autoimmunity).

E. Treatment of Pathologic Bone Formation

Compounds as described herein can be used to ameliorate pathologic bone formation/bone fusion in inflammatory disorders, such as ankylosing spondylitis or other “seronegative” spondyloarthropathies, in which autoimmunity and inflammation in such disorders appear to stimulate bone formation. One application of the compounds would be to prevent excess bone formation after joint surgery, particularly in patients with ankylosing spondylitis or rheumatoid arthritis. Compounds as described herein can also be used to prevent calcinosis (dystrophic soft-tissue calcification) in diseases such as systemic lupus erythematosus, scleroderma, or dermatomyositis.

Blunt traumatic injury to muscles can cause abnormal bone formation within muscle in certain individuals, resulting in a disorder called myositis ossificans traumatica (Cushner et al. Orthop. Rev. 21:1319-1326, 1992.). Head trauma and burn injury can also induce heterotopic bone formation markedly impairing patient rehabilitation and recovery. Treatment with a BMP inhibitor as described herein, optionally in addition to anti-inflammatory medications usually prescribed for such a condition (eg. non-steroidal anti-inflammatory drugs such as indomethacin or ibuprofen) may help to prevent the formation of pathologic bone in predisposed individuals, or to help lessen or regress lesions in individuals recently or remotely affected. Very rarely other muscles have been described to develop ossification in the presence of injury or trauma, including heart muscle, and similar treatment with a BMP inhibitor as described herein could be helpful in those circumstances.

F. Treatment of Ectopic or Maladaptive Bone Formation

BMP signals and their transcriptional targets are implicated in intimal and medial vascular remodeling and calcification in Monckeberg's vascular calcification disease and in atheromatous vascular disease (Bostrom et al. J. Clin. Invest. 91:1800-1809, 1993; Tyson et al. Arterioscler. Thromb. Vasc. Biol. 23:489-494, 2003). BMPs and BMP-induced osteodifferentation are also implicated in cardiac valvular calcification. Native cardiac valves can calcify particularly when they are already abnormal. A classic example is bicuspid aortic valve—these valves typically become calcified leading to stenosis. Patients with calcific aortic valve stenosis often require cardiac surgery for valve replacement. Abnormal calcification can adversely affect the function of prosthetic vascular grafts or cardiac valves. For example, prosthetic heart valves become calcified leading to narrowing and often leakage.

Compounds as described herein can be used to inhibit vascular or valvular calcific disease alone or in combination with atheromatous disease, renal disease, renal osteodystrophy or parathyroid disease.

Compounds as described herein can be used to inhibit calcification of prosthetic vascular or valvular materials by systemic or local administration or direct incorporation into prosthesis materials or other implants (e.g., in admixture with a polymer that coats or constitutes all or part of the implant or prosthesis).

In some instances, it is desired to delay fracture healing following a bone fracture, or to purposely inhibit fracture healing in certain locations to prevent impairment of function by maladaptive bone formation. For example, if a fracture occurs and for medical or practical reasons surgery cannot be performed immediately, fracture healing may be temporarily “suspended” by use of a BMP inhibitor as described herein, until definitive surgery or manipulation can be performed. This could prevent the need for subsequent intentional re-fracture in order to ensure correct apposition of bone fragments, for example. It is expected that upon stopping a BMP inhibitor normal fracture healing processes would ensue if the period of treatment is relatively short. In other cases, any amount of novel bone growth might impair function, such as when fracture affects a joint directly. In these cases, global or local inhibition of BMP activity (by systemic or local delivery of a BMP antagonist as described herein via diffusion from a local implant or matrix) may be used to inhibit fracture healing or prevent fracture calluses at the critical areas.

G. Treatment of Skin Diseases

Expansion of cultured keratinocytes—In vitro, BMPs inhibit keratinocyte proliferation and promote differentiation (reviewed in Botchkarev et al. Differentiation 72:512-526, 2004). In patients in need of skin grafting (eg. after burns), skin grafts are made from cultured keratinocytes. The keratinocytes may be derived from other animals (xenografts), but these are only temporary as they will be rejected by the immune system. Keratinocytes can be derived from the patient themselves and can be grown into sheets of cells in the laboratory (cultured epithelial autografts). The patient will not reject keratinocytes derived from his/her own body. Addition of BMP antagonists as described herein to keratinocyte cultures can be used to facilitate keratinocyte proliferation enabling patients to receive grafts sooner.

Improved epithelialization—BMP6 is highly expressed in skin injury, and high levels of BMP6 are detected in chronic human wounds of different etiologies (Kaiser et al. J. Invest. Dermatol. 111:1145-1152, 1998). In mice overexpressing BMP6 in their skin, reepithelialization and healing skin wounds were significantly delayed (Kaiser et al. J. Invest. Dermatol. 111:1145-1152, 1998). Improved epithelialization can reduce scar formation. Topical or systemic administration of BMP antagonists as described herein can be used to augment epithelialization of skin wounds, for example, in the treatment of pressure ulcers (bed sores) or non-healing or poorly-healing skin ulcers (e.g., in patients with peripheral vascular disease, diabetes mellitus, venous incompetence). Compounds would also be expected to decrease scar formation.

Promotion of hair growth—Growth of hair follicles on the scalp is cyclic with three phases: anagen (the growth phase), catagen (the involutional phase), and telogen (resting phase). Recent evidence suggests that BMP signals delay the transition from telogen to anagen (Plikus et al. Nature 451:340-344, 2008). Inhibition of BMP signaling using compounds as described herein can shorten the telogen phase and increase the number of follicles in the anagen phase. Compounds as described herein can be used to treat circumstances wherein hair follicles are insufficient or when hairs are being lost more frequently than they are grown. These circumstances include androgenetic alopecia (male pattern balding), alopecia areata, and telogen effluvium.

Treatment of psoriasis—Psoriasis is an inflammatory skin disorder which sometimes occurs following skin trauma and the ensuing repair and inflammation (Koebner phenomenon). BMPs may participate in repair and inflammatory mechanisms that cause psoriasis, since over-expression of BMP6 in the skin of mice leads to skin lesions similar to those seen in patients with psoriasis (Blessing et al. J. Cell. Biol. 135:227-239, 1996). Compounds as described herein may be administered topically or systemically to treat established psoriasis or prevent its development after skin injury.

Treatment of corneal scarring—BMP6 expression is associated with conjunctival scarring (Andreev et al. Exp. Eye Res. 83:1162-1170, 2006). Compounds as described herein can be used to prevent or treat corneal scarring and the resulting blindness.

H. Treatment of Systemic Hypertension

Infusion of BMP4 induces systemic hypertension in mice (Miriyala et al. Circulation 113:2818-2825, 2006). Vascular smooth muscle cells express a variety of BMP ligands. BMPs increase the expression of voltage gated potassium channels and thereby increase constriction of vascular smooth muscle (Fantozzi et al. Am. J. Physiol. Lung Cell. Mol. Physiol. 291:L993-1004, 2006). Compounds as described herein that inhibit BMP signaling can be used to reduce blood pressure. Sustained reduction of blood pressure in patients with hypertension would be expected to prevent myocardial infarction, congestive heart failure, cerebrovascular accidents, and renal failure. BMP inhibitors as described herein can be used to target the hypertension in specific vascular beds, such as in pulmonary hypertension via local delivery (e.g., via aerosol).

I. Treatment of Pulmonary Hypertension

BMP signaling contributes to the pathogenesis of pulmonary hypertension. For example, mice with decreased BMP4 levels are protected from the pulmonary hypertension and pulmonary vascular remodeling induced by breathing low oxygen concentrations for prolonged periods (Frank et al. Circ. Res. 97:496-504, 2005). Moreover, mutations in the gene encoding the type II BMP receptor (BMPRII) are frequently found in patients with sporadic and familial pulmonary arterial hypertension. It might be anticipated that decreased BMP signaling might cause pulmonary hypertension. However, Yu and colleagues (Yu et al. J. Biol. Chem. 280:24443-24450, 2008) reported that BMPRII deficiency paradoxically increases BMP signaling by subsets of BMP ligands, and thus increased BMP signaling using compounds as described herein may actually contribute to the development of pulmonary hypertension.

Compounds as described herein can used to prevent the development of pulmonary arterial hypertension in patients at risk for the disease (e.g., patients with BMPRII mutations) or to treat patients with idiopathic or acquired pulmonary arterial hypertension. Decreased pulmonary hypertension in individuals treated with the compounds described herein would be expected to decrease shortness of breath, right ventricular hypertrophy, and right ventricular failure.

J. Treatment of Ventricular Hypertrophy

BMP-10 levels are increased in the hypertrophied ventricles of rats with hypertension, and this BMP ligand induces hypertrophy in cultured neonatal rat ventricular myocytes (Nakano et al. Am. J. Physiol. Heart. Circ. Physiol. 293:H3396-3403, 2007). Inhibition of BMP-10 signaling with compounds as described herein can to prevent/treat ventricular hypertrophy. Ventricular hypertrophy can lead to congestive heart failure due to diastolic dysfunction. Compounds described herein would be expected to prevent/treat congestive heart failure.

K. Treatment of Neurologic Disorders

Treatment of spinal cord injury and neuropathy—BMPs are potent inhibitors of axonal regeneration in the adult spinal cord after spinal cord injury (Matsuura et al. J. Neurochem. 2008). Expression of BMPs is reported to be elevated in oligodendrocytes and astrocytes around the injury site following spinal cord contusion. Intrathecal administration of noggin, a BMP inhibitor, led to enhanced locomotor activity and significant regrowth of the corticospinal tract after spinal cord contusion.

RGMa inhibits axonal growth and recovery after spinal cord injury, as well as synapse re-formation, effects which are blocked by an antibody directed against RGMa (Hata et al. J. Cell. Biol. 173:47-58, 2006; Kyoto et al. Brain Res. 1186:74-86, 2007). RGMa enhances BMP signaling (Babitt et al. J. Biol. Chem. 280:29820-29827, 2005) suggesting that BMP signaling may be responsible for preventing axonal growth and recovery.

Based on these considerations, compounds as described herein would be expected to increase axonal growth and recovery after spinal cord injury. Compounds as described herein would be expected to prevent/treat neuropathies associated with a wide spectrum of disorders including diabetes mellitus. Compounds as described herein would be expected to treat both the pain and motor dysfunction associated with neuropathies.

Treatment of neurologic disorders associated with central nervous system inflammation—BMP4 and 5 have been detected in multiple sclerosis and Creutzfeldt-Jakob disease lesions (Deininger et al. Acta Neuropathol. 90:76-79, 1995). BMPs have also been detected in mice with experimental autoimmune encephalomyelitis, an animal model of multiple sclerosis (Ara et al. J. Neurosci. Res. 86:125-135, 2008). Compounds as described herein may be used to prevent or treat multiple sclerosis as well as other neurologic disorders associated with central nervous system inflammation, or maladaptive injury repair processes mediated by BMP signals.

Treatment of dementias—Inhibitors of BMP signaling can promote neurogenesis in mouse neural precursor cells (Koike et al. J. Biol. Chem. 282:15843-15850, 2007). Compounds as described herein can be used to augment neurogenesis in a variety of neurologic disorders associated with accelerated loss of neurons including cerebrovascular accidents and Alzheimer's Disease, as well as other dementias.

Altering memory and learning—BMP signaling has an important role in the development and maintenance of neurons involved in memory and cognitive behavior. For example, mice deficient in the BMP antagonist, chordin, have enhanced spatial learning but less exploratory activity in a novel environment (Sun et al. J. Neurosci. 27:7740-7750, 2007). Compounds as described herein can be used to alter or prevent memory or learning, for example, inducing amnesia for anesthesia or in other situations likely to cause distress, or to prevent Post-Traumatic Stress Disorder.

L. Treatment of Atherosclerosis

Abundant evidence suggests that BMP ligands are pro-inflammatory and pro-atherogenic in the blood vessel wall (Chang et al. Circulation 116:1258-1266, 2007). Knocking-down expression of BMP4 decreased inflammatory signals, whereas knocking-down BMP antagonists (eg follistatin or noggin) increased inflammatory signals. Compounds as described herein can be used to reduce vascular inflammation associated with atherosclerosis, automimmune disease, and other vasculitides. By decreasing atherosclerosis, it would be anticipated that compounds as described herein would decrease the incidence and/or severity of acute coronary syndromes (angina pectoris and heart attack), transient ischemic attacks, stroke, peripheral vascular disease, and other vascular ischemic events. Moreover, in so far as atherosclerosis contributes to the pathogenesis of aneurysm formation, compounds as described herein can be used to slow the progression of aneurysm formation decreasing the frequency of aneurismal rupture and the requirement for surgery.

As BMPs and many of the BMP-induced gene products that affect matrix remodeling are overexpressed in early atherosclerotic lesions, BMP signals may promote atherosclerotic plaque formation and progression (Bostrom et al. J Clin Invest. 91: 1800-1809. 1993; Dhore et al. Arterioscler Thromb Vasc Biol. 21: 1998-2003. 2001). BMP signaling activity in the atheromatous plaque may thus represent a form of maladaptive injury-repair, or may contribute to inflammation. Over time, BMP signals may also induce resident or nascent vascular cell populations to differentiate into osteoblast-like cells, leading to intimal and medial calcification of vessels (Hruska et al. Circ Res. 97: 105-112. 2005). Calcific vascular disease, or arteriosclerosis, is associated with decreased vascular distensibility, and increased risk of cardiovascular events and mortality, and is particularly problematic when associated with underlying atherosclerotic disease (Bostrom et al. Crit Rev Eukaryot Gene Expr. 10: 151-158. 2000). Both atherosclerotic and calcific lesions may be amenable to regression, however, if signals which contribute to their progression can be intercepted (Sano et al. Circulation. 103: 2955-2960. 2001). In certain aspects, compound 13 or another inhibitor of BMP type I receptor activity may be used to limit the progression of atheromatous plaques and vascular calcification in vivo.

M. Treatment of Hypercholesterolemia or Hyperlipoproteinemia

Treatment with small molecule or recombinant BMP inhibitors reduces vascular inflammation (via macrophage accumulation and cathepsin activity), atheroma formation, and vascular calcification in mice deficient in low-density lipoprotein receptor (LDLR^{-/-}).

Without wishing to be bound by theory, as potential explanations for impact on vascular inflammation, oxidized LDL (oxLDL) has been found to increase BMP2 expression and induce the production of reactive oxygen species (ROS) in human aortic endothelial cells. ROS production induced by oxLDL appears to require BMP signaling, based on inhibition by small molecule or recombinant BMP inhibitors. Treatment with small molecule BMP inhibitors reduces plasma low-density lipoprotein levels without inhibiting HMG-CoA reductase activity, suggesting a role of BMP signaling in the regulation of LDL cholesterol biosynthesis. Small molecule BMP inhibitors have also been found to inhibit hepatosteatosis seen in LDLR-deficient mice fed a high-fat diet. Small molecule or recombinant BMP inhibitors inhibit the synthesis of ApoB-100 in hepatoma cells in vitro. These findings implicate BMP signaling in vascular calcification and atherogenesis and provide at least two novel mechanisms by which BMP signaling may contribute to the pathogenesis of atherosclerosis. These studies highlight the BMP signaling pathway as a therapeutic target in the treatment of atherosclerosis while identifying several novel functions of BMP signaling in the regulation of vascular oxidative stress, inflammation and lipid metabolism.

In certain embodiments, BMP inhibitors as described herein may be used for the reduction of circulating levels of ApoB-100 in patients. In certain embodiments, BMP inhibitors as described herein may be used for the reduction of circulating levels of LDL in patients. Accordingly, BMP inhibitors as described herein may be used for the treatment of hypercholesterolemia, hyperlipidemia, or hyperlipoproteinemia, including congenital or acquired hypercholesterolemia, hyperlipidemia, or hyperlipoproteinemia.

In certain embodiments, the congenital hypercholesterolemia, hyperlipidemia, or hyperlipoproteinemia is autosomal dominant hypercholesterolemia (ADH), familial hypercholesterolemia (FH), polygenic hypercholesterolemia, familial combined hyperlipidemia (FCHL), hyperapobetalipoproteinemia, or small dense LDL syndrome (LDL phenotype B).

In certain embodiments, the acquired hypercholesterolemia, hyperlipidemia, or hyperlipoproteinemia is associated with diabetes mellitus, hyperlipidemic diet and/or sedentary lifestyle, obesity, metabolic syndrome, intrinsic or secondary liver disease, primary biliary cirrhosis or other bile stasis disorders, alcoholism, pancreatitis, nephrotic syndrome, endstage renal disease, hypothyroidism, iatrogenesis due to administration of thiazides, beta-blockers, retinoids, highly active antiretroviral agents, estrogen, progestins, or glucocorticoids.

In certain embodiments, BMP inhibitors as described herein may be used for the treatment of diseases, disorders, or syndromes associated with defects in lipid absorption or metabolism, such as sitosterolemia, cerebrotendinous xanthomatosis, or familial hypobetalipoproteinemia.

In certain embodiments, BMP inhibitors as described herein may be used for the treatment of diseases, disorders, or syndromes caused by hyperlipidemia, such as coronary artery disease and its manifestations (e.g., myocardial infarction; angina pectoris; acute coronary artery syndromes, such as unstable angina pectoris; cardiac dysfunction, such as congestive heart failure, caused by myocardial infarction; or cardiac arrhythmia associated with myocardial ischemia/infarction), stroke due to occlusion of arteries supplying portions of the brain, cerebral hemorrhage, peripheral arterial disease (e.g., mesenteric ischemia; renal artery stenosis; limb ischemia and claudication; subclavian steal syndrome; abdominal aortic aneurysm; thoracic aortic aneurysm, pseudoaneurysm, intramural hematoma; or penetrating aortic ulcer, aortic dissection, aortic stenosis, vascular calcification, xanthoma, such as xanthoma affecting tendons or scleral and cutaneous xanthomas, xanthelasma, or hepatosteatosis. In certain embodiments, BMP inhibitors as described herein may be used for the treatment of the foregoing diseases, disorders, or syndromes regardless of circulating lipid levels, such as in individuals exhibiting normal circulating lipid levels or metabolism.

In certain embodiments, BMP inhibitors as described herein may be used for the reduction of secondary cardiovascular events arising from coronary, cerebral, or peripheral vascular disease. In certain such embodiments, BMP inhibitors as described herein may be used to treat individuals regardless of lipid levels, such as used in the treatment of individuals exhibiting normal circulating cholesterol and lipid levels. In certain such embodiments, BMP inhibitors as described herein are administered conjointly with a HMG-CoA reductase inhibitor.

In certain embodiments, BMP inhibitors as described herein may be used for the prevention of cardiovascular disease, such as in individuals with elevated markers of cardiovascular risk (e.g., C-reactive protein) or, for example, an elevated Framingham Risk Score. In certain such embodiments, BMP inhibitors as described herein may be used to prevent cardiovascular disease in individuals exhibiting normal circulating cholesterol and lipid levels.

In certain embodiments wherein one or more BMP inhibitors as described herein are used in the treatment or prevention of the foregoing diseases, disorders, or syndromes, the patient being treated is not diagnosed with and/or is not suffering from one or more of the following conditions: vascular inflammation associated with atherosclerosis, automimmune disease, and other vasculitides; atherosclerotic disease, atheromatous plaques, and/or vascular calcification; an aneurysm and/or aneurysm formation; acute coronary syndromes (angina pectoris and heart attack), transient ischemic attacks, stroke, peripheral vascular disease, or other vascular ischemic events.

In other embodiments wherein one or more BMP inhibitors as described herein are used in the treatment or prevention of the foregoing diseases, disorders, or syndromes (e.g., for the reduction of circulating levels of ApoB-100 and/or LDL in patients; for the treatment of hypercholesterolemia, hyperlipidemia, or hyperlipoproteinemia, including congenital or acquired hypercholesterolemia, hyperlipidemia, or hyperlipoproteinemia; for the treatment of diseases, disorders, or syndromes associated with defects in lipid absorption or metabolism; for the treatment of diseases, disorders, or syndromes caused by hyperlipidemia; for the reduction of secondary cardiovascular events arising from coronary, cerebral, or peripheral vascular disease; or for the reduction of secondary cardiovascular events arising from coronary, cerebral, or peripheral vascular disease), the patient being treated is also diagnosed with and/or is also suffering from one or more of the following conditions: vascular inflammation associated with atherosclerosis, automimmune disease, and other vasculitides; atherosclerotic disease, atheromatous plaques, and/or vascular calcification; an aneurysm and/or aneurysm formation; acute coronary syndromes (angina pectoris and heart attack), transient ischemic attacks, stroke, peripheral vascular disease, or other vascular ischemic events.

N. Propagation, Engraftment and Differentiation of Progenitor Cells Including Embryonic and Adult Stem Cells In Vitro and In Vivo

BMP signals are crucial for regulating the differentiation and regeneration of precursor and stem cell populations, in some contexts and tissues preventing (while in other contexts directing) differentiation towards a lineage. Compounds as described herein can be used to (i) maintain a pluripotential state in stem cell or multipotent cell populations in vivo or in vitro; (ii) expand stem cell or multipotent cell populations in vivo or in vitro; (iii) direct differentiation of stem cell or multipotent cell populations in vivo or in vitro; (iv) manipulate or direct the differentiation of stem cell or multipotent cell populations in vivo or in vitro, either alone or in combination or in sequence with other treatments; and (v) modulate the de-differentiation of differentiated cell populations into multipotent or progenitor populations.

Numerous stem cell and precursor lineages require BMP signals in order to determine whether they will expand, differentiate towards specific tissue lineages, home in and integrate with particular tissue types, or undergo programmed cell death. Frequently BMP signals interact with signals provided by growth factors (bFGF, PDGF, VEGF, HBEGF, P1GF, and others), Sonic Hedgehog (SHH), notch, and Wnt signaling pathways to effect these changes (Okita et al. Curr. Stem Cell Res. Ther. 1:103-111, 2006). Compounds as described herein can be used to direct the differentiation of stem cells (e.g., embryonic stem cells) or tissue progenitor cells towards specific lineages for therapeutic application (Park et al. Development 131:2749-2762, 2004; Pashmforoush et al. Cell 117:373-386, 2004). Alternatively for certain cell populations, BMP inhibitors as described herein may be effective in preventing differentiation and promoting expansion, in order to produce sufficient numbers of cells to be effective for a clinical application. The exact combination of BMP antagonist and growth factor or signaling molecule may be highly specific to each cell and tissue type.

For example, certain embryonic stem cell lines require co-culture with leukemia inhibitory factor (LIF) to inhibit differentiation and maintain the pluripotency of certain cultured embryonic stem cell lines (Okita et al. Curr. Stem Cell Res. Ther. 1:103-111, 2006). Use of a BMP inhibitor as described herein may be used to maintain pluripotency in the absence of LIF. Other ES cell lines require coculture with a specific feeder cell layer in order to maintain pluripotency. Use of a BMP inhibitor as described herein, alone or in combination with other agents, may be effective in maintaining pluripotency when concerns of contamination with a feeder cell layer, or its DNA or protein components would complicate or prevent use of cells for human therapy.

In another example, in some circumstances antagonizing BMP signals with a protein such as noggin shortly before cessation of LIF in culture is able to induce differentiation into a cardiomyocyte lineage (Yuasa et al. Nat. Biotechnol. 23:607-611, 2005). Use of a pharmacologic BMP antagonist as described herein may achieve similar if not more potent effects. Such differentiated cells could be introduced into diseased myocardium therapeutically. Alternatively, such treatment may actually be more effective on engrafted precursor cells which have already homed in to diseased myocardium. Systemic therapy with a protein antagonist of BMP such as noggin would be prohibitively expensive and entail complicated dosing. Delivery of a BMP antagonist as described herein, systemically or locally, could bias the differentiation of such precursor cells into functioning cardiomyocytes in situ.

O. Application of Compounds with Varying Degrees of Selectivity: Compounds which Inhibit BMP Signaling Via Particular BMP Type I Receptors, or Compounds which Also Affect Signaling Via TGF-β, Activin, AMP Kinase, or VEGF Receptors

ALK-specific antagonists—Dorsomorphin inhibits the activity of the BMP type I receptors, ALK2, ALK3, and ALK6. Dorsomorphin inhibits ALK2 and ALK3 to a greater extent than it does ALK6 (Yu et al. Nat. Chem. Biol. 4:33-41, 2008). Several of the compounds described herein will have relative greater selectivity for particular BMP type I receptors. The pathogenesis of certain diseases might be attributed to the dysfunctional signaling of one particular receptor. For example, fibrodysplasia ossificans progressiva is a disease caused by aberrant (constitutively active) ALK2 function (Yu et al. Nat. Chem. Biol. 4:33-41, 2008). In such instances, compounds as described herein which specifically antagonize the function a subset of the BMP type I receptors may have the advantage of reduced toxicity or side effects, or greater effectiveness, or both.

Some compounds as described herein may have a high degree of selectivity for BMP vs. TGF-β, Activin, AMP kinase, and VEGF receptor signaling. Other compounds may be less specific and may target other pathways in addition to BMP signaling. In the treatment of tumors, for example, agents which inhibit BMP signaling as well as one or more of the above pathways can have beneficial effects (e.g. decrease tumor size), when molecular phenotyping of specific patients' tumors reveals dysregulation of multiple pathways.

P. Applications of Compounds in Species Other than Human

Compounds as described herein can be used to treat subjects (e.g., humans, domestic pets, livestock, or other animals) by use of dosages and administration regimens that are determined to be appropriate by those of skill in the art, and these parameters may vary depending on, for example, the type and extent of the disorder treated, the overall health status of the subject, the therapeutic index of the compound, and the route of administration. Standard clinical trials can be used to optimize the dose and dosing frequency for any particular pharmaceutical composition of the invention. Exemplary routes of administration that can be used include oral, parenteral, intravenous, intra-arterial, subcutaneous, intramuscular, topical, intracranial, intraorbital, ophthalmic, intraventricular, intracapsular, intraspinal, intracisternal, intraperitoneal, intranasal, aerosol, or administration by suppository. Methods for making formulations that can be used in the invention are well known in the art and can be found, for example, in Remington: The Science and Practice of Pharmacy (20th edition, Ed., A. R. Gennaro), Lippincott Williams & Wilkins, 2000.

Q. Combination Therapies

In certain instances BMP antagonists as described herein may be used in combination with other current or future drug therapies, because the effects of inhibiting BMP alone may be less optimal by itself, and/or may be synergistic or more highly effective in combination with therapies acting on distinct pathways which interact functionally with BMP signaling, or on the BMP pathway itself. In certain instances, conjoint administration of a BMP antagonist as described herein with an additional drug therapy reduces the dose of the additional drug therapy such that it is less than the amount that achieves a therapeutic effect when used in a monotherapy (e.g., in the absence of a BMP antagonist as described herein). Some examples of combination therapies could include the following.

In certain embodiments, BMP antagonists as described herein may be administered conjointly with other antihyperlipidemic agents or antilipidemic agents including, but not limited to, HMG-CoA reductase inhibitors (e.g., atorvastatin, cerivastatin, fluvastatin, lovastatin, mevastatin, pitavastain, pravastatin, rosuvastatin, or simvastatin), fibrates (e.g., bezafibrate, ciprofibrate, clofibrate, gemfibrozil, or fenofibrate), ezetimibe, niacin, cholesteryl ester transfer protein (CETP) inhibitors (e.g., torcetrapib, anacetrapib, or dalcetrapib), cholestyramine, colestipol, probucol, dextrothyroxine, bile acid sequestrants, or combinations of the above.

In certain embodiments, BMP antagonists as described herein may be administered conjointly with a treatment for diabetes including, but not limited to, sulfonyl ureas (e.g., chlorpropamide, tolbutamide, glyburide, glipizide, or glimepiride), medications that decrease the amount of glucose produced by the liver (e.g., metformin), meglitinides (e.g., repaglinide or nateglinide), medications that decrease the absorption of carbohydrates from the intestine (e.g., alpha glucosidase inhibitors such as acarbose), medications that effect glycemic control (e.g., pramlintide or exenatide), DPP-IV inhibitors (e.g., sitagliptin), insulin treatment, thiazolidinones (e.g., troglitazone, ciglitazone, pioglitazone, or rosiglitazone), oxadiazolidinediones, alpha-glucosidase inhibitors (e.g., miglitol or acarbose), agents acting on the ATP-dependent postassium channel of the beta cells (e.g., tolbutamide, glibenclamide, glipizide, glicazide, or repaglinide), nateglinide, glucagon antagonists, inhibitors of hepatic enzymes involved in stimulation of gluconeogenesis and/or glycogenolysis, or combinations of the above.

In certain embodiments, BMP antagonists as described herein may be administered conjointly with a treatment for obesity including, but not limited to, orlistat, sibutramine, phendimetrazine, phentermine, diethylpropion, benzphetamine, mazindol, dextroamphetamine, rimonabant, cetilistat, GT 389-255, APD356, pramlintide/AC137, PYY3-36, AC 162352/PYY3-36, oxyntomodulin, TM 30338, AOD 9604, oleoyl-estrone, bromocriptine, ephedrine, leptin, pseudoephedrine, or pharmaceutically acceptable salts thereof, or combinations of the above.

In certain embodiments, BMP antagonists as described herein may be administered conjointly with an antihypertensive agent including, but not limited to, beta-blockers (e.g., alprenolol, atenolol, timolol, pindolol propranolol and metoprolol), ACE (angiotensin converting enzyme) inhibitors (e.g., benazepril, captopril, enalapril, fosinopril, lisinopril, quinapril and ramipril), calcium channel blockers (e.g., nifedipine, felodipine, nicardipine, isradipine, nimodipine, diltiazem and verapamil), and alpha-blockers (e.g., doxazosin, urapidil, prazosin and terazosin), or combinations of the above.

Tyrosine kinase receptor inhibitors, such as SU-5416, and BMP antagonists as described herein may have synergistic effects at inhibiting angiogenesis, particularly for anti-angiogenic therapy against tumors. BMP signals (BMP-4) are thought to be critical for the commitment of stem or precursor cells to a hematopoietic/endothelial common progenitor, and may promote the proliferation, survival, and migration of mature endothelial cells necessary for angiogenesis (Park et al. Development 131:2749-2762, 2004). Thus antagonism of BMP signals using compounds as described herein may provide additional inhibition of angiogenesis at the level of endothelial precursors and cells. Similarly, co-treatment with BMP antagonists as described herein and other tyrosine kinase receptor inhibitors such as imatinib (Gleevec) could be used to inhibit vascular remodeling and angiogenesis of certain tumors.

The combination of a sonic hedgehog agonist and a BMP antagonist as described herein may be particularly useful for promoting hair growth, as SHH activity is known to stimulate the transition of follicles out of telogen (resting) phase (Paladini et al. J. Invest. Dermatol. 125:638-646, 2005), while inhibiting the BMP pathway shortens the telogen phase (Plikus et al. Nature 451:340-344, 2008). The use of both would be expected to cause relatively increased time in the anagen or growth phase.

Combined use of Notch modulators (e.g., gamma-secretase inhibitors) and BMP antagonists as described herein may be more effective than either agent alone in applications designed to inhibit vascular remodeling or bone differentiation, because increasing evidence suggests both pathways function cooperatively to effect cell differentiation, and vascular cell migration (Kluppel et al. Bioessays 27:115-118, 2005). These therapies may be synergistic in the treatment of tumors in which one or both pathways is deranged (Katoh, Stem Cell Rev. 3:30-38, 2007).

Combined use of an Indian Hedgehog (IHH) antagonist and a BMP antagonist as described herein may inhibit pathologic bone formation. IHH is responsible for the commitment of bone precursors to chondrocyte or cartilage forming cells. Endochondral bone formation involves coordinated activity of both chondrogenesis (promoted by BMP signals and IHH signals) and their subsequent calcification by mineralization programs initiated by BMP signals (Seki et al. J. Biol. Chem. 279:18544-18549, 2004; Minina et al. Development 128:4523-4534, 2001). Coadministration of an IHH antagonist with a BMP antagonist as described herein, therefore, may be more effective in inhibiting pathological bone growth due to hyperactive BMP signaling (such as in FOP), or in any of the inflammatory or traumatic disorders of pathologic bone formation described above.

Strong experimental evidence exists for an effect of both Smo antagonism and BMP antagonism for treating glioblastoma. Compounds as described herein may be used in combination with Smo antagonists to treat glioblastoma.

R. Inhibition of BMP Signaling in Insects

Some of the compounds as described herein may have activity against, and perhaps even selectivity for the BMP receptors of arthropods versus those of chordates. Inhibiting BMP signaling in arthropod larvae or eggs is likely to cause severe developmental abnormalities and perhaps compromise their ability to reproduce, e.g., via the same dorsalization that is observed in zebrafish and drosophila when this pathway is inhibited. If BMP antagonists as described herein have very strong selectivity for arthropod BMP receptors versus those of humans, they may be used as insecticides or pest control agents that are demonstrably less toxic or more environmentally sound than current strategies.

In addition to being administered to patients in therapeutic methods, compounds as described herein can also be used to treat cells and tissues, as well as structural materials to be implanted into patients (see above), ex vivo. For example, the compounds can be used to treat explanted tissues that may be used, for example, in transplantation.

The invention now being generally described, it will be more readily understood by reference to the following examples which are included merely for purposes of illustration of certain aspects and embodiments of the present invention, and are not intended to limit the invention.

### EXEMPLIFICATION

The synthesis and in vitro and in vivo evaluation of certain BMP inhibitors disclosed herein is set forth in WO 2009/114180, which is herein incorporated by reference in its entirety.

### Example 1

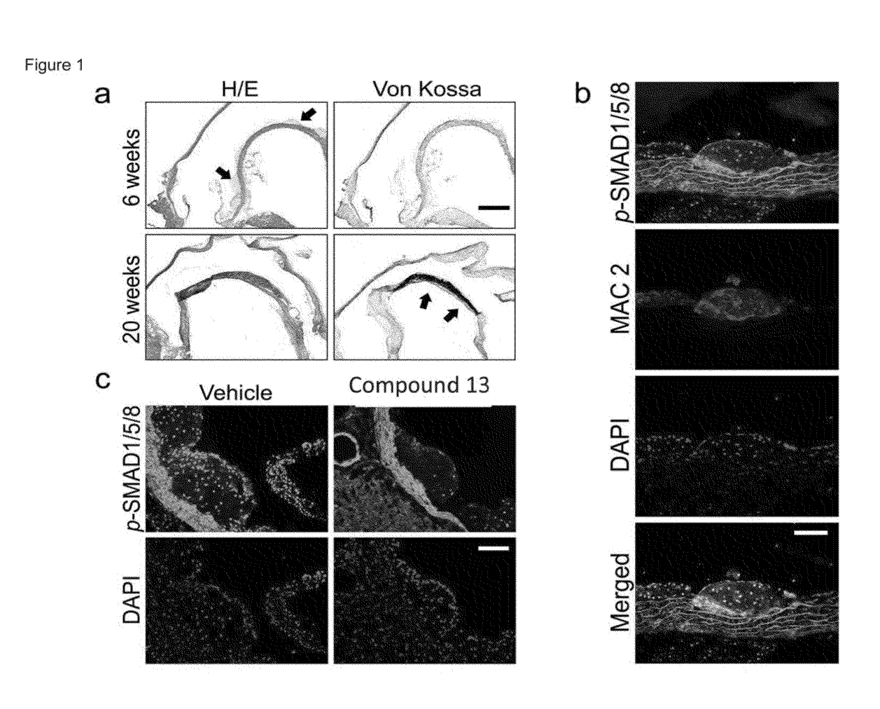

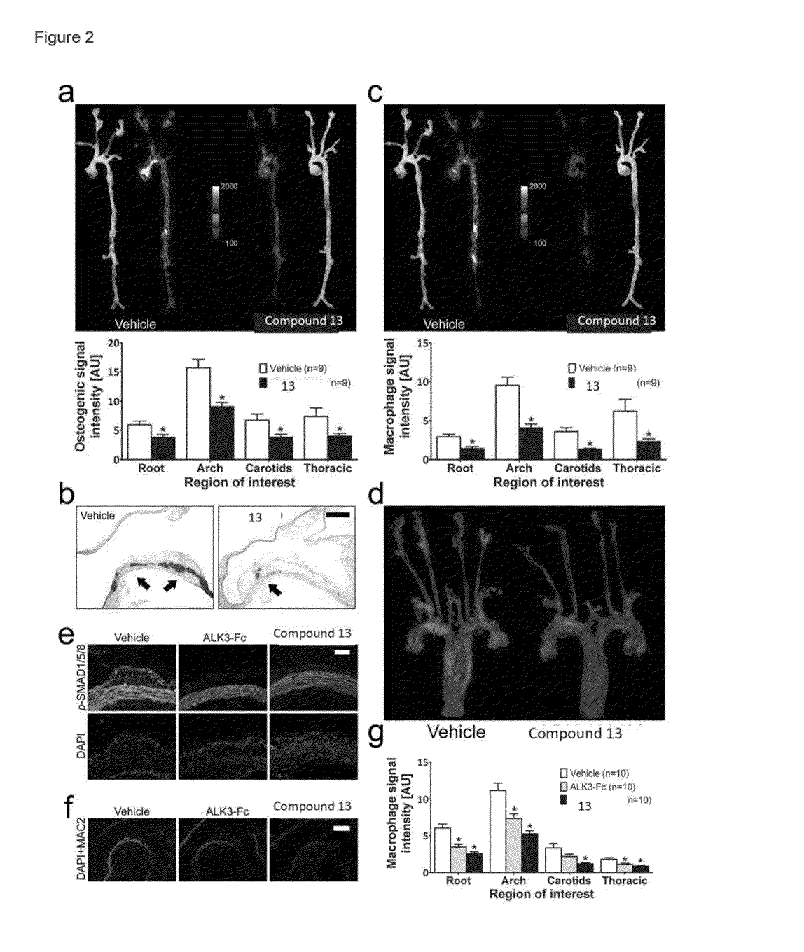

To elucidate the role of BMP signaling in vascular calcification and atherogenesis, BMP signaling was inhibited in vivo using small molecule and recombinant protein approaches. Mice deficient in the low-density lipoprotein receptor (LDLR^{-/-}) fed a high fat diet (HFD) were studied. (Ishibashi, S., Goldstein, J. L., Brown, M. S., Herz, J. & Burns, D. K. Massive xanthomatosis and atherosclerosis in cholesterol-fed low density lipoprotein receptor-negative mice. J Clin Invest 93, 1885-1893 (1994).) These mice developed atheroma within 4-6 weeks followed by intimal and medial calcification by 16-20 weeks (FIG. 1a). At 20 weeks, there was extensive vascular calcification (as reflected by fluorescence labeled bisphosphonate uptake, Alizarin red staining and Von Kossa staining), inflammation (as reflected by cathepsin mediated cleavage of a near infrared imaging probe) and abundant lipid accumulation (as reflected by Oil Red O) in the aorta and large-vessel branches. (FIG. 2a-d)

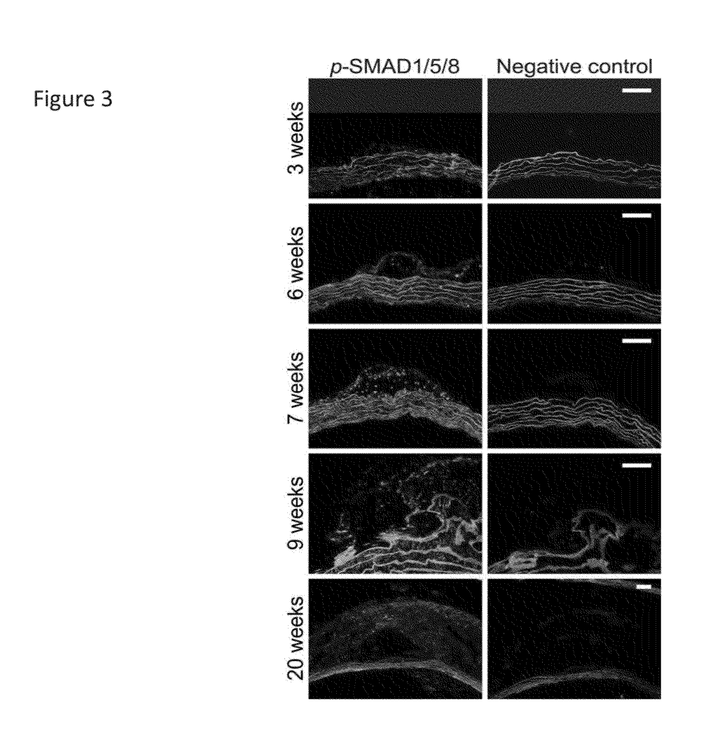

BMP signaling activity in the nascent lesions of LDLR^{-/-} mice was characterized. Phosphorylated BMP-responsive SMADs (p-SMAD1/5/8), effectors of the BMP signaling pathway that are retained in the nuclei of activated cells, were detected by immunofluorescence in endothelial cells, macrophages, and the media of atheromatous lesions (FIG. 1b), particularly in the lesser curvature of the aorta, beginning three weeks on HFD. Activation of BMP signaling persisted for at least 20 weeks and was abundant near calcific lesions (FIG. 3). To confirm that the activation of SMAD1/5/8 observed in the vessels of LDLR^{-/-} mice was attributable to BMP signaling, a BMP type I receptor inhibitor, compound 13, was administered (2.5 mg/kg ip daily for five days) to LDLR^{-/-} mice that had received HFD for 6 weeks. BMP type I receptor inhibition markedly diminished the detection of p-SMAD1/5/8 within atherosclerotic lesions (FIG. 1c).

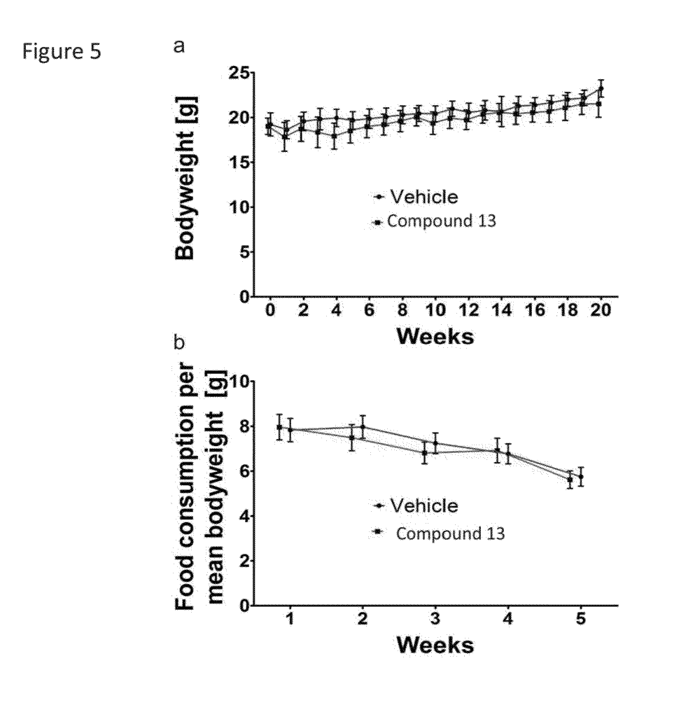

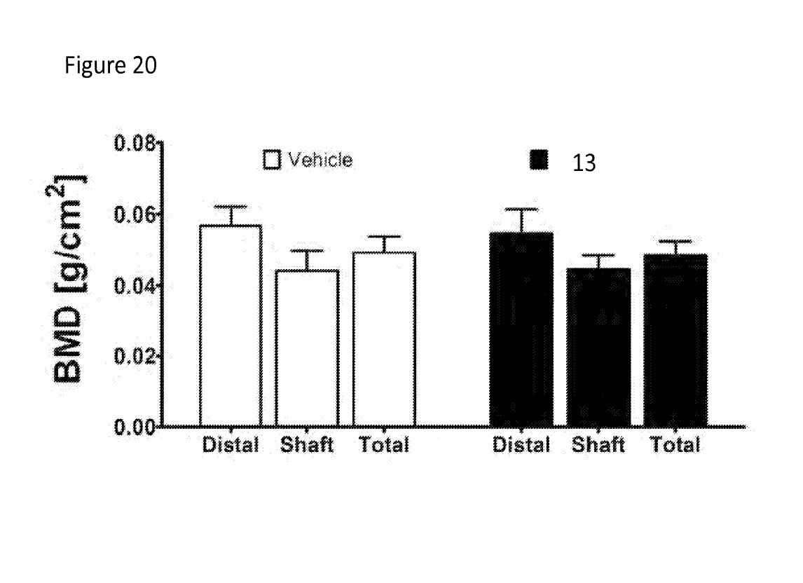

Whether the activation of BMP signaling observed following HFD administration in LDLR^{-/-} mice was required for vascular calcification was then determined. Administration of compound 13 (2.5 mg/kg ip daily) to LDLR^{-/-} mice while receiving HFD for 20 weeks reduced vascular calcification throughout the aortae, based on reduced uptake of fluor-labeled bisphosphonate and diminished Alizarin red staining (FIG. 2a and b). The reduction of vascular calcification by treatment with compound 13 was accompanied by a similar marked reduction in vascular inflammation (FIG. 2c) and lipid accumulation (FIG. 2d). The impact of compound 13 on vascular inflammation and calcification was not associated with a reduction in body weight or food intake (FIG. 5 a, b). These results suggested that BMP inhibition might ameliorate atherogenesis and associated vascular inflammation in addition to calcification. Bone mineral density also did not differ between LDLR^{-/-} mice treated with vehicle or compound 13 (FIG. 20). Bone mineral density was measured in femurs from sacrificed LDLR^{-/-} mice fed a HFD fro 20 weeks while receiving daily injections of vehicle (n=8) or compound 13 (n=10, 2.5 mg/kg ip) using dual energy X-ray absorptiometry in the distal femur (Distal, FIG. 20), the femur shaft (Shaft, FIG. 20) or in the whole bone (Total, FIG. 20, mean±SEM).

To confirm that the effects of compound 13 on vascular inflammation and atheroma were due to inhibition of BMP signaling rather than an off-target effect, the impact of a recombinant BMP inhibitor (ALK3-Fc) during atheroma formation was tested in this model. Administration of ALK3-Fc (2 mg/kg ip every other day) to LDLR^{-/-} mice while receiving HFD for six weeks reduced the detection of p-SMAD1/5/8 (FIG. 2e), macrophage burden (FIG. 2f), and cathepsin activity throughout the aorta (FIG. 2g), as did treatment with compound 13 over the same period. The efficacy of two distinct BMP inhibition strategies provides compelling evidence that BMP signaling contributes to atherogenesis and associated vascular inflammation.

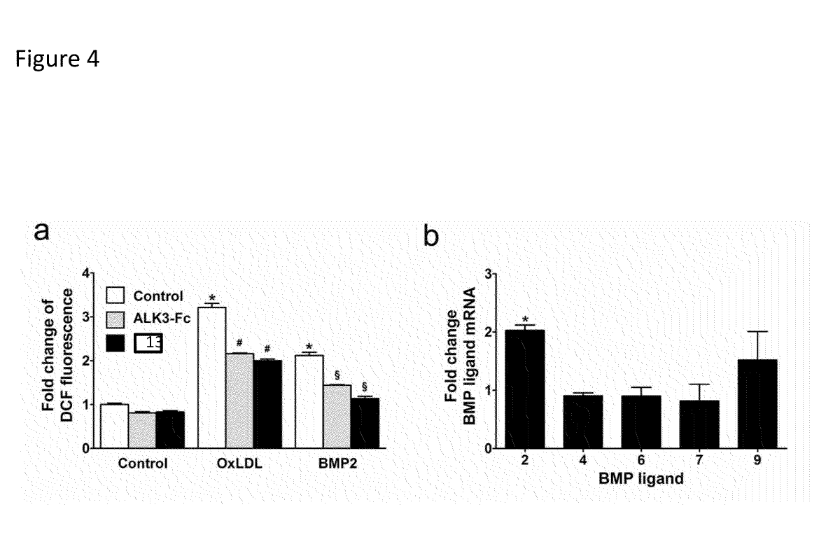

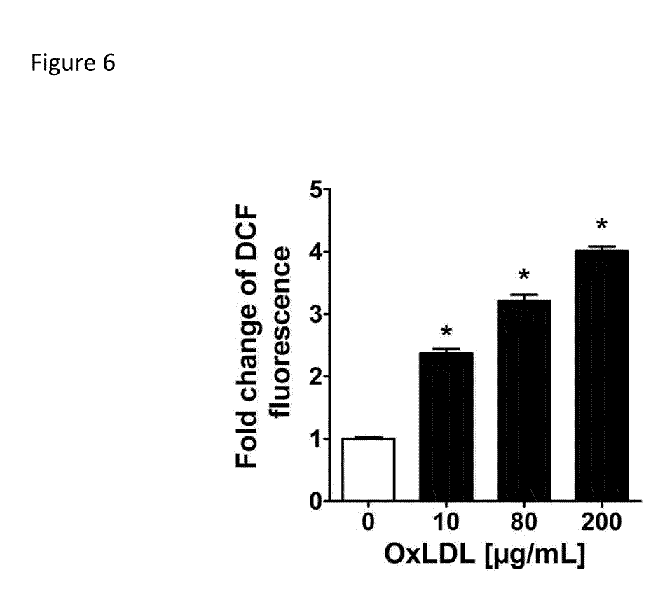

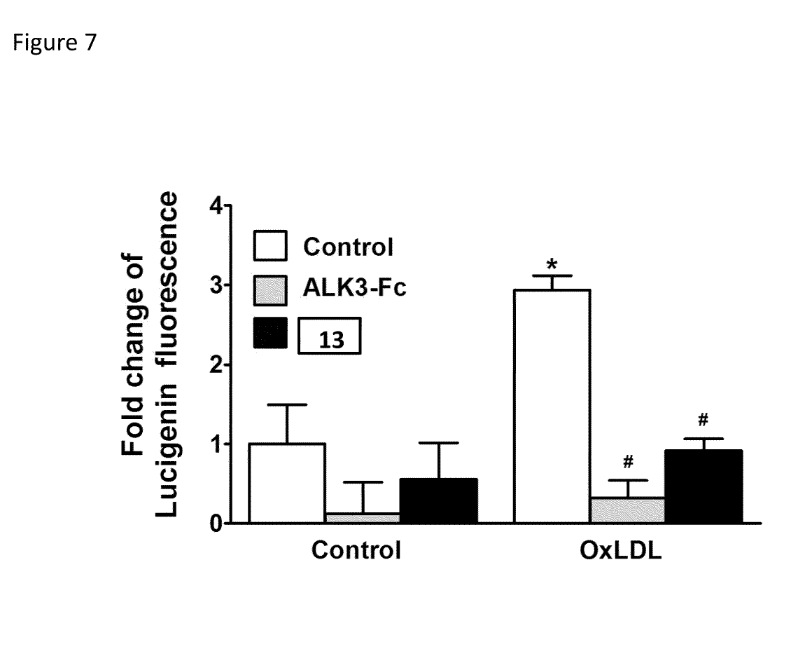

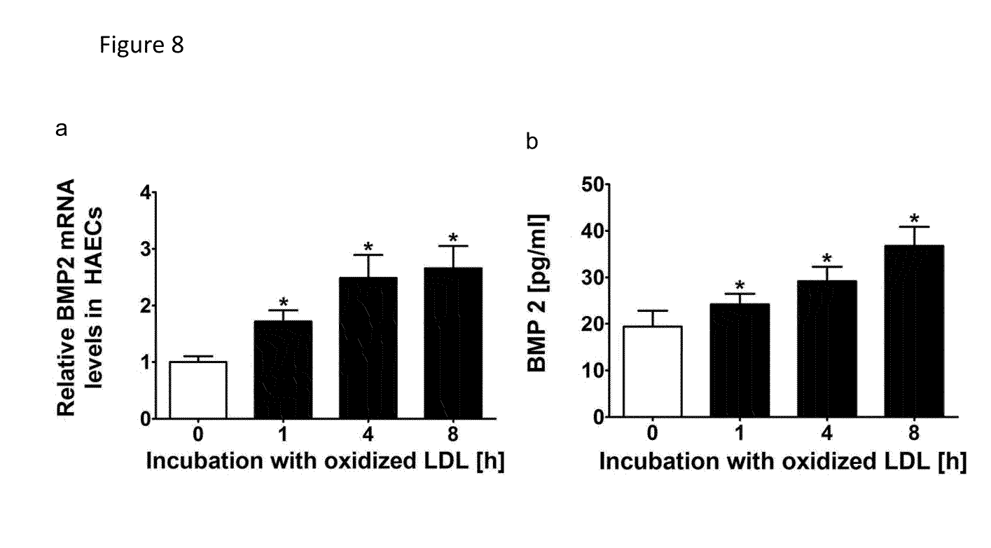

The activation of endothelial cells by oxidized LDL (oxLDL) reflected by an increase in reactive oxygen species (ROS) has been implicated in the pathogenesis of atherosclerosis and vascular calcification. (Levitan, I., Volkov, S. & Subbaiah, P. V. Oxidized LDL: diversity, patterns of recognition, and pathophysiology. Antioxid Redox Signal 13, 39-75 (2010).) To gain insight into how BMP inhibition might impact vascular inflammation or oxidative stress, ROS production in oxLDL-exposed human aortic endothelial cells (HAECs) pre-treated with either compound 13, ALK3-Fc, or vehicle was tested. It was observed that oxLDL increased ROS production in untreated HAECs (FIG. 6), and that both compound 13 and ALK3-Fc could inhibit oxLDL-induced ROS production (FIG. 4a and FIG. 7). To identify the BMP ligand responsible for the production of ROS, levels of mRNA encoding BMP ligands in HAECs exposed to oxLDL for 8 hours were measured. Exposure of HAECs to oxLDL did not alter BMP4, BMP6, BMP7, or BMP9 mRNA levels (FIG. 4b), but increased BMP2 mRNA and protein levels (FIGS. 8a and b). Moreover, incubation of HAECs with BMP2 increased ROS generation in a compound 13- and ALK3-Fc-sensitive manner (FIG. 4a). These results demonstrate that oxLDL induces endothelial cells to generate ROS, in part, via a BMP-dependent mechanism, likely mediated by BMP2, and suggest that BMP-mediated activation of endothelial cells may play an important role in the pathogenesis of atherosclerosis and vascular calcification.

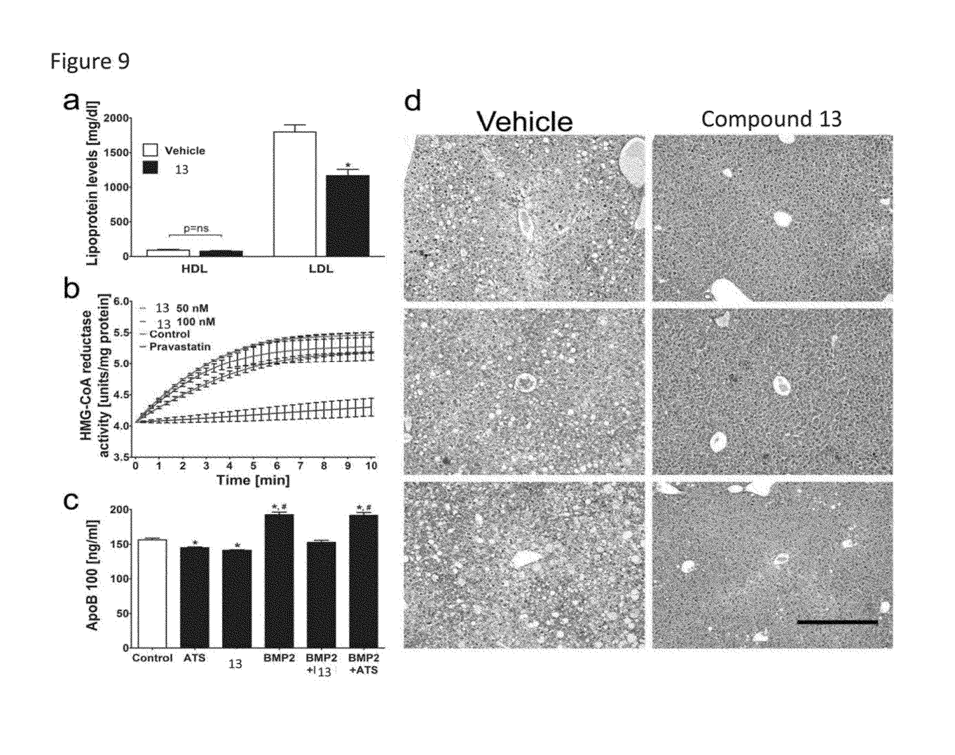

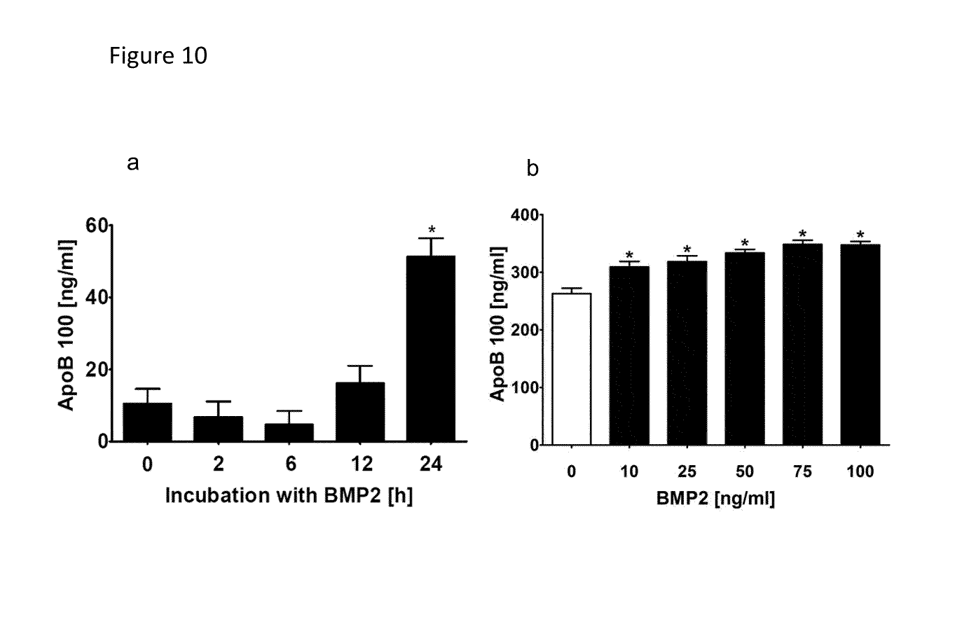

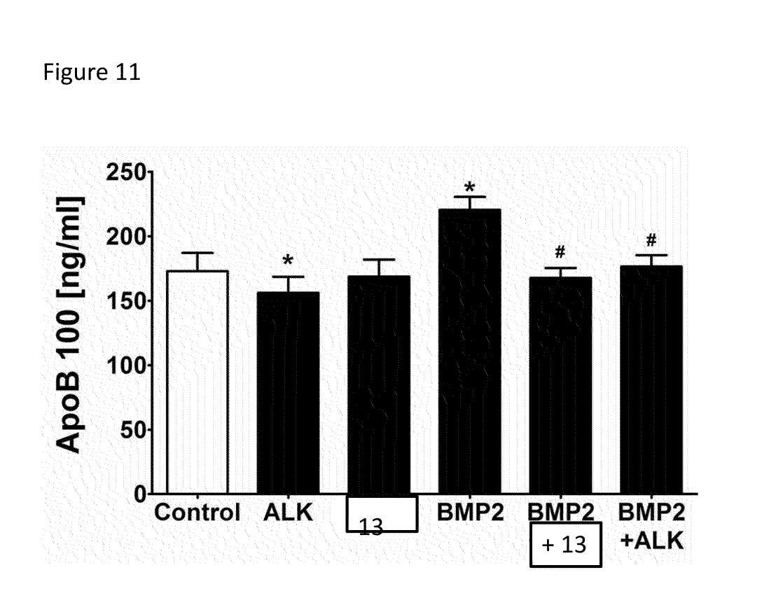

Serum lipoprotein levels are known to be an important risk factor for atherosclerosis, and total cholesterol and LDL levels are markedly elevated in LDLR^{-/-} mice (FIG. 9a and Table 1). It was observed that compound 13 treatment reduced total cholesterol levels and LDL levels, but not HDL levels, in LDLR^{-/-} mice fed a HFD for 20 weeks (FIG. 9a and Table 1). Moreover, compound 13 treatment reduced total serum cholesterol in wild-type animals on a HFD (Table 2). The ability of compound 13 to reduce LDL levels did not appear to be mediated by a direct effect on HMG-CoA reductase activity, based on the lack of impact of compound 13 upon enzyme activity in vitro (FIG. 9b). To investigate the potential role of BMP signaling in the regulation of LDL synthesis, apolipoprotein B100 (ApoB-100) production by HepG2 cells in the presence or absence of BMP2 with and without BMP inhibitors was measured. It was observed that incubation of HepG2 with BMP2 for 24 hours increased ApoB-100 production in a dose-dependent manner (FIG. 9c and FIGS. 10a and b). Incubation with compound 13 (FIG. 9c) and ALK3-Fc (FIG. 11) inhibited ApoB-100 production by HepG2 cells in the absence of BMP2 and prevented the BMP2-mediated induction of ApoB-100 secretion. In contrast, the HMG-CoA reductase inhibitor, atorvastatin, reduced ApoB-100 production in the absence of BMP2, but did not prevent the induction of ApoB-100 synthesis by BMP2. Taken together, these findings suggest a novel role for BMP signaling in the regulation of lipoprotein biosynthesis.

TABLE 1
Blood biochemical analysis in LDLR^{-/-} mice 20 weeks
on high fat diet. Female LDLR^{-/-} were started on
a high fat diet at eight weeks of age for 20 weeks and received
daily injections of either Vehicle or compound 13 (2.5 mg/kg
ip). Student's t-test. Data presented as mean ± SEM.

| | Vehicle | Compound 13 | p= |
|---|---|---|---|
| Cholesterol [mg/dl] | 1957 ± 159 | 1401 ± 87 | 0.01 |
| HDL [mg/dl] | 89.8 ± 30.2 | 73.8 ± 26.5 | 0.268 |
| LDL [mg/dl] | 1797.2 ± 306.6 | 1166.3 ± 257.3 | 0.001 |
| Triglycerides [mg/dl] | 125 ± 23 | 135 ± 16 | 0.73 |
| Hemoglobin [g/dl] | 11.6 ± 1.0 | 12.9 ± 0.8 | 0.34 |
| Blood urea nitrogen [mg/dl] | 25.1 ± 1 | 25 ± 2 | 0.84 |
| Glucose [mg/dl] | 216 ± 21 | 230 ± 19 | 0.65 |
| Alkaline phosphatase [IU/L] | 158 ± 15 | 84 ± 11 | 0.00 |
| Total protein [g/dl] | 4.9 ± 0.1 | 4.3 ± 0.4 | 0.12 |
| Alanine transaminase [IU/L] | 257 ± 44 | 130 ± 21 | 0.03 |
| Creatinine [mg/dl] | 0.5 ± 0.0 | 0.4 ± 0.0 | 0.61 |
| | n = 10 | n = 8 | |

TABLE 2
Blood biochemical analysis in WT mice fed a high fat diet
for 30 weeks. Female C57BL/6 were started on a high fat
diet at eight weeks of age for 30 weeks and received daily
injections of either Vehicle or compound 13 (2.5 mg/kg ip).
Student's t-test. Data presented as mean ± SEM.

| | Vehicle | Compound 13 | p= |
|---|---|---|---|
| Cholesterol [mg/dl] | 218 ± 5 | 183 ± 6 | 0.00 |
| Triglycerides [mg/dl] | 144 ± 26 | 82 ± 21 | 0.10 |
| Hemoglobin [g/dl] | 13.7 ± 0.9 | 13.5 ± 1.0 | 0.89 |
| Blood urea nitrogen [mg/dl] | 32 ± 4 | 25 ± 1 | 0.09 |
| Glucose [mg/dl] | 283 ± 16 | 311 ± 12 | 0.17 |
| Alkaline phosphatase [IU/L] | 177 ± 12 | 109 ± 8 | 0.00 |
| Total protein [g/dl] | 4.8 ± 0.1 | 4.7 ± 0.2 | 0.56 |
| Alanine transaminase [IU/L] | 275 ± 21 | 159 ± 19 | 0.00 |
| Creatinine [mg/dl] | 0.6 ± 0.0 | 0.5 ± 0.0 | 0.06 |
| | n = 8 | n = 9 | |

It is conceivable that the effect of compound 13 on atherogenesis is solely attributable to its ability to reduce lipoprotein levels. However, treatment of LDLR^{-/-} fed a HFD for six weeks with ALK3-Fc inhibited atherogenesis without altering LDL or total cholesterol levels (Table 3). These results suggest that BMP signaling has a role in atherogenesis above and beyond its impact on lipoprotein biosynthesis, e.g., in the vascular wall. It is unknown why compound 13 reduces lipoprotein levels but ALK3-Fc does not in LDLR^{-/-} mice. Possible explanations of why ALK3-Fc does not reduce lipoprotein levels in vivo include differences in bioavailability, spectrum of intercepted BMP ligands, and differences in pharmacokinetics or pharmacodynamics.

TABLE 3
Blood biochemical analysis in LDLR^{-/-} mice fed
a high fat diet for 6 weeks. Female LDLR^{-/-} were
started on a high fat diet at eight weeks of age for six
weeks and received daily injections of either Vehicle
or compound 13 (2.5 mg/kg ip), or ALK3-Fc (2 mg/kg ip)
every other day. Data presented as mean ± SEM.

| | Vehicle | ALK3-Fc | Compound 13 |
|---|---|---|---|
| Cholesterol | 1953 ± 102 | 2206 ± 139 | 1553 ± 77*^{,§} |
| [mg/dl] | | | |
| Triglycerides | 122 ± 10 | 108 ± 11 | 112 ± 15 |
| [mg/dl] | | | |
| Hemoglobin | 13.7 ± 1.3 | 14.2 ± 1.1 | 12.9 ± 1.5 |
| [g/dl] | | | |
| Blood urea nitrogen | 28 ± 1 | 25 ± 1 | 24 ± 2 |
| [mg/dl] | | | |
| Glucose | 253 ± 19 | 243 ± 16 | 259 ± 20 |
| [mg/dl] | | | |
| Alkaline phosphatase | 141 ± 14 | 207 ± 22 | 112 ± 20^{§} |
| [IU/L] | | | |
| Total protein | 4.9 ± 0.1 | 5.2 ± 0.1 | 5.1 ± 0.1 |
| [g/dl] | | | |
| Alanine transaminase | 408 ± 74 | 310 ± 72 | 233 ± 56 |
| [IU/L] | | | |
| Creatinine | 0.5 ± 0.1 | 0.6 ± 0.0 | 0.5 ± 0.1 |
| [mg/dl] | | | |
| | n = 9 | n = 9 | n = 9 |

*p ≦ 0.05 compound 13 vs. vehicle.
^{§}p ≦ 0.05 compound 13 vs. ALK3-Fc.
One-way ANOVA, adjusted for multiple comparisons using the Bonferroni correction.

In addition to atherosclerosis and vascular calcification, LDLR^{-/-} mice fed a HFD are observed to develop hepatic steatosis. (Hartvigsen, K., et al. A diet-induced hypercholesterolemic murine model to study atherogenesis without obesity and metabolic syndrome. Arterioscler Thromb Vasc Biol 27, 878-885 (2007).) Recent reports have shown that reduction of serum lipoprotein levels could prevent steatosis in LDLR^{-/-} mice. (Wouters, K., et al. Dietary cholesterol, rather than liver steatosis, leads to hepatic inflammation in hyperlipidemic mouse models of nonalcoholic steatohepatitis. Hepatology 48, 474-486 (2008).) Hence, whether treatment with compound 13 could prevent steatosis in LDLR^{-/-} mice was tested. It was observed that LDLR^{-/-} mice fed a HFD for 20 weeks had marked steatosis that could be prevented by treatment with compound 13 (FIG. 9d). Consistent with a reduction in steatosis and associated inflammation, it was observed that treatment with compound 13 reduced serum alkaline phosphatase (ALP) and alanine transaminase (ALT) levels in LDLR^{-/-} mice (Table 1 and 3). These results suggest that inhibition of BMP signaling can prevent hepatic steatosis in LDLR^{-/-} mice likely by reducing lipoprotein levels; however, the possibility of a lipoprotein-independent effect of BMP signaling on hepatic fat accumulation cannot be excluded.

Chemicals and Reagents.

Compound 13 (4-[6-(4-piperazin-1-ylphenyl)pyrazolo[1,5-a]pyrimidin-3-yl]quinoline), was synthesized as previously described. (Cuny, G. D., et al. Structure-activity relationship study of bone morphogenetic protein (BMP) signaling inhibitors. Bioorg Med Chem Lett 18, 4388-4392 (2008).) ALK3-Fc was provided by Acceleron Pharma Inc. (Cambridge, Mass.). OsteoSense 680 and ProSense 750 were obtained from PerkinElmer (Waltham, Mass.). Recombinant human BMP2 was from R&D Systems (Minneapolis, Minn.). Human Oxidized LDL (oxLDL) was from Intracell Corp. (Frederick, Md.). CM-H2DCFDA (Chloromethyl 2′,7′-dichlorodihydrofluorescein diacetate) was from Invitrogen (Eugene, Oreg.). Lucigenin was purchased from Sigma (St. Louis, Mo.).

Animals.

Female mice deficient for the low density lipoprotein receptor (LDLR^{-/-}) on a C57BL/6 background with appropriate control mice (eight weeks of age) were obtained from Jackson Laboratories (Bar Harbor, Me.). Animals were fed a western style diet formulated to match Paigen's Atherogenic Rodent Diet (42% fat, 0.15% cholesterol, 19.5% casein; Research Diets Inc., New Brunswick, N.J.).

Near Infrared Imaging and Estimation of Atherosclerotic Burden.

Fluorescence reflectance imaging (FRI), was performed as previously described. (Aikawa, E., et al. Osteogenesis associates with inflammation in early-stage atherosclerosis evaluated by molecular imaging in vivo. Circulation 116, 2841-2850 (2007); and Aikawa, E., et al. Multimodality molecular imaging identifies proteolytic and osteogenic activities in early aortic valve disease. Circulation 115, 377-386 (2007).) Animals received 150 pi of OsteoSense 680 and 150 μl of ProSense 750 via tail vein injection 24 hours before euthanasia. After dissection, aortae were subjected to ex vivo near infrared fluorescence reflectance imaging using an Odyssey Imaging System (LI-COR Biotechnology, Lincoln, Nebr.), with integrated signal intensities determined for regions of interest using software version 3.0.16.

Quantitative RT-PCR.

Total cellular RNA from either snap frozen tissues or cultured cells was extracted by the Phenol/guanidine method as described. (Chomczynski, P. & Sacchi, N. Single-step method of RNA isolation by acid guanidinium thiocyanate-phenol-chloroform extraction. Anal Biochem 162, 156-159 (1987).) cDNA was synthesized using Moloney murine leukemia virus reverse transcriptase (Promega, Madison, Wis., USA). Quantitative PCR was performed using primer sequences provided in Table 4.

TABLE 4
List of primer sets used
for quantitative RT-PCR.

| | NCBI | |
|---|---|---|
| | Gene ID | Sequence |
| BMP2 forward | 650 | ACCCGCTGTCTTCTAGCGT |
| BMP2 reverse | | CTCAGGACCTCGTCAGAGGG |
| BMP4 forward | 652 | TTCCTGGTAACCGAATGCTGA |
| BMP4 reverse | | CCCTGAATCTCGGCGACTTTT |
| BMP6 forward | 654 | AGCGACACCACAAAGAGTTCA |
| BMP6 reverse | | GCTGATGCTCCTGTAAGACTTGA |
| BMP7 forward | 655 | CGCCGCCTACTACTGTGAG |
| BMP7 reverse | | AGGTGACCACACCCCAAGAT |
| BMP9 forward | 2658 | AGAACGTGAAGGTGGATTTCC |
| BMP9 reverse | | CGCACAATGTTGGACGCTG |

Measurement of Reactive Oxygen Species and NADPH Oxidase Activity.

Cells plated in a 96-well format were pre-treated with compound 13, ALK3-Fc, or vehicle for 30 min followed by treatment with oxidized LDL, BMP2, or vehicle for 20 hours after starvation for six hours. H₂O₂ production with chloromethyl 2′,7′-dichlorodihydrofluorescein diacetate (DCF, 2 μM) and NADPH oxidase activity with lucigenin were measured as previously described. (Ichinose, F., et al. Cardiomyocyte-specific overexpression of nitric oxide synthase 3 prevents myocardial dysfunction in murine models of septic shock. Circ Res 100, 130-139 (2007); Sorescu, G. P., et al. Bone morphogenic protein 4 produced in endothelial cells by oscillatory shear stress induces monocyte adhesion by stimulating reactive oxygen species production from a nox1-based NADPH oxidase. Circ Res 95, 773-779 (2004); and Abid, M. R., Spokes, K. C., Shih, S. C. & Aird, W. C. NADPH oxidase activity selectively modulates vascular endothelial growth factor signaling pathways. J Biol Chem 282, 35373-35385 (2007).)

Histology and Immunohistochemistry.

Aortas were either immediately embedded and cryopreserved using optimal cutting-temperature medium (Sakura Tissue-Tek, Zoeterwoude, The Netherlands) or fixed in paraformaldehyde and embedded in parafin. The presence of calcification was determined by staining tissue sections with Alizarin red or Von Kossa. Lipid accumulation in tissue sections was visualized by staining with Oil Red O. Liver sections were prepared from paraformaldehyde-fixed tissue and stained with hematoxylin and eosin.

For immunofluorescence, frozen sections were post-fixed in cold methanol and incubated with polyclonal antibodies specific for p-SMAD1/5/8 (1:100, Cell Signaling, Danvers, Mass.) or MAC2 (1:100, Cedarlane, Burlington, ON) followed by FITC labeled Goat Anti-Rabbit IgG or Rhodamine Goat Anti-Rat IgG (both Jackson Immuno Research, West Grove, Pa.), respectively.

Serum Analysis.

Triglycerides, total cholesterol, white blood count, hematocrit, hemoglobin, and platelets were analyzed using a HemaTrue™ Hematology Analyzer (Heska AG, Switzerland). Urea nitrogen, glucose, alkaline phosphatase, total protein, alanine transaminase and creatinine were determined using a Spotchem EZ SP-4430 POCT analyzer (Arkray, Inc., Kyoto, Japan). HDL and LDL levels were determined using a fluorescence quantification kit (K613-100, Biovision, Mountain View, Calif.)

HMG-CoA Reductase Activity.

Enzyme activity was quantified using a commercially available Assay Kit (CS 1090 HMG-CoA Reductase Kit, Sigma-Aldrich, St. Louis, Mo.)

Statistical Analysis.

Statistical analysis was performed using SPSS 14.0 Data package for Windows (SPSS, Chicago, Ill.) and Graph Pad Prism 5.02 (GraphPad Software, La Jolla, Calif.). Data are reported as mean±SEM. Samples were compared, for example, by Student's t-test. A p≦0.05 was regarded as statistically significant.

Tissue Culture.

Hepatoma G2 (HepG2) cells and human aortic endothelial cells (HAECs) were purchased from the American type culture collection (Manassas, Va.). HepG2 cells were maintained in Eagle's Minimum Essential Medium (EMEM) supplemented with 10% fetal bovine serum, 100 units/ml of penicillin, 0.1 mg/ml of streptomycin and glutamine. HAECs were cultured in endothelial cell basal medium (EBM 2) supplemented with the EBM2 bullet kit (Lonza, Basel, Switzerland). For experiments, cells were seeded into 6 or 12-well plates (BD Falcon, Franklin Lakes, N.J.) at a concentration of 0.5×10⁶ cells per 5 mL of media. HAECs were maintained in EBM 2 with 0.1% FBS without additional growth factors for all experiments. HepG2 cells were grown to 70% confluence before they were incubated in EMEM enriched with 0.1% FBS. ApoB-100 measurements were performed in supernatants from HepG2 that had been incubated in EMEM containing 0.5% bovine serum albumin using a commercially available Human ApoB-100 ELISA kit (Mabtech AB, Nacka Strand, Sweden). BMP2 protein measurements were performed in supernatants from HAECs that had been incubated in EBM2 containing 0.1% FBS using a BMP2 ELISA kit (R&D Systems).

### Example 2

### Establishment of a Mouse Model of Atheromatous and Vascular Calcific Disease

The inventors' objectives are to demonstrate the effect of pharmacologic BMP inhibition upon the development of (i) atheromatous disease burden, and (ii) vascular calcification in an accepted animal model of atherosclerosis, in order to provide potential proof-of-concept that BMP inhibition can be an effective strategy for preventing atherosclerosis or limiting its progression.

BMPs are multifunctional protein ligands which form a subset of the transforming growth factor-β (TGF-β) family of signaling proteins (Feng, X. H. & Derynck, R., Annu Rev Cell Dev Biol 21, 659-693 (2005)). BMPs, originally identified by their ability to induce ectopic bone formation, serve broad roles in gastrulation, developmental patterning, and organ formation. In the adult organism, BMP signals serve principally to mediate injury repair and inflammation. Aberrant BMP signaling may contribute to a number of acquired diseases, perhaps via inappropriate activation of repair or inflammatory responses. Specifically, it has been proposed that BMP signals contribute to atherosclerosis, since BMPs and many of the BMP-induced gene products which affect matrix remodeling are overexpressed in early atherosclerotic lesions, and may promote plaque formation and progression (Bostrom, K. & Demer, L. L., Crit Rev Eukaryot Gene Expr 10, 151-158 (2000); Bostrom, K., et al., J Clin Invest 91, 1800-1809 (1993); Tintut, Y., et al., Circulation 108, 2505-2510 (2003)). Over time, BMP signals may also induce resident or circulating progenitors to form the cells of bone, including osteoblasts and chondroblasts, and cause calcification of vessels (Tintut, Y., et al., 2003, supra). In addition to increasing risk of cardiovascular events and mortality, severe calcific vascular disease is particularly problematic in that it can interfere with the body's ability to restore adequate circulation to the coronary vessels by angioplasty or bypass surgery. In these studies, the inventors investigated whether atherosclerotic and calcific lesions can be ameliorated or prevented, if signals which contribute to their progression can be intercepted during their formation. The proof-of-principle experiments described in this report tested the effects of a novel pharmacologic inhibitor of BMP signaling in an accepted animal model of atheromatous disease.

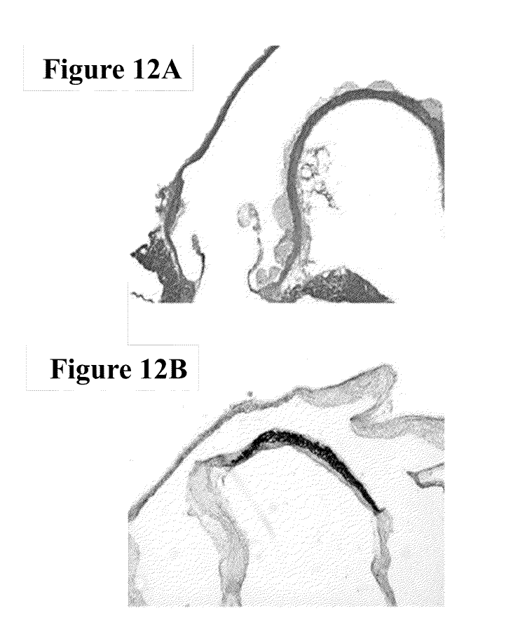

The inventors observed in LDLr−/− mice which were started on a high fat diet at 8 weeks of life, that within 16-20 weeks, profound atheromatous and vascular calcific lesions developed throughout the arterial tree, including the aorta and its major branch vessels (FIG. 12). When fed a high fat diet, low density lipoprotein receptor-deficient (LDLr−/−) mice are genetically predisposed to high cholesterol levels, and consequently the development of atherosclerotic and calcific vascular lesions, occurring in a manner of weeks only after challenge with a high cholesterol and high lipd diet (Aikawa, E., et al., Circulation 116, 2841-2850 (2007); Aikawa, E., et al., Circulation 115, 377-386 (2007); Ohshima, S., et al., J Nucl Med 50, 612-617 (2009); Isobe, S., et al., J Nucl Med 47, 1497-1505 (2006)). In order to quantify and assess the degree of atheromatous and vascular calcification disease, the inventors employed traditional immunochemical techniques (Oil Red O staining for lipid deposition, and Von Kossa mineral staining for evidence of calcification) on explanted vessel tissue samples. In addition, the inventors employed several novel molecular imaging probes which have been validated to detect the presence of osteogenic or bone-forming activity (Osteosense, a bisphosphonate probe which binds to vessel-associated osteoblasts), and vascular inflammation associated with atheroma (Prosense, a cathepsin substrate which binds vessel-associated macrophages), the intensity of either of which can be quantitated by near-infrared fluorescence reflectance imaging as previously described (Aikawa, E., et al., (2007) and (2006) supra).

As has been described previously, these mice had gross evidence of intimal lesions in the minor curvature of the aorta (FIG. 12A), and developed in addition calcification of the vessel media as detected by Von Kossa mineral staining (FIG. 12B). These findings were found with 100% penetrance in LDLr−/− mice given a high fat diet, and were not found in control mutant mice given a normal diet, or in wild-type (C57BL/6) control mice given a high fat diet. This protocol yielded a robust model of atherosclerosis and atherosclerosis-associated vascular calcification in the context of hypercholesterolemia and an atherogenic diet.

### Example 3

### A BMP Inhibitor can Inhibit the Development of Vascular Calcification and Macrophage-Mediated Inflammation Associated with Atheromatous Disease

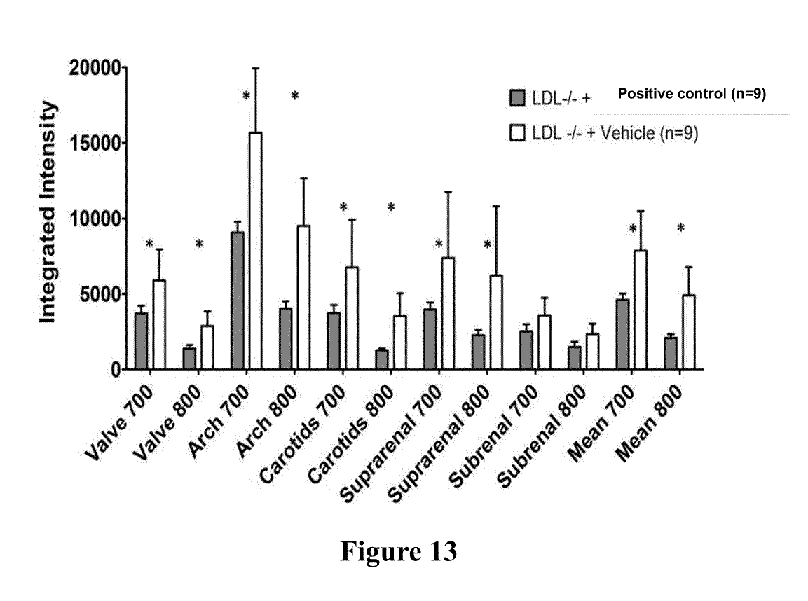

LDLr−/− mice were treated with a BMP inhibitor positive control compound (compound 13, 2.5 mg/kg/d intraperitoneally) or vehicle (saline) for 20 weeks following the initiation of a high fat diet. Mice were injected with Osteosense (to label sites of bone-forming activity via osteoblast binding of this probe) and Prosense (to label sites of macrophage-mediated inflammation). Aortae were explanted and subjected to fluorescence reflectance imaging (LICOR Odyssey imager). Fluorescence in the 700 nm channel (Osteosense) revealed diminished fluorescence in the aortae of the BMP inhibitor positive control compound-treated as compared to vehicle-treated mice (data not shown). Significant differences in macrophage and osteoblast staining were observed throughout the vascular tree in a cohort of treated and control mice (n=10 each). In examining a cohort of 10 vehicle-treated and 10 drug-treated mice, quantitation of the Osteosense signal revealed significant attenuation of osteoblast activity throughout the arterial tree (data not shown), particularly at key areas which are known to be sites of intense atherosclerotic remodeling, including the aortic valve and root, the aortic arch, the carotid bifurcations, and the suprarenal bifurcations. The BMP inhibitor positive control compound-treated aortae had severely diminished evidence of osteogenesis on the basis of the osteoblast probe intensity at 700 nm. Examination of fluorescence in the 800 nm channel (Prosense) revealed diminished macrophage activity in the vessels of the BMP inhibitor positive control compound-treated versus vehicle-treated mice (data not shown). This indicates that the BMP inhibitor positive control compound-treated aortae had severely diminished evidence of macrophage activity on the basis of macrophage probe intensity at 800 nm. The diminished macrophage activity was significantly decreased with drug treatment, when quantitated at the aortic root, arch, and carotid bifurcations (FIG. 13). These results demonstrate that small molecule pharmacologic inhibition of the BMP signaling pathway with the BMP inhibitor positive control compound lead to diminished osteogenic activity (required for vascular calcification) and decreased vascular inflammation, both of which have been shown to vary in proportion to the total atherosclerotic burden (Aikawa, E., et al., Circulation 116, 2841-2850 (2007)). These results suggested that BMP signaling regulates the process of atherogenesis.

To confirm that BMP signaling has a direct impact on atherogenesis, the aortae explanted from the BMP inhibitor positive control compound-treated and vehicle-treated LDLr−/− mice after 20 weeks were subjected to Oil Red O staining to mark lipid-rich plaques. Aortae were fixed and labeled with lipid-specific stain Oil Red O. The total atheroma burden was observed to be consistently greater in vehicle-treated mice as compared to the BMP inhibitor positive control compound-treated mice by this technique (n=3 each, representative data shown). The size and extent of Oil Red O-stained atheromatous lesions were found to be consistently more severe in vehicle-treated than the BMP inhibitor positive control compound-treated mice (data not shown), supporting the interpretation that diminished osteoblast and macrophage activity (based on Osteosense and Prosense data) reflected diminished plaque formation. These data corroborate the interpretation that BMP inhibition diminishes the formation of atheroma itself.

### Example 4

### Verification that the BMP Inhibitor Positive Control Compound Inhibits BMP Signaling Activity (Activated SMAD1/5/8) Associated with Atheromatous Lesion Formation

LDLr−/− mice were started on a hypercholesterolemic diet at 8 weeks, and treated with either vehicle (saline) or a BMP inhibitor positive control compound (compound 13, 2.5 mg/kg/d intraperitoneally) for an additional 8 weeks. Aortae were harvested and fixed, and then stained with antibodies sensitive for the BMP effector molecule, phosphorylated-SMAD 1/5/8, and counterstained with DAPI nuclear stain. Within 6-8 weeks of being subjected to a high fat diet, LDR−/− mice developed fatty lesions in the intima of the aortic root, based on traditional histochemical staining techniques (data not shown). The BMP inhibitor positive control compound-treated animals had reduced intimal atheroma formation as compared to vehicle-treated animals. Atheroma formation was associated with prominent staining of phosphorylated-SMAD1/5/8 in vehicle-treated animals, which was greatly diminished in the BMP inhibitor positive control compound-treated animals. When subjected to immunofluorescent staining for the phosphorylated form of SMAD1/5/8, an effector molecule which is recruited by the BMP signaling pathway, the aortae of vehicle-treated mice revealed intense nuclear staining in a manner typical of nuclear-localized activated SMAD1/5/8 (data not shown) (Feng, X. H. & Derynck, R., Annu Rev Cell Dev Biol 21, 659-693 (2005)). Thus, the cellular components of lipid rich plaques, predominantly macrophage-derived foam cells, had evidence of intense activation of the BMP signaling pathway. In contrast, the lipid plaques found in the BMP inhibitor positive control compound-treated mice, which were diminished in size and extent as compared to those in vehicle-treated mice, had also diminished intensity of staining for the phosphorylated form of SMAD1/5/8 (data not shown). Thus, hypercholesterolemic mice had evidence of intense BIVIP signaling pathway activation in the cellular components of atheromatous lesions, and treatment of hypercholesterolemic mice with the BMP inhibitor positive control compound diminished the activation of the BMP signaling pathway in these lesions.

### Example 5

### Demonstration that a Soluble Recombinant BMP Receptor Ectodomain Inhibits BMP Signaling Activity (Activated SMAD1/5/8) Associated with Atheromatous Lesion Formation and Also Inhibits Macrophage-Mediated Inflammation

Inflammatory activity, a surrogate of atherosclerotic plaque burden, was assessed by near-IR fluorescence of Prosense (fluor-cathepsin substrate) at 700 nM. Ten individual mice were used in each treatment group. Prosense uptake was significantly reduced by treatment with ALK3-Fc (2 mg/kg IP QOD) or a BIVIP inhibitor positive control compound (compound 13, 2.5 mg IP QD) as compared to vehicle for 6 weeks following the initiation of an atherogenic (Paigen) diet in adult (8 wk) LDLR−/− C57BL/6 mice, particularly in the aortic root and aortic arch (data not shown). This result indicates that macrophage-mediated inflammation is qualitatively decreased in the central arterial vascular bed of atherogenic animals by recombinant or small-molecule BMP inhibitors.

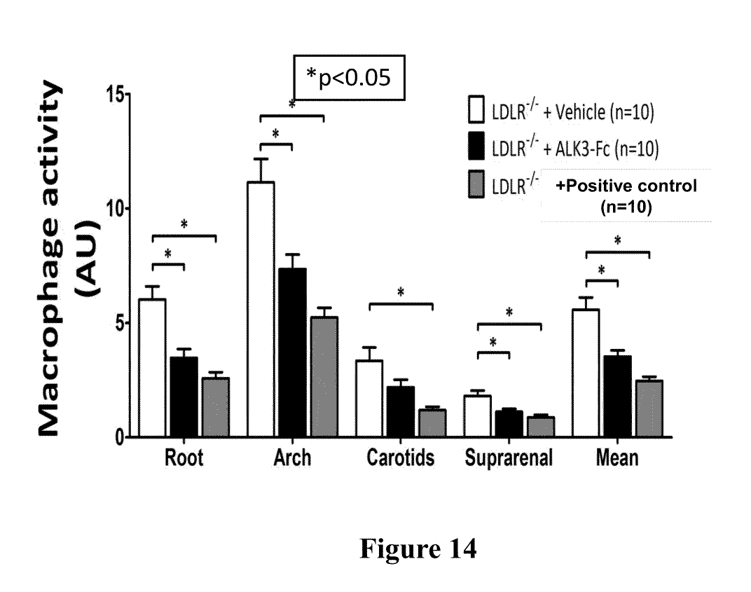

Inflammatory activity, a surrogate of atherosclerotic plaque burden, was assessed by integrated intensity of near-IR fluorescence of Prosense (fluor-cathepsin substrate) at 700 nM. Prosense integrated intensity was significantly inhibited by treatment with ALK3-Fc (2 mg/kg IP QOD) or BMP inhibitor positive control compound (compound 13, 2.5 mg IP QD) versus vehicle for 6 weeks following the initiation of an atherogenic (Paigen) diet in adult (8 wk) LDLR−/− C57BL/6 mice, particularly in the aortic valve, root, arch, and suprarenal areas of the aorta. FIG. 14 shows that macrophage-mediated inflammation is quantitatively decreased in the central arterial vascular bed of atherogenic animals by recombinant or small-molecule BMP inhibitors. Each bar represents the mean±SEM of measurements obtained on tissues obtained from 10 individual mice per group with significant differences versus vehicle-treated animals indicated.

LDLR−/− deficient mice were initiated on an atherogenic diet (Paigen) at 8 weeks of age, and administered either vehicle, a BMP inhibitor positive control compound (compound 13, 2.5 mg/kg IP daily) or ALK3-Fc (2 mg/kg IP every other day). Each treatment group consisted of a total of 10 mice. After 4 weeks of atherogenic diet and drug or vehicle treatment, the animals were sacrificed. The frontal plane sections of the aortic arch were dissected out and stained for macrophage marker (MAC2) and counterstained with DAPI. The BMP inhibitor positive control compound-treated mice exhibited decreased lesion formation overall, and decreased staining for MAC2. ALK3-Fc-treated mice also exhibited profoundly decreased lesion formation and MAC2 staining. This result indicates that BMP inhibitors can effectively limit the development of early atheromatous lesions in atherogenic mice.

LDLR−/− deficient mice were initiated on an atherogenic diet (Paigen) at 8 weeks of age, and administered either vehicle, a BMP inhibitor positive control compound (compound 13, 2.5 mg/kg IP daily) or ALK3-Fc (0.2 mg/kg IP every other day). After 6 weeks of atherogenic diet and treatment, the animals were sacrificed. The frontal plane sections of the aortic arch were dissected out and stained for phosphorylated SMAD1/5/8 and counterstained with DAPI. Vehicle-treated mice exhibited early atheromatous lesion formation associated with the activation of SMAD1/5/8 in endothelial, smooth muscle, as well as MAC2+ foam cell populations (data not shown). Control sections stained with only secondary Ab exhibited weak background fluorescence in the internal elastic lamina (data not shown). The BMP inhibitor positive control compound-treated mice exhibited decreased lesion formation overall, and decreased staining for phosphorylated SMAD1/5/8 (data not shown). ALK3-Fc-treated mice also exhibited profoundly decreased lesion formation and phosphorylated SMAD1/5/8 staining (data not shown). This result indicates that BMP inhibitor treatment effectively inhibits activation of BMP-SMAD signaling in the vasculature of atherogenic mice.

### Example 6

### Bone Morphogenic Protein Signaling is Required for Vascular Calcification in a Murine Model of Matrix GLA Protein Deficiency

Matrix GLA protein (MGP) is a mineral-binding extracellular matrix protein that is thought to prevent vessel calcification by sequestration of calcium ions. However, MGP also inhibits bone morphogenetic protein (BMP) signaling. MGP−/− mice exhibit severe medial arterial calcification by 2 wks and die by 6 wks from aortic aneurysm and rupture. The inventors tested whether MGP prevented vascular calcification via its effects on BMP signaling.

MGP−/− mice were treated with either vehicle or the small molecule BMP type I receptor inhibitor compound 13 (2.5 mg/kg once daily IP) from day 1 to 28. Compound 13 is used as a BMP inhibitor positive control compound in these experiments. Whole aortas were harvested for phospho-Smad 1/5/8 (P-Smad) immunohistochemistry, a marker of BMP signaling, and for Alizarin Red staining of calcium. Osteogenic activity in aortas was visualized ex vivo by the uptake of a fluorescent bisphosphonate imaging probe. MGP−/− mice were also treated with vehicle or compound 13 to ascertain if inhibition of BMP signaling could impact survival, using Kaplan-Meier and Cox regression analysis.

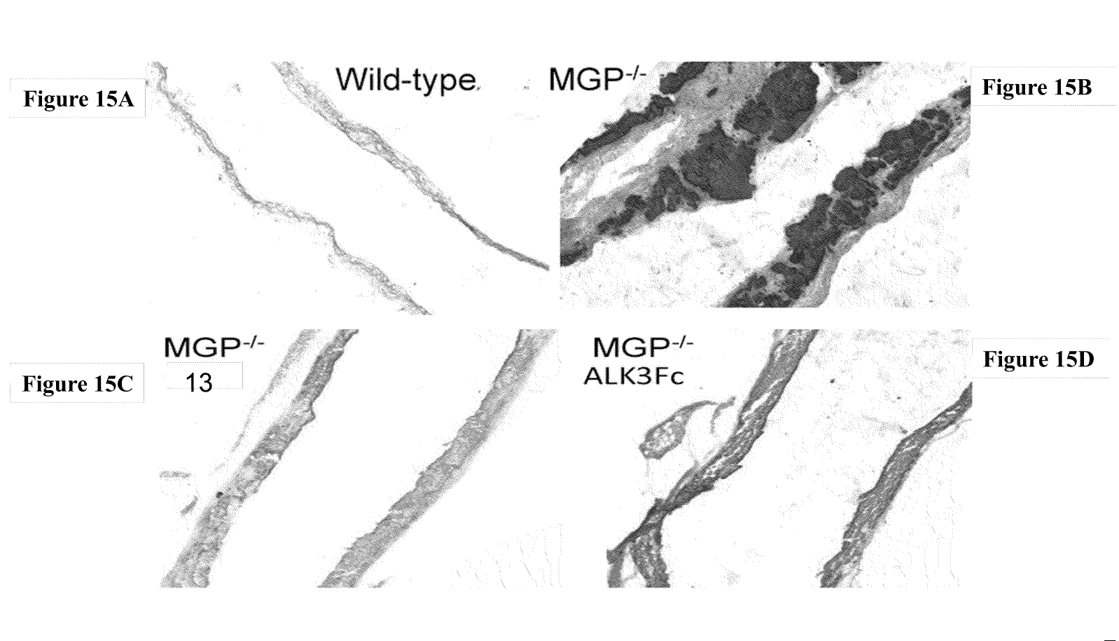

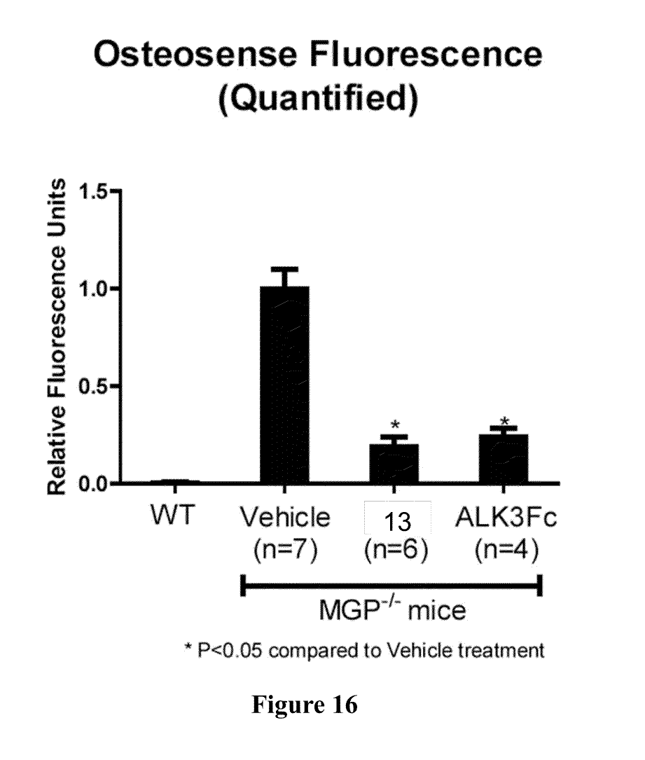

MGP−/− aortas demonstrated increased P-Smad compared to wild-type mice (data not shows). Compound 13 treatment of MGP−/− mice reduced aortic P-Smad levels and was associated with a reduction in tissue calcium levels (FIG. 15C). Similar, ALK3-Fc treatment of MGP−/− mice also exhibited significantly reduced tissue calcification in the aorta (FIG. 15D). Pharmacologic inhibition of BMP signaling in MGP−/− mice resulted in an 81% reduction in aortic osteogenic activity compared to vehicle-treated controls (n=6 in each group; normalized average intensity±SEM, 0.19±0.05 vs 1.0±0.10, P<0.0001) (FIG. 16), with similar reductions observed at the aortic arch and the abdominal aorta. Compound 13 treated mice exhibited improved survival compared to vehicle-treated controls (n=10 in each group; Cox hazard ratio 0.04, 95% CI 0.01-0.17, P<0.001).

Accordingly, these data support the conclusion that MGP prevents vascular calcification primarily via its impact on BMP signaling. Pharmacologic BMP inhibition improves survival in MGP−/− mice and may represent an important therapeutic target in the treatment of human vascular disease.

### Example 7

### Enhanced BMP Signaling as the Primary Mechanism by which MGP Deficiency Induces Vascular Calcification

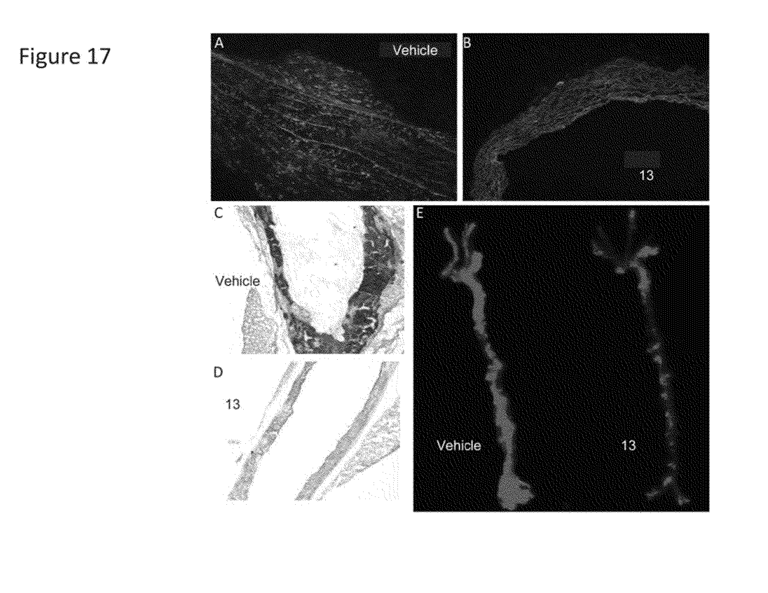

MGP−/− mice were treated with intraperitoneal (IP) injections of either vehicle or compound 13 at 2.5 mg/kg/day from day 1-28. At day 28, aortas were harvested for both histology and RNA isolation. Immunohistochemistry (IHC) for Smad 1/5/8 phosphorylation confirmed that compound 13 treatment reduced BMP signaling (FIG. 17A-B). Alizarin red staining for tissue calcium (FIG. 17C-D) revealed that compound 13 reduced vessel calcification. To further quantify aortic calcium, a fluorescent bisphosphonate agent that specifically binds to hydroxyapatite (OsteoSense 680 nm, Perkin Elmer) was used. (Aikawa E, et al. Multimodality molecular imaging identifies proteolytic and osteogenic activities in early aortic valve disease. Circulation. 2007; 115(3):377-386 and Zaheer A, et al. In vivo near-infrared fluorescence imaging of osteoblastic activity. Nature biotechnology. 2001; 19(12):1148-1154.) MGP−/− mice at 27 days of age treated with vehicle or compound 13 received OsteoSense via tail vein, and 24 h later, aortas were harvested for imaging. MGP−/− mice treated with compound 13 exhibited a marked reduction in arterial calcification (FIG. 17E). The reduction in BMP signaling and aortic calcification observed with compound 13 treatment of MGP−/− mice was associated with a >5-fold reduction in aortic Wnt3a gene expression, implicating the Wnt signaling pathway as an important mediator of BMP-dependent osteogenic differentiation (P<0.01).

MGP−/− mice develop histologically-evident aortic calcification (FIG. 17C) by 2 weeks of age associated with immunohistologic evidence of Smad 1/5/8 phosphorylation (FIG. 17A), whereas wild-type mice exhibit no arterial calcification and have markedly lower levels of Smad 1/5/8 phosphorylation (data not shown). Wnt3a gene expression was two-fold greater in aortas from 4-wk old MGP−/− mice than in those of aged-matched controls (P=0.02). Wnt3a expression levels correlated with the degree of vessel wall calcification. Vascular calcification progresses with age, and all MGP−/− mice die 6-8 weeks after birth. (Luo, G. et al., Spontaneous calcification of arteries and cartilage in mice lacking matrix GLA protein. Nature. 1997; 386(6620):78-81.)

### Example 8

### Pharmacologic Inhibition of BMP Signaling Reduces Aortic Calcification in MGP Deficiency

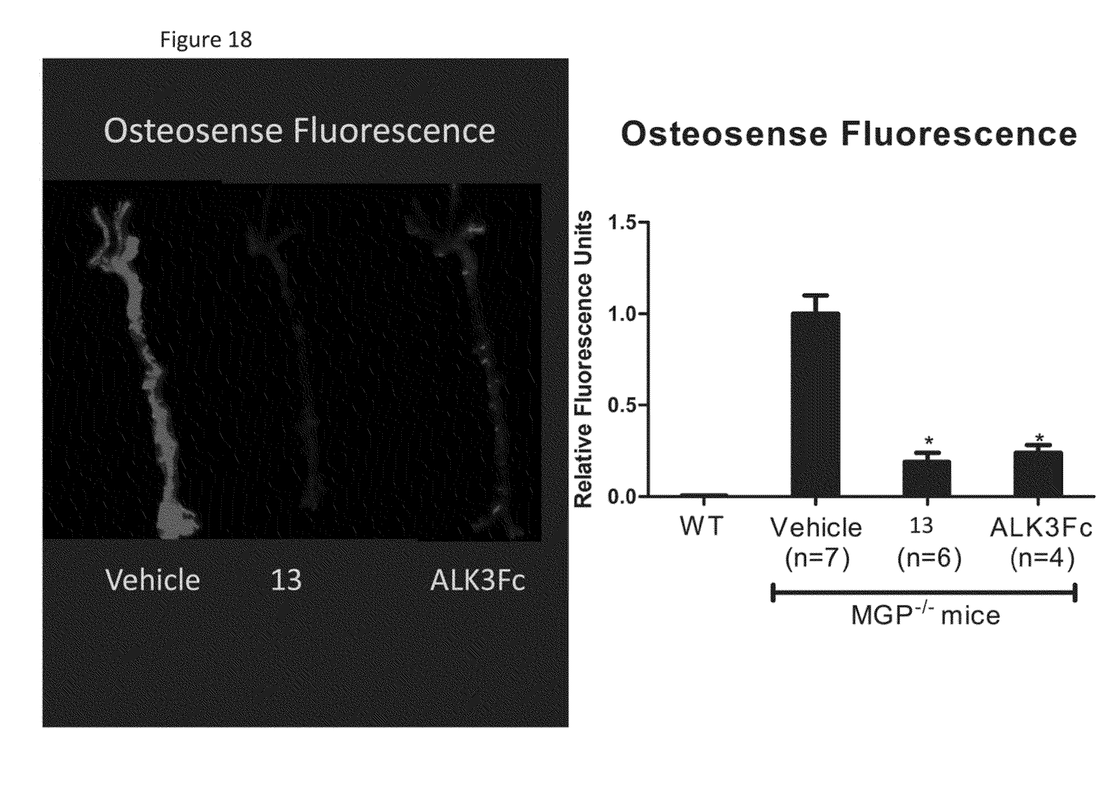

MGP−/− mice were treated with vehicle (n=7), compound 13 (2.5 mg/kg/day IP, n=6), or ALK3-Fc (2 mg/kg QOD IP, n=4) beginning on day 1 after birth. At day 27, mice were injected via the tail vein with a fluorescent bisphosphonate probe (Osteosense 680 nm, 2 nmol per mouse) that targets tissue calcification. Twenty-four hours later, aortae were harvested and imaged. Results are represented in FIG. 18, wherein the left-hand panel shows representative aortae from a vehicle-treated mouse, a mouse treated with compound 13 and a mouse treated with Alk3-Fc. These results demonstrate that treatment with either compound 13 or Alk3-Fc reduces vascular calcification. The right-hand panel quantifies the fluorescent intensity from the aortae, demonstrating an 80% reduction in vascular calcification with pharmacologic BMP inhibition, wherein * indicates P<0.05 compared to Vehicle treatment.

### Example 9

### Pharmacologic Inhibition of BMP Signaling Improves Survival in MGP Deficiency

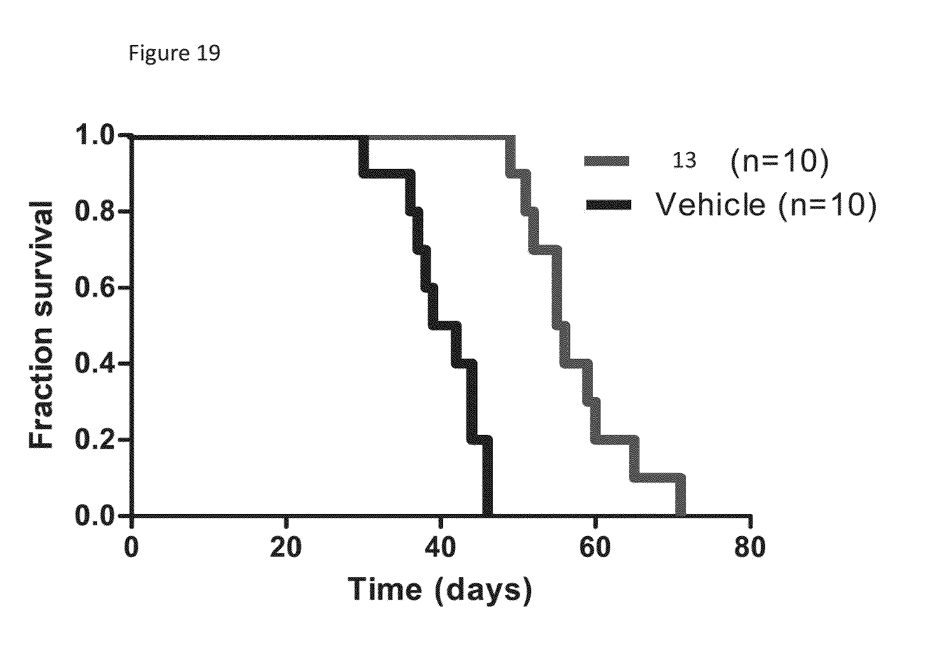

Twenty MGP−/− mice were treated either with vehicle (n=10) or compound 13 (2.5 mg/kg/day IP, n=10) beginning at day 1 of life and followed for survival. Kaplan-Meier survival curves are presented in FIG. 19. Treatment with compound 13 improved survival (Log Rank P=0.002) with a Cox hazard ratio of 0.04.

All publications and patents cited herein are hereby incorporated by reference in their entirety.

Those skilled in the art will recognize, or be able to ascertain using no more than routine experimentation, many equivalents to the specific embodiments of the invention described herein. Such equivalents are intended to be encompassed by the following claims.

1-19. (canceled)

20. A method of reducing circulating levels of ApoB-100, LDL, total cholesterol, or any combination thereof in a human subject suffering from hypercholesterolemia, hyperlipidemia, hyperlipoproteinemia, hepatic steatosis, non-alcoholic fatty liver disease (NAFLD), steatosis-induced liver injury, fibrosis, cirrhosis, or non-alcoholic steatohepatitis (NASH), thereby reducing risk of primary or secondary cardiovascular events, comprising administering an effective amount of a compound having a structure of Formula I: 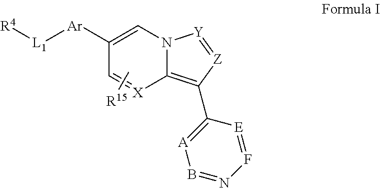
wherein
X and Y are independently selected from CR¹⁵ and N;
Z is selected from CR³ and N;
Ar is selected from substituted or unsubstituted aryl and heteroaryl;
L₁ is absent or selected from substituted or unsubstituted alkyl and heteroalkyl;
A and B, independently for each occurrence, are selected from CR¹⁶ and N;
E and F, independently for each occurrence, are selected from CR⁵ and N;
no more than two of A, B, E, and F are N; and
either E and F are both CR⁵ and both occurrences of R⁵ taken together with E and F form a ring, or L₁ is absent;
R³ is selected from H and substituted or unsubstituted alkyl, cycloalkyl, halogen, acylamino, carbamate, cyano, sulfonyl, sulfoxido, sulfamoyl, or sulfonamido;
R⁴ is selected from H and substituted or unsubstituted alkenyl, alkynyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido;
R⁵, independently for each occurrence, is selected from H and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido, or two occurrences of R⁵ taken together with the atoms to which they are attached form a substituted or unsubstituted 5- or 6-membered cycloalkyl, heterocycloalkyl, aryl, or heteroaryl ring;
R¹⁵, independently for each occurrence, is selected from H and substituted or unsubstituted alkyl, cycloalkyl, heterocyclyl, cycloalkylalkyl, heterocyclylalkyl, halogen, acylamino, carbamate, cyano, sulfonyl, sulfoxido, sulfamoyl, or sulfonamido;
R¹⁶, independently for each occurrence, is absent or is selected from H and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido,
or a pharmaceutically acceptable salt, ester, or prodrug thereof.

21. The method of claim 20, wherein A and B are each CH.

22. The method of claim 20, wherein E and F are each CR⁵, and the atoms to which both instances of R⁵ are attached form a 6-membered ring.

23. The method of claim 22, wherein E and F together represent the group 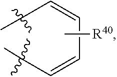
wherein R⁴⁰ is absent or represents from 1-4 substituents selected from substituted or unsubstituted alkyl, cycloalkyl, halogen, acylamino, carbamate, cyano, sulfonyl, sulfoxido, sulfamoyl, or sulfonamido.

24. The method of claim 20, wherein L₁ has a structure 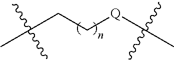
wherein
Q is selected from CR¹⁰R¹¹, NR¹², O, S, S(O), and SO₂; and
R¹⁰ and R¹¹, independently for each occurrence, are selected from H and substituted or unsubstituted alkyl, cycloalkyl, heterocyclyl, cycloalkylalkyl, heterocyclylalkyl, amino, acylamino, carbamate, amido, amidino, cyano, sulfonyl, sulfoxido, sulfamoyl, or sulfonamido;
R¹² selected from H and substituted or unsubstituted alkyl, cycloalkyl, heterocyclyl, heterocyclylalkyl, amino, acylamino, carbamate, amido, amidino, sulfonyl, sulfamoyl, or sulfonamido and
n is an integer from 0-4.

25. The method of claim 20, wherein R⁴ is selected from 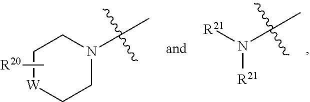
wherein
W is absent or is C(R²¹)₂, O, or NR²¹;
R²⁰ is absent or is selected from substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfoxido, sulfamoyl, and sulfonamido; and
R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamido.

26. The method of claim 20, wherein Ar is a 6-membered aryl or heteroaryl ring.

27. The method of claim 26, wherein L₁ is disposed on the para-position of Ar relative to the bicyclic core.

28. A method of treating a disease, disorders, or syndromes associated with defects in lipid absorption or metabolism or caused by hyperlipidemia in a subject; reducing primary and secondary cardiovascular events arising from coronary, cerebral, or peripheral vascular disease in a subject; preventing cardiovascular disease in a subject with elevated markers of cardiovascular risk; or preventing or treating hepatic dysfunction in a subject associated with nonalcoholic fatty liver disease (NAFLD), steatosis-induced liver injury, fibrosis, cirrhosis, or non-alcoholic steatohepatitis (NASH), comprising administering to a human subject an effective amount of a compound having a structure of Formula I: 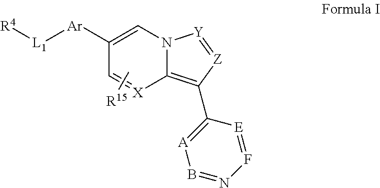
wherein
X and Y are independently selected from CR¹⁵ and N;
Z is selected from CR³ and N;
Ar is selected from substituted or unsubstituted aryl and heteroaryl;
L₁ is absent or selected from substituted or unsubstituted alkyl and heteroalkyl;
A and B, independently for each occurrence, are selected from CR¹⁶ and N;
E and F, independently for each occurrence, are selected from CR⁵ and N;
no more than two of A, B, E, and F are N; and
either E and F are both CR⁵ and both occurrences of R⁵ taken together with E and F form a ring, or L₁ is absent;
R³ is selected from H and substituted or unsubstituted alkyl, cycloalkyl, halogen, acylamino, carbamate, cyano, sulfonyl, sulfoxido, sulfamoyl, or sulfonamido;
R⁴ is selected from H and substituted or unsubstituted alkenyl, alkynyl, cycloalkyl, heterocyclyl, aryl, heteroaryl, acyl, carboxyl, ester, hydroxyl, alkoxyl, alkylthio, acyloxy, amino, acylamino, carbamate, amido, amidino, sulfonyl, sulfoxido, sulfamoyl, or sulfonamido;
R⁵, independently for each occurrence, is selected from H and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido, or two occurrences of R⁵ taken together with the atoms to which they are attached form a substituted or unsubstituted 5- or 6-membered cycloalkyl, heterocycloalkyl, aryl, or heteroaryl ring;
R¹⁵, independently for each occurrence, is selected from H and substituted or unsubstituted alkyl, cycloalkyl, heterocyclyl, cycloalkylalkyl, heterocyclylalkyl, halogen, acylamino, carbamate, cyano, sulfonyl, sulfoxido, sulfamoyl, or sulfonamido;
R¹⁶, independently for each occurrence, is absent or is selected from H and substituted or unsubstituted alkyl, alkenyl, alkynyl, aralkyl, cycloalkyl, heterocyclyl, aryl, heteroaryl, heteroaralkyl, cycloalkylalkyl, heterocyclylalkyl, halogen, acyl, carboxyl, ester, hydroxyl, alkoxyl, alkylthio, acyloxy, amino, acylamino, carbamate, amido, amidino, cyano, sulfonyl, sulfoxido, sulfamoyl, or sulfonamido,
or a pharmaceutically acceptable salt, ester, or prodrug thereof.

29. The method of claim 20, wherein the human subject is suffering from hypercholesterolemia, hyperlipidemia, or hyperlipoproteinemia, and the hypercholesterolemia, hyperlipidemia, or hyperlipoproteinemia is congenital hypercholesterolemia, hyperlipidemia, or hyperlipoproteinemia.

30. The method of claim 20, wherein the human subject is suffering from hypercholesterolemia, hyperlipidemia, or hyperlipoproteinemia, and the hypercholesterolemia, hyperlipidemia, or hyperlipoproteinemia is autosomal dominant hypercholesterolemia (ADH), familial hypercholesterolemia (FH), polygenic hypercholesterolemia, familial combined hyperlipidemia (FCHL), hyperapobetalipoproteinemia, or small dense LDL syndrome (LDL phenotype B).

31. The method of claim 20, wherein the human subject is suffering from hypercholesterolemia, hyperlipidemia, or hyperlipoproteinemia, and the hypercholesterolemia, hyperlipidemia, hyperlipoproteinemia is acquired hypercholesterolemia, hyperlipidemia, or hyperlipoproteinemia.

32. The method of claim 20, wherein the human subject is suffering from hypercholesterolemia, hyperlipidemia, or hyperlipoproteinemia associated with diabetes mellitus, hyperlipidemic diet and/or sedentary lifestyle, obesity, metabolic syndrome, intrinsic or secondary liver disease, biliary cirrhosis or other bile stasis disorders, alcoholism, pancreatitis, nephrotic syndrome, endstage renal disease, hypothyroidism, iatrogenesis due to administration of thiazides, beta-blockers, retinoids, highly active antiretroviral agents, estrogen, progestins, or glucocorticoids.
